(19) United States
(12) Patent Application Publication
(10) Pub. No.: US 2001/0046499 A1
(43) Pub. Date: Nov. 29, 2001

(54) Tick antigens and compositions and methods comprising them

(76) Inventors: Fred S. Kantor, Orange, CT (US); Erol Fikrig, Guilford, CT (US); Subrata Das, New Haven, CT (US)

Correspondence Address: FISH & NEAVE, 1251 AVENUE OF THE AMERICAS, 50TH FLOOR, NEW YORK, NY, 10020-1105, US

(21) Appl. No.: 09/728,914

(22) Filed: Dec. 1, 2000

Related U.S. Application Data
(60) Provisional application No. 60/169,048, filed on Dec. 3, 1999.
(60) Provisional application No. 60/240,716, filed on Oct. 16, 2000.

(51) Int. Cl. A61K 39/00; C12N 9/00; C07H 21/04; C12P 21/02

(52) U.S. Cl. 424/185.1; 435/69.3; 435/325; 536/23.2; 435/183

(57) ABSTRACT

Methods and compositions for conferring tick immunity and preventing or reducing the transmission of tick-borne pathogens. Tick polypeptides, fragments and derivatives; fusion and multimeric proteins comprising the polypeptides, fragments or derivatives; nucleic acid molecules encoding them; antibodies directed against the polypeptides, fusion proteins or multimeric proteins and compositions comprising the antibodies. Vaccines comprising the polypeptides, fragments or derivatives, alone or in addition to other protective polypeptides. Methods comprising the polypeptides, antibodies and vaccines.

This invention was made with government support under Grant numbers A130548-08, ROI-41008-01 awarded by the National Institutes of Health and CCUI 14669-02 awarded by the Center for Disease Control. The government may have certain rights in the invention.

### TECHNICAL FIELD OF THE INVENTION

This invention relates to compositions and methods for conferring immunity to tick bites and for the prevention of tick-borne diseases.

More particularly, this invention relates to tick antigens and the nucleic acid sequences which encode them. Such polypeptides and nucleic acid sequences and kits comprising them are useful to detect tick bites and tick immunity in a subject and in pharmaceutical compositions and vaccines to elicit an immune response which is effective to prevent or lessen the duration of tick attachment or feeding and to prevent or lessen infection of a host with tick-borne pathogens. Also within the scope of this invention is an antibody or an antigen-binding portion thereof that specifically bind a polypeptide of the invention and compositions comprising such an antibody or an antigen-binding portion.

This invention also relates to vaccines or pharmaceutical compositions comprising one or more of the polypeptides or antibodies of this invention. Also within the scope of this invention are diagnostic kits comprising a polypeptide, nucleic acid or antibody of this invention.

This invention also relates to methods for using the aforementioned polypeptides, nucleic acid sequences and antibodies.

### BACKGROUND OF THE INVENTION

Ticks are the most common vector transmitting diseases to humans in the United States [CDC, 1989. Lyme Disease—United States, 1987 and 1988. MMWR Morb. Mortal. Wkly. Rep., 38; 668-672]. Ticks transmit the agents of important human diseases, such as Lyme disease, babesiosis, Rocky Mountain spotted fever, ehrlichiosis, and tick-borne encephalitis. The incidence of tick-borne disease is rising to the point that such diseases are a major public health problem. Early treatment, which requires early diagnosis, is ideal. However, some tick-borne diseases, particularly Lyme disease and ehrlichiosis, are difficult to diagnose. As a result, the diseases are often missed and treatment early in the disease is not possible. There is an urgent need, thus, for new methods for the early diagnosis of tick-borne disease.

Another approach to the problem of tick-borne diseases is controlling the ticks. However, chemical control using acaricides poses significant problems for the environment and public health. In addition, ticks are developing resistance to the chemicals, making this approach also not effective. Accordingly, there is an urgent need for alternative methods for controlling tick infestation. One method utilizes host immunity to ticks. Tick immunity is the capacity of previously exposed hosts to interfere with tick feeding and development. A reduction in tick weight, duration of attachment, number of ticks feeding, size of egg mass and molting success are parameters to measure immunity. Tick immunity, induced by repeated tick exposure, has been shown in rabbits, cattle, dogs and guinea pigs [J. R. Allen, “Observations on the Behavior of Dermacentor andersoni Larvae Infesting Normal and Tick Resistant Guinea Pigs,” Parasitology, 84, pp. 195-204 (1982); M. Brossard et al., “Ixodes ricinus L.: Mast Cells, Basophils and Eosinophils in the Sequence of Cellular Events in the Skin of Infested or Re-infested Rabbits,” Parasitology, 85, pp. 583-592 (1982); Fivaz et al., “Cross-resistance Between Instars of the Brown Ear-tick Rhipicephalus appendiculatus (Acarina:Ixodidae),” Exp. Appl. Acarol., 11, pp. 323-326 (1991)]. Altered cytokine expression levels [Schorderet et al., “Effects of Human Recombinant Interleukin-2 on Resistance, and on the Humoral and Cellular Response of Rabbits Infested with Adult Ixodes ricinus Ticks,” Vet. Parasitol., 54pp. 375-387 (1994)], acute basophilic hypersensitivity at the site of the tick bite [Askenase et al., “Cutaneous Basophil-Associated Resistance to Ectoparasites (Ticks). I. Transfer with Immune Serum or Immune Cells,” Immunology, 45, pp. 501-511 (1982)], and circulating antibodies to several tick salivary gland proteins contribute to tick immunity [Wormser et al., “Requirement for Host Fc Receptors and IgG Antibodies in Host immune Responses Against Rhipicephalus appendiculatus,” Vet Parasitol., 28, pp. 153-l61 (1988); Girardin et al., “Effects of Cyclosporin A on Humoral Immunity to Ticks and on Cutaneous Immediate and Delayed Hypersensitivity Reactions to Ixodes ricinus L. Salivary-gland Antigens in Re-infested Rabbits,” Parasitol. Res., 75, pp. 657-662 (1989)].

The transmission of tick-borne pathogens, such as B. burgdorferi, the agent of Lyme disease, requires a prolonged period of feeding. If the feeding time can be shortened as a result of tick immunity, transmission of such tick-borne pathogens can be reduced.

Ixodid ticks are the most important arthropod vectors of infectious agents. Ixodes scapularis is the vector for Lyme disease, human granulocytic ehrlichiosis (HGE), babesia and tick-borne encephalitis. Accordingly, there is an urgent need to identify antigens of I. scapularis for use in inducing and detecting tick immunity.

### SUMMARY OF THE INVENTION

The present invention provides tick antigens for detecting and inducing tick immunity. One aspect of the invention provides compositions and methods for conferring and detecting tick immunity and for preventing or lessening the transmission of tick-borne pathogens. More particularly, this invention provides tick polypeptides, nucleic acid sequences encoding the polypeptides and antibodies (or antigen-binding portions thereof) specific for the polypeptides. The invention further provides compositions and methods comprising the polypeptides, nucleic acid sequences and antibodies.

In a preferred embodiment, the tick antigens are I. scapularis antigens.

Another aspect of the invention further provides a single or multicomponent pharmaceutical composition or vaccine comprising one or more tick antigens, preferably I. scapularis polypeptides, or antibodies of this invention.

A further aspect of the invention relates to nucleic acid molecules, including DNA, cDNA or RNA sequences that encode the tick antigens of the invention. The nucleic acid molecules of the invention include recombinant molecules comprising the nucleic acid molecules of the invention, unicellular hosts transformed with those nucleic acid sequences and molecules, and methods of using those sequences, molecules and hosts to produce tick polypeptides and vaccines comprising them. The nucleic acid molecules of the invention are advantageously used to make probes and polymerase chain reaction primers for use in isolating additional tick antigens.

Also within the scope of this invention are diagnostic means and methods characterized by a tick polypeptide or antibody of the invention. These means and methods are useful for the detection of tick bites and tick immunity. They are also useful in following the course of immunization against tick bites. In patients previously inoculated with the vaccines of this invention, the detection means and methods disclosed herein are also useful for determining if booster inoculations are appropriate.

Finally, this invention also provides methods for the identification and isolation of additional tick polypeptides, as well as compositions and methods comprising such polypeptides.

### BRIEF DESCRIPTION OF THE DRAWINGS

FIGS. 1A-1B are the DNA sequence of the I. scapularis salp25C gene (SEQ ID NO: 1).

FIG. 2 is the deduced amino-acid sequence of the I. scapularis Salp25C polypeptide (SEQ ID NO: 2).

FIGS. 3A-3B are the DNA sequence of the I. scapularis salp25D gene (SEQ ID NO: 3).

FIG. 4 is the deduced amino-acid sequence of the I. scapularis Salp25D polypeptide (SEQ ID NO: 4).

FIG. 5 is the DNA sequence of the I. scapularis salp14A gene (SEQ ID NO: 5).

FIG. 6 is the deduced amino-acid sequence of the I. scapularis Salp14A polypeptide (SEQ ID NO: 6).

FIG. 7 is the DNA sequence of the I. scapularis salp15 gene (SEQ ID NO: 7).

FIG. 8 is the deduced amino-acid sequence of the I. scapularis Salp15 polypeptide (SEQ ID NO: 8).

FIG. 9 is the DNA sequence of the I. scapularis salp 16A gene (SEQ ID NO: 9).

FIG. 10 is the deduced amino-acid sequence of the I. scapularis Salp16A polypeptide (SEQ ID NO: 10).

FIG. 11 is the DNA sequence of the I. scapularis salp17 gene (SEQ ID NO: 11).

FIG. 12 is the deduced amino-acid sequence of the I. scapularis Salp17 polypeptide (SEQ ID NO: 12).

FIG. 13 is the DNA sequence of the I. scapularis salp20 gene (SEQ ID NO: 13).

FIG. 14 is the deduced amino-acid sequence of the I. scapularis Salp20 polypeptide (SEQ ID NO: 14).

FIGS. 15A-15B are the DNA sequence of the I. scapularis salp25A gene (SEQ ID NO: 15).

FIG. 16 is the deduced amino-acid sequence of the I. scapularis Salp25A polypeptide (SEQ ID NO: 16).

FIGS. 17A-17B are the DNA sequence of the I. scapularis salp25B gene (SEQ ID NO: 17).

FIG. 18 is the deduced amino-acid sequence of the I. scapularis Salp25B polypeptide (SEQ ID NO: 18).

FIGS. 19A-19B are the DNA sequence of the I. scapularis salp26A gene (SEQ ID NO: 19).

FIG. 20 is the deduced amino-acid sequence of the I. scapularis Salp26A polypeptide (SEQ ID NO: 20).

FIGS. 21A-21B are the DNA sequence of the I. scapularis salp26B gene (SEQ ID NO: 21).

FIG. 22 is the deduced amino-acid sequence of the I. scapularis Salp26B polypeptide (SEQ ID NO: 22).

FIG. 23 is the DNA sequence of the I. scapularis salp14B gene (SEQ ID NO: 23).

FIG. 24 is the deduced amino-acid sequence of the I. scapularis Salp14B polypeptide (SEQ ID NO: 24).

FIG. 25 is the DNA sequence of the I. scapularis salp9 gene (SEQ ID NO: 25).

FIG. 26 is the deduced amino-acid sequence of the I. scapularis Salp9 polypeptide (SEQ ID NO: 26).

FIG. 27 is the DNA sequence of the I. scapularis salp10 gene (SEQ ID NO: 27).

FIG. 28 is the deduced amino-acid sequence of the I. scapularis Salp10 polypeptide (SEQ ID NO: 28).

FIG. 29 is the DNA sequence of the I. scapularis salp13 gene (SEQ ID NO: 29).

FIG. 30 is the deduced amino-acid sequence of the I. scapularis Salp13 polypeptide (SEQ ID NO: 30).

FIG. 31 is a graph of the number of ticks attached to control guinea pigs and guinea pigs immunized with Salp25C and Salp14B or by tick bite over time (hours).

FIG. 32 is a graph of engorged tick weights and percent survival of attached ticks on control guinea pigs and guinea pigs immunized with Salp25C and Salp14B or by tick bite.

FIG. 33A is the DNA and deduced amino-acid sequence of Salp15 (SEQ ID NO: 7 and SEQ ID NO: 8, respectively). The putative 22-amino-acid signal sequence is highlighted in grey. The amino-acid sequences highlighted in black represent regions that are homologous to Inhibin A fingerprints.

FIG. 33B shows an RT-PCR experiment with salp15 specific primers on mRNA obtained from unfed nymphs (U) and nymphs that had fed for 66 hours (F) showing upregulation of the gene upon feeding. Equal loading of cDNA was ensured with β-actin primers (control).

FIG. 33C shows an SDS-PAGE gel showing purified Salp15 as a thioredoxin (TR) fusion protein (lane 1) and TR (lane 2). Standard molecular weights in kDa are shown on the left.

FIG. 34A shows purified naive CD4⁺ T cells (10⁶/ml) which were activated in vitro with plate-bound anti-CD3 and soluble anti-CD28 in the presence of different concentrations of Salp15 (filled squares) or TR (open circles) for 44 hours.

FIG. 34B shows purified naive CD4⁺ T cells which were treated as in FIG. 34A except that Salp15 (filled squares) or TR (open circles) was held constant at 0.16 μM and IL-2 was measured at different time points. At the specified time points, IL-2 levels in the culture supernatants were measured by capture ELISA.

FIG. 34C shows 10⁶ naive CD4⁺ T cells which were activated with anti-CD3 and anti-CD28 in the presence of I. scapularis saliva (dilutions of 1:50, 1:100 and 1:500). As a control, 10⁶ naive CD4⁺ T cells were activated with anti-CD3 and anti-CD28 for 44 hours in the absence of tick saliva (Control), and IL-2 levels were measured as for FIG. 34B.

FIG. 34D shows naive CD4⁺ T cells (10⁶/ml) which were activated with anti-CD3/CD28 for 44 hours in the presence of 0.32 μM Salp15 (grey line) or TR (black line). CD25 and CD25 expression was analyzed by flow cytometry.

FIG. 34E shows naive CD4⁺ T cells (10⁶/ml) which were activated with anti-CD3/CD28 for 44 hours in the presence of different concentrations of Salp15 (filled squares) or TR (open circles). CD25 expression was then analyzed by flow cytometry.

FIG. 34F shows 10⁵ naive CD4⁺ T cells which were activated with anti-CD3/CD28 for 78 hours in the presence of different concentrations of Salp15 (filled squares) or TR (open circles). Cells were pulsed with 1 μCi [³H]-thymidine for the last 18 hours of the assay. The results shown are representative of 4 individual experiments

FIG. 35A shows 10⁵ naive CD4⁺ T cells which were activated with anti-CD3/CD28 in the presence of equimolar concentrations of Salp15 or TR in the presence of medium (white bars) or exogenous recombinant murine IL-2 (10 ng/ml) (black bars). At 60 hours of activation, the cells were pulsed with [³H]-thymidine for 18 hours.

FIG. 35B shows 10⁶ naive CD4⁺ T cells which were activated with anti-CD3/CD28. At 12 (white bars) or 24 (black bars) hours of activation, equimolar concentrations of Salp15 or TR were added to the cultures. The levels of IL-2 in the culture supernatants were determined by capture ELISA at 44 hours of activation. The hatched bar represents the level of IL-2 produced by the cells prior to the addition of Salp15 at 24 hours. The results shown are representative of 2 independent experiments.

FIG. 35C shows that Salp15 inhibits AP-1, NF-κB and predominantly NF-AT DNA binding activity. Nuclear extracts were obtained from 12-hour anti-CD3/CD28 activated naive CD4⁺ T cells in the presence of thioredoxin (1) or Salp15 (2). Electromobility shift analysis was then performed using 2 μg of the extracts and ³²P end-labeled double-stranded oligonucleotides representing the consensus binding sites for AP-1, NF-AT and NF-κB.

FIG. 36A shows that Salp15 inhibits CD4⁺ T cell activation in vivo. Groups of 4 Balb/c mice were immunized with equimolar quantities of TR or Salp15 fused to TR. Eleven days later, CD4⁺ T cells were purified and analyzed in recall responses to TR (black bars) and Salp15 (white bars). IFN-γ levels were measured by capture ELISA in 40-hour restimulation supernatants.

FIG. 36B shows TR-specific IgM (black bars, 1:160 dilution) and IgG (white bars, 1:320 dilution) levels in the sera of the immunized mice which were determined by ELISA using TR-bound plates. Sera from unimmunized mice were used as a control (NMS). These data represent the mean and standard deviation of 4 mice in each group.

FIG. 37A shows host immunity against ticks in guinea pigs. Fifty I. scapularis nymphs were placed on naive (control), tick-immune and animals that were passively immunized with tick-immune sera. There were at least 3 animals in each group. The duration of tick attachment was recorded from experimental and control guinea pigs. An (*) mark on each data point denotes a statistically significant difference at least at the level of P<0.05 (Student's t-test).

FIG. 37B shows host immunity against ticks in rabbits. Fifty I. scapularis nymphs were placed on naive (control), tick-immune and animals that were passively immunized with tick-immune sera. There were at least 3 animals in each group. The duration of tick attachment was recorded from experimental and control rabbits. An (*) mark on each data point denotes a statistically significant difference at least at the level of P<0.05 (Student's t-test).

FIG. 37C shows host immunity against ticks in guinea pigs. Fifty I. scapularis nymphs were placed on naive (control), tick-immune and animals that were passively immunized with tick-immune sera. There were at least 3 animals in each group and the weight of recovered ticks was recorded from experimental and control guinea pigs. An (*) mark on each data point denotes a statistically significant difference at least at the level of P<0.05 (Student's t-test). The severity of erythema © and D) was measured on a scale from 0 to 3, where 0 represents a lack of erythema and 1, 2, and 3 represent mild, moderate and severe erythema.

FIG. 37D shows host immunity against ticks in rabbits. Fifty I. scapularis nymphs were placed on naive (control), tick-immune and animals that were passively immunized with tick-immune sera. There were at least 3 animals in each group and the weight of recovered ticks was recorded from experimental and control rabbits. An (*) mark on each data point denotes a statistically significant difference at least at the level of P<0.05 (Student's t-test). The severity of erythema was measured on a scale from 0 to 3, where 0 represents a lack of erythema and 1, 2, and 3 represent mild, moderate and severe erythema.

FIG. 38A shows a silver-stained gel of extracts of 50 tick salivary glands that were analyzed by 2-D gel electrophoresis. An (*) mark denotes the spot which was cut out of the gel for peptide sequencing.

FIG. 38B shows the proteins separated as in FIG. 38A which were then transferred to a nylon membrane and probed with tick-immune rabbit sera. An (*) mark denotes the spot corresponding to the spot in FIG. 38A which was recognized by tick-immune rabbit sera.

FIG. 38C shows the proteins separated as in FIG. 38A which were then transferred to a nylon membrane and probed with normal rabbit sera.

FIG. 39 shows the analysis of the mRNAs encoding 14 of the salivary antigens, that were identified by immunoscreening, in the salivary glands from unengorged and engorged I. scapularis nymphs. Total RNA was isolated from freshly dissected salivary glands of unfed and 66 hr-fed nymphs, and cDNA was made using oligo-dT primer and reverse transcriptase (RT). Equal amounts of cDNA (2 μg) was used as the template for PCR with specific pair of primers for each gene. Samples without (−) and with (+) RT were used to determine whether genomic DNA was present.

FIG. 40 shows an amino-acid sequence alignment of Salp25C with cow, human and nematode glutathione peroxidases. Identical and similar amino acids are designated in capital letters, different amino acids are in lowercase letters and gaps are designated with “−”.

FIG. 41 shows the expression and purification of recombinant Salp25C as a thioredoxin fusion protein. Salp25C was inserted in-frame with the thioredoxin gene and expression was induced with arabinose. Cells were sonicated and the soluble fraction (supernatant) was used to purify the protein by affinity column chromatography. (I), Supernatant from lysed, induced cells; (U), supernatant from lysed, uninduced cells; (P), purified protein.

FIG. 42 shows glutathione peroxidase activity. A positive control of bovine glutathione peroxidase (GPX, 100 nmol/ml), and a negative control of thioredoxin (TR, 330 nmol/ml) were also assayed and compared to that of thioredoxin-Salp25C fusion protein (TR-Salp25C, 330, 660 and 1000 nmol/ml).

FIG. 43 shows the anticoagulant activity of Salp14A.

### DETAILED DESCRIPTION OF THE INVENTION

This invention relates to tick polypeptides and nucleic acid sequences encoding them, antibodies directed against those polypeptides, compositions comprising the polypeptides, nucleic acids or antibodies. This invention further relates to methods for identifying additional tick polypeptides and antibodies and methods for conferring and detecting tick immunity and for preventing or lessening the transmission of tick-borne pathogens.

In preferred embodiments, this invention provides fifteen novel I. scapularis polypeptides and compositions and methods comprising the polypeptides. More specifically, this invention provides a Salp9 polypeptide, a Salp10 polypeptide, a Salp13 polypeptide, a Salp14A polypeptide, a Salp14B polypeptide, a Salp15 polypeptide, a Salp16A polypeptide, a Salp17 polypeptide, a Salp20 polypeptide, a Salp25A polypeptide, a Salp25B polypeptide, a Salp25C polypeptide, a Salp25D polypeptide, a Salp26A polypeptide, and a Salp26B polypeptide.

Also within the scope of the invention are polypeptides that are at least 75% homologous in amino-acid sequence to the aforementioned polypeptides. In preferred embodiments, the polypeptides are at least 80%, 85%, 90% or 95% homologous in amino-acid sequence to an amino-acid sequence set forth herein. In more preferred embodiments, the homologous polypeptides have the biological activity or activities of the tick polypeptides of the invention.

Also within the scope of the invention are fragments of the above-listed polypeptides. The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The preferred compositions and methods of each of the aforementioned embodiments are characterized by immunogenic polypeptides. As used herein, an “immunogenic polypeptide” is a polypeptide that, when administered to an animal, is capable of eliciting a corresponding antibody.

The most preferred compositions and methods of each of the aforementioned embodiments are characterized by I. scapularis polypeptides that elicit, in treated animals, the formation of a tick immune response. As used herein, a “tick immune response” or “tick immunity” is manifested by one or more of the following: reduction in the duration of tick attachment to a host, a reduction in the weight of ticks recovered after detaching from the host compared to those values in ticks that attach to non-immune hosts, failure of the ticks to complete their development and failure to lay the normal number of viable eggs.

In another preferred embodiment, this invention provides a vaccine comprising one or more tick polypeptides, preferably one or more I. scapularis polypeptides of this invention or one or more antibodies directed against a polypeptide of this invention.

As used herein, a substantially pure polypeptide is a polypeptide that is detectable as a single band on an immunoblot probed with polyclonal anti-serum.

A further embodiment of the invention are polynucleotides, including DNA, cDNA and RNA, encoding a polypeptide of the invention. More specifically, the invention includes fifteen novel DNA molecules encoding the I. scapularis polypeptides of the invention. In particular, the invention provides a DNA molecule comprising the DNA sequence encoding a Salp9 polypeptide, a Salp10 polypeptide, a Salp13 polypeptide, a Salp14A polypeptide, a Salp14B polypeptide, a Salp15 polypeptide, a Salp16A polypeptide, a Salp17 polypeptide, a Salp20 polypeptide, a Salp25A polypeptide, a Salp25B polypeptide, a Salp25C polypeptide, a Salp25D polypeptide, a Salp26A polypeptide, and a Salp26B polypeptide, as set forth in the Figures.

The invention also relates to polynucleotides that hybridize to the above-described polynucleotides and differ at one or more positions in comparison to these as long as they encode a tick polypeptide as defined above. Such molecules comprise those which are changed, for example, by nucleotide deletion(s), insertion(s), alteration(s) or any other modification known in the art in comparison to the above-described polynucleotides either alone or in combination. Methods for introducing such modifications in the polynucleotides of the invention are well-known to the person skilled in the art; see, e.g., Sambrook et al. [Molecular cloning; A Laboratory Manual, Second Edition, Cold Spring Harbor Laboratory Press, Cold Spring Harbor NY (1989)]. The invention also relates to polynucleotides the nucleotide sequence of which differs from the nucleotide sequence of any of the above-described polynucleotides due to the degeneracy of the genetic code.

The term “hybridizing” in this context is understood as referring to conventional hybridization conditions, such as hybridization in 50% formamide; 6×SSC; 0.1% SDS; 100 μg/ml ssDNA, in which temperatures for hybridization are above 37° C. and temperatures for washing in 0.1×SSC; 0.1% SDS are above 55° C. The term “hybridizing” refers to stringent hybridization conditions, for example such as described in Sambrook, supra.

Nucleic-acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art.

“Stringent hybridization conditions” and “stringent wash conditions” in the context of nucleic-acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, “stringent hybridization” is performed at about 25° C. below the thermal melting point (Tₘ) for the specific nucleic acid hybrid under a particular set of conditions. “Stringent washing” is performed at temperatures about 5° C. lower than the Tₘ for the specific nucleic acid hybrid under a particular set of conditions. The Tₘ is the temperature at which 50% of the target sequence hybridizes to a perfectly matched probe. See Sambrook et al., page 9.51, hereby incorporated by reference.

The Tₘ for a particular DNA-DNA hybrid can be estimated by the formula:

Tₘ=81.5° C.+16.6(log₁₀[Na⁺])+0.41(fraction G+C)−0.63(% formamide)−(600/l) where l is the length of the hybrid in base pairs.

The Tₘ for a particular RNA-RNA hybrid can be estimated by the formula:

Tₘ=79.8° C.+18.5(log₁₀[Na⁺])+0.58(fraction G+C)+11.8(fraction G+C)2−0.35(% formamide)−(820/l).

The Tₘ for a particular RNA-DNA hybrid can be estimated by the formula:

Tₘ=79.8° C.+18.5(log₁₀[Na⁺])+0.58(fraction G+C)+11.8(fraction G+C)2−0.50 (% formamide)−(820/l).

In general, the Tₘ decreases by 1-1.5° C. for each 1% of mismatch between two nucleic-acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic-acid sequence, 10-15° C. would be subtracted from the calculated Tₘ of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well-known in the art.

An example of stringent hybridization conditions for hybridization of complementary nucleic-acid sequences having more than 100 complementary residues on a filter in a Southern or northern blot or for screening a library is 50% formamide;6×SSC at 42° C. for at least ten hours. Another example of stringent hybridization conditions is 6×SSC at 68° C. for at least ten hours. An example of low stringency hybridization conditions for hybridization of complementary nucleic-acid sequences having more than 100 complementary residues on a filter in a Southern or northern blot or for screening a library is 6×SSC at 42° C. for at least ten hours. Hybridization conditions to identify nucleic-acid sequences that are similar but not identical can be identified by experimentally changing the hybridization temperature from 68° C. to 42° C. while keeping the salt concentration constant (6×SSC), or keeping the hybridization temperature and salt concentration constant (e.g. 42° C. and 6×SSC) and varying the formamide concentration from 50% to 0%. Hybridization buffers may also include blocking agents to lower background. These agents are well-known in the art. See Sambrook et al., supra, pages 8.46 and 9.46-9.58, herein incorporated by reference. Wash conditions also can be altered to change stringency conditions. An example of stringent wash conditions is an 0.2×SSC wash at 65° C. for 15 minutes (see Sambrook et al., supra, for SSC buffer). Often the high stringency wash is preceded by a low stringency wash to remove excess probe. An exemplary medium stringency wash for duplex DNA of more than 100 base pairs is 1×SSC at 45° C. for 15 minutes. An exemplary low stringency wash for such a duplex is 4×SSC at 40° C. for 15 minutes. In general, a signal-to-noise ratio of 2× or higher than that observed for an unrelated probe in the particular hybridization assay indicates detection of a specific hybridization.

Particularly preferred are polynucleotides that share 75%, preferably at least 85%, more preferably 90-95%, and most preferably 96-99% sequence identity with one of the above-mentioned polynucleotides and encode a polypeptide having the same biological activity. Thus, the present invention encompasses any polynucleotide that can be derived from the above-described polynucleotides by way of genetic engineering and that encode upon expression a tick polypeptide of the invention or a fragment thereof.

The term “percent sequence identity” or “identical” in the context of nucleic-acid sequences refers to the residues in the two sequences which are the same when aligned for maximum correspondence. There are a number of different algorithms known in the art which can be used to measure nucleotide sequence identity. For instance, polynucleotide sequences can be compared using NCBI BLASTx and BLASTn software. Alternatively, Fasta, a program in GCG Version 6.1. Fasta provides alignments and percent sequence identity of the regions of the best overlap between the query and search sequences (Pearson, 1990). For instance, percent sequence identity between nucleic-acid sequences can be determined using Fasta with its default parameters (a word size of 6 and the NOPAM factor for the scoring matrix) as provided in GCG Version 6.1, herein incorporated by reference.

In yet another embodiment, this invention provides antibodies or an antigen-binding portion thereof,that specifically bind a polypeptide of this invention, and pharmaceutically effective compositions and methods comprising those antibodies. The antibodies of this invention are those that are reactive with a polypeptide, preferably an I. scapularis polypeptide of this invention. Such antibodies may be used in a variety of applications, including to detect expression of tick antigens, preferably I. scapularis antigens, to screen for expression of novel tick polypeptides, to purify novel tick polypeptides and to confer tick immunity. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide.

In still another embodiment, this invention relates to diagnostic means and methods characterized by a tick polypeptide or immunogenic fragment thereof, nucleic acid sequence, antibody or antigen-binding fragment thereof of the invention.

A further embodiment of this invention provides methods for inducing tick immunity in a host by administering one or more tick polypeptides, preferably I. scapularis polypeptides, or one or more antibodies of the invention.

A preferred embodiment of this invention is a method for preventing or reducing the transmission of tick-borne pathogens by administering polypeptides or antibodies of this invention that are effective to induce tick immunity. A particularly preferred embodiment is a method for preventing or reducing the severity for some period of time of B. burgdorferi infection.

In order to further define this invention, the following terms and definitions are herein provided.

As used herein, an “I. scapularis polypeptide” T is a polypeptide encoded by a DNA sequence of I. scapularis . For example, I. scapularis polypeptides include but are not limited to the Salp9 polypeptide, Salp10 polypeptide, Salp13 polypeptide, Salp14A polypeptide, Salp14B polypeptide, Salp15 polypeptide, Salp16A polypeptide, Salp17 polypeptide, Salp20 polypeptide, Salp25A polypeptide, Salp25B polypeptide, Salp25C polypeptide, Salp25D polypeptide, Salp26A polypeptide, and Salp26B polypeptide provided herein, and fragments or derivatives thereof.

As used herein, a “protective tick polypeptide” is a tick polypeptide that, when administered to an animal, elicits an immune response that is effective to confer tick immunity or to prevent or lessen the severity, for some period of time, of infection by a tick-borne pathogen. Preventing or lessening the severity of infection may be evidenced by a change in the physiological manifestations of infection with that pathogen. In a preferred embodiment, the tick-borne pathogen is B. burgdorferi, and preventing or lessening the severity of infection includes preventing or lessening the severity of erythema migrans, arthritis, carditis, neurological disorders, and other Lyme disease-related disorders. Prevention may be evidenced by a decrease in or absence of spirochetes in the treated animal and it may be evidenced by a decrease in the level of spirochetes in infected ticks which have fed on treated animals.

One of skill in the art will understand that probes and oligonucleotide primers derived from a nucleic acid molecule encoding a tick polypeptide of the invention may be used to isolate and clone variants of polypeptides from other Ixodes isolates and perhaps from other-hard-bodied ticks as well. Such variants are useful in the methods and compositions of this invention.

As used herein, a “derivative” of a tick polypeptide is a polypeptide in which the native form has been modified or altered. Such modifications include, but are not limited to: amino-acid substitutions, modifications, additions or deletions; alterations in the pattern of lipidation, glycosylation or phosphorylation; reactions of free amino, carboxyl, or hydroxyl side groups of the amino-acid residues present in the polypeptide with other organic and non-organic molecules; and other modifications, any of which may result in changes in primary, secondary or tertiary structure.

As used herein, a “protective epitope” is (1) an epitope that is recognized by a protective antibody, and/or (2) an epitope that, when used to immunize an animal, elicits an immune response sufficient to confer tick immunity or to prevent or lessen the severity for some period of time, of infection with a tick-borne pathogen. A protective epitope may comprise a T-cell epitope, a B-cell epitope, or combinations thereof.

As used herein, a “protective antibody” is an antibody that confers tick immunity or protection for some period of time, against infection by a tick-borne pathogen or any one of the physiological disorders associated with such infection. In a preferred embodiment, the antibody confers protection against B. burgdorferi infection.

As used herein, a “T-cell epitope” is an epitope which, when presented to T cells by antigen-presenting cells, results in a T-cell response such as clonal expansion or expression of lymphokines or other immunostimulatory molecules. A strong T-cell epitope is a T-cell epitope which elicits a strong T-cell response.

As used herein, a “B-cell epitope” is the simplest spatial conformation of an antigen which reacts with a specific antibody.

As used herein, a “therapeutically effective amount” of a polypeptide or of an antibody is the amount that, when administered to an animal, elicits an immune response that is effective to confer tick immunity or to prevent or lessen the severity, for some period of time, of infection by a tick-borne pathogen.

An antibody of this invention includes antibodies directed against a tick polypeptide or a fragment, derivative or variant thereof, preferably a polypeptide expressed by I. scapularis , or an antigen-binding fragment or derivative thereof that are immunologically cross-reactive with any one of the aforementioned polypeptides. Finally, an antibody of this invention includes antibodies directed against other tick polypeptides, preferably I. scapularis polypeptides, identified according to methods taught herein.

As used herein, an antibody is an immunoglobulin molecule, or antigen-binding portion thereof, that is immunologically reactive with a tick polypeptide of the present invention, wherein that immunoglobulin molecule was either elicited by immunization with a tick or a tick polypeptide of this invention or was isolated or identified by its reactivity with a polypeptide of this invention.

An antibody of this invention may be an intact immunoglobulin molecule or an antigen-binding fragment of an immunoglobulin molecule, including fragments and single-chain F(v). It should be understood that an antibody of this invention may also be a protective antibody.

The I. scapularis polypeptides disclosed herein are immunologically reactive with antisera generated by immunization with I. scapularis extracts or by tick bite. Accordingly, they are useful in methods and compositions to detect tick immunity.

The I. scapularis polypeptides disclosed herein are are particularly useful in single and multicomponent vaccines against tick bites and infection by tick-borne pathogens. In this regard, multicomponent vaccines are preferred because such vaccines may be formulated to more closely resemble the immunogens presented by tick bite, and because such vaccines are more likely to confer broad-spectrum protection than a vaccine comprising only a single tick polypeptide.

Multicomponent vaccines according to this invention may also contain polypeptides that characterize other vaccines useful for immunization against diseases such as, for example, Lyme disease, human monocytic ehrlichiosis, babesiosis, diphtheria, polio, hepatitis, and measles. Such multicomponent vaccines are typically incorporated into a single composition.

The preferred compositions and methods of this invention comprise I. scapularis polypeptides having enhanced immunogenicity. Such polypeptides may result when the native forms of the polypeptides or fragments thereof are modified or subjected to treatments to enhance their immunogenic character in the intended recipient.

Numerous techniques are available and well-known to those of skill in the art which may be used, without undue experimentation, to substantially increase the immunogenicity of the tick polypeptides herein disclosed. For example, a tick polypeptide of this invention may be modified by coupling to dinitrophenol groups or arsanilic acid, or by denaturation with heat and/or SDS. Particularly if the polypeptides are small, chemically synthesized polypeptides, it may be desirable to couple them to an immunogenic carrier. The coupling, of course, must not interfere with the ability of either the polypeptide or the carrier to function appropriately. For a review of some general considerations in coupling strategies, see Antibodies, A Laboratory Manual, Cold Spring Harbor Laboratory, ed. E. Harlow and D. Lane (1988).

Useful immunogenic carriers are well-known in the art. Examples of such carriers are keyhole limpet hemocyanin (KLH); albumins such as bovine serum albumin (BSA) and ovalbumin, PPD (purified protein derivative of tuberculin); red blood cells; tetanus toxoid; cholera toxoid; agarose beads; activated carbon; or bentonite.

Modification of the amino-acid sequence of the polypeptides disclosed herein to alter the lipidation state is also a method which may be used to increase their immunogenicity or alter their biochemical properties. For example, the polypeptides or fragments thereof may be expressed with or without the signal and other sequences that may direct addition of lipid moieties.

As will be apparent from the disclosure to follow, the polypeptides may also be prepared with the objective of increasing stability or rendering the molecules more amenable to purification and preparation. One such technique is to express the polypeptides as fusion proteins comprising other tick, preferably I. scapularis , or non-I. scapularis sequences.

In accordance with this invention, a derivative of a polypeptide of the invention may be prepared by a variety of methods, including by in vitro manipulation of the DNA encoding the native polypeptides and subsequent expression of the modified DNA, by chemical synthesis of derivatized DNA sequences, or by chemical or biological manipulation of expressed amino-acid sequences.

For example, derivatives may be produced by substitution of one or more amino acids with a different natural amino acid, an amino-acid derivative or non-native amino acid. Those of skill in the art will understand that conservative substitution is preferred, e.g., 3-methylhistidine may be substituted for histidine, 4-hydroxyproline may be substituted for proline, 5-hydroxylysine may be substituted for lysine, and the like.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well-known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). See also, Creighton (1984) Proteins W. H. Freeman and Company.

Conservative substitutions typically include the substitution of one amino acid for another with similar characteristics such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

Other conservative substitutions are described by Dayhoff in the Atlas of Protein Sequence and Structure, (1988).

Causing amino-acid substitutions which are less conservative may also result in desired derivatives, e.g., by causing changes in charge, conformation and other biological properties. Such substitutions would include for example, substitution of a hydrophilic residue for a hydrophobic residue, substitution of a cysteine or proline for another residue, substitution of a residue having a small side chain for a residue having a bulky side chain or substitution of a residue having a net positive charge for a residue having a net negative charge.

When the result of a given substitution cannot be predicted with certainty, the derivatives may be readily assayed according to the methods disclosed herein to determine the presence or absence of the desired characteristics. In particular, the immunogenicity, immunodominance and/or protectiveness of a derivative of this invention can be readily determined using methods disclosed in the Examples.

In a preferred embodiment of this invention, the I. scapularis polypeptides disclosed herein are prepared as part of a larger fusion protein. For example, an I. scapularis polypeptide of this invention may be fused at its N-terminus or C-terminus to a different immunogenic I. scapularis polypeptide, to a non-I. scapularis polypeptide or to combinations thereof, to produce fusion proteins comprising the I. scapularis polypeptide.

In a preferred embodiment of this invention, fusion proteins comprising a tick polypeptide, preferably an I. scapularis polypeptide, are constructed comprising B-cell and/or T-cell epitopes from multiple serotypic variants of I. scapularis , each variant differing from another with respect to the locations or sequences of the epitopes within the polypeptide. In a more preferred embodiment, fusion proteins are constructed which comprise one or more of the I. scapularis polypeptides fused to other I. scapularis polypeptides. Such fusion proteins are particularly effective in the induction of tick immunity against a wide spectrum of isolates.

In another preferred embodiment of this invention, the I. scapularis polypeptides are fused to moieties, such as immunoglobulin domains, which may increase the stability and prolong the in vivo plasma half-life of the polypeptide. Such fusions may be prepared without undue experimentation according to methods well-known to those of skill in the art, for example, in accordance with the teachings of U.S. Pat. No. 4,946,778, or U.S. Pat. No. 5,116,964. The exact site of the fusion is not critical as long as the polypeptide retains the desired biological activity. Such determinations may be made according to the teachings herein or by other methods known to those of skill in the art.

It is preferred that the fusion proteins comprising the I. scapularis polypeptides be produced at the DNA level, e.g., by constructing a nucleic-acid molecule encoding the fusion protein, transforming host cells with the molecule, inducing the cells to express the fusion protein, and recovering the fusion protein from the cell culture. Alternatively, the fusion proteins may be produced after gene expression according to known methods.

The polypeptides of the invention may also be part of larger multimeric molecules which may be produced recombinantly or may be synthesized chemically. Such multimers may also include the polypeptides fused or coupled to moieties other than amino acids, including lipids and carbohydrates.

Preferably, the multimeric proteins will consist of multiple T- or B-cell epitopes or combinations thereof repeated within the same molecule, either randomly, or with spacers (amino-acid or otherwise) between them.

In a preferred embodiment of this invention, one or more I. scapularis antigens or polypeptides of the invention are incorporated into a vaccine. As described in Example V, the duration time of attachment and feeding of ticks exposed to animals that are immunized with a tick antigen of the invention is reduced. Without being confined to any particular mechanism of action for such a vaccine, it is believed that antibodies generated in an immunized host against tick polypeptides can form complexes that alone or in association with immune cells or serum factors, disturb or block the feeding of ticks. Alternatively or additionally, such antibodies generated in the immunized host cause an irritation at or near the tick attachment site, such irritation resulting in a reduction in the duration of tick attachment or feeding. As all of the tick antigens of the invention were identified using antibodies in sera from animals exposed to tick bites, they are all known to be immunogenic and, thus, useful in the above-described vaccines.

Another possible mechanism of action of a vaccine comprising tick antigens of the invention is the generation of antibodies that neutralize anti-complement proteins in tick saliva. As complement plays a role in producing tick-immunity, anti-complement proteins inhibit tick immunity, allowing ticks to feed repeatedly on the same host. Neutralizing anti-complement proteins, thus, enhances tick immunity. Salp 20, for example, is approximately 80% homologous to an I. scapularis polypeptide that has been reported to have anti-complement activity [Valenzuela et al., “Purification , Cloning and Expression of a Novel Salivary Anticomplement Protein from the Tick, Ixodes scapularis”, J. Biol. Chem., 275, pp. 18717-23 (June 2000)].

In another preferred embodiment of this invention, a tick polypeptide of this invention, preferably an I. scapularis polypeptide is incorporated into a single-component vaccine. In a more preferred embodiment of this invention, I. scapularis polypeptides of this invention, are incorporated into a multicomponent vaccine comprising other protective tick polypeptides. In addition, a multicomponent vaccine may also contain protective polypeptides useful for immunization against other diseases such as, for example, Lyme disease, human monocytic ehrlichiosis, babesiosis, diphtheria, polio, hepatitis, and measles. Such a vaccine, by virtue of its ability to elicit antibodies to a variety of protective I. scapularis polypeptides, will be effective to protect against tick bite by a broad spectrum of ticks, even those that may not express one or more of the I. scapularis proteins.

The multicomponent vaccine may contain a tick polypeptide as part of a multimeric molecule in which the various components are covalently associated. Alternatively, it may contain multiple individual components. For example, a multicomponent vaccine may be prepared comprising two or more of the I. scapularis polypeptides, wherein each polypeptide is expressed and purified from independent cell cultures and the polypeptides are combined prior to or during formulation.

Alternatively, a multicomponent vaccine may be prepared from heterodimers or tetramers wherein the polypeptides have been fused to immunoglobulin chains or portions thereof. Such a vaccine could comprise, for example, a Salp25C polypeptide fused to an immunoglobulin heavy chain and a Salp14B polypeptide, fused to an immunoglobulin light chain, and could be produced by transforming a host cell with DNA encoding the heavy-chain fusion and DNA encoding the light-chain fusion. One of skill in the art will understand that the host cell selected should be capable of assembling the two chains appropriately. Alternatively, the heavy- and light-chain fusions could be produced from separate cell lines and allowed to associate after purification.

The desirability of including a particular component and the relative proportions of each component may be determined by using the assay systems disclosed herein, or by using other systems known to those in the art. Most preferably, the multicomponent vaccine will comprise numerous T-cell and B-cell epitopes of protective I. scapularis polypeptides.

This invention also contemplates that a tick polypeptide, preferably an I. scapularis polypeptide of this invention, either alone or combined, may be administered to an animal via a liposome delivery system in order to enhance their stability and/or immunogenicity. Delivery of an I. scapularis polypeptide via liposomes may be particularly advantageous because the liposome may be internalized by phagocytic cells in the treated animal. Such cells, upon ingesting the liposome, would digest the liposomal membrane and subsequently present the polypeptide to the immune system in conjunction with other molecules required to elicit a strong immune response.

The liposome system may be any variety of unilamellar vesicles, multilamellar vesicles, or stable plurilamellar vesicles, and may be prepared and administered according to methods well-known to those of skill in the art, for example in accordance with the teachings of U.S. Pat. Nos. 4,762,915, 5,000,958, 5,169,637 or 5,185,154. In addition, it may be desirable to express the I. scapularis polypeptides of this invention, as well as other selected I. scapularis polypeptides, as lipoproteins, in order to enhance their binding to liposomes.

Any of the polypeptides of this invention may be used in the form of a pharmaceutically acceptable salt. Suitable acids and bases which are capable of forming salts with the polypeptides of the present invention are well-known to those of skill in the art, and include inorganic and organic acids and bases.

According to this invention, we describe a method which comprises the steps of treating an animal with a therapeutically effective amount of a tick polypeptide, preferably an I. scapularis polypeptide, or a fusion protein or a multimeric protein comprising an I. scapularis polypeptide, in a manner sufficient to confer tick immunity or prevent or lessen the severity, for some period of time, of infection by a tick-borne pathogen. The polypeptides that are preferred for use in such methods are those that contain protective epitopes. Such protective epitopes may be B-cell epitopes, T-cell epitopes, or combinations thereof.

According to another embodiment of this invention, we describe a method which comprises the steps of treating an animal with a multicomponent vaccine comprising a therapeutically effective amount of a tick polypeptide or immunogenic fragment thereof, or a fusion protein or multimeric protein comprising such polypeptide or fragment thereof in a manner sufficient to confer tick immunity or prevent or lessen the severity, for some period of time, of infection by a tick-borne pathogen. Again, the polypeptides, fusion proteins and multimeric proteins that are preferred for use in such methods are those that contain protective epitopes, which may be B-cell epitopes, T-cell epitopes, or combinations thereof.

The most preferred polypeptides, fusion proteins and multimeric proteins for use in these compositions and methods are those containing both strong T-cell and B-cell epitopes. Without being bound by theory, we believe that this is the best way to stimulate high-titer antibodies that are effective to confer tick immunity. Such preferred polypeptides will be internalized by B cells expressing surface immunoglobulin that recognizes the B-cell epitope(s). The B cells will then process the antigen and present it to T cells. The T cells will recognize the T-cell epitope(s) and respond by proliferating and producing lymphokines which in turn cause B cells to differentiate into antibody-producing plasma cells. Thus, in this system, a closed autocatalytic circuit exists which will result in the amplification of both B- and T-cell responses, leading ultimately to production of a strong immune response which includes high-titer antibodies against the I. scapularis polypeptide.

One of skill in the art will also understand that it may be advantageous to administer a polypeptide of this invention in a form that will favor the production of T-helper cells type 1 (T_{H}1), which help activate macrophages, and/or T-helper cells type 2 (T_{H}2), which help B cells to generate antibody responses. Aside from administering epitopes which are strong T-cell or B-cell epitopes, the induction of T_{H}1 or T_{H}2 cells may also be favored by the mode of administration of the polypeptide. For example, an I. scapularis polypeptide may be administered in certain doses or with particular adjuvants and immunomodulators, for example with interferon-gamma or interleukin-12 (T_{H}1 response) or interleukin-4 or interleukin-10 (T_{H}2 response)

To prepare the preferred polypeptides and fragments of this invention, in one embodiment, overlapping fragments of the I. scapularis polypeptides of this invention are constructed. The polypeptides that contain B-cell epitopes may be identified in a variety of ways for example by their ability to (1) remove protective antibodies from polyclonal antiserum directed against the polypeptide or (2) elicit an immune response which is effective to confer tick immunity.

Alternatively, a tick polypeptide or an immunogenic fragment thereof may be used to produce monoclonal antibodies that are screened for their ability to confer tick immunity when used to immunize naive animals. Once a given monoclonal antibody is found to confer protection, the particular epitope that is recognized by that antibody may then be identified.

As recognition of T-cell epitopes is MHC-restricted, the polypeptides that contain T-cell epitopes may be identified in vitro by testing them for their ability to stimulate proliferation and/or cytokine production by T-cell clones generated from humans of various HLA types, from the lymph nodes, spleens, or peripheral blood lymphocytes of C3H or other laboratory mice, or from domestic animals. Compositions comprising multiple T-cell epitopes recognized by individuals with different Class II antigens are useful for prevention and treatment of human granulocytic ehrlichiosis in a broad spectrum of patients.

In a preferred embodiment of the present invention, a tick polypeptide or fragment thereof, preferably an I. scapularis polypeptide or fragment, containing a B-cell epitope is fused to one or more other immunogenic I. scapularis polypeptides containing strong T-cell epitopes. The fusion protein that carries both strong T-cell and B-cell epitopes is able to participate in elicitation of a high-titer antibody response effective to confer tick immunity.

Strong T-cell epitopes may also be provided by non-I. scapularis molecules. For example, strong T-cell epitopes have been observed in hepatitis B virus core antigen (HBcAg). Furthermore, it has been shown that linkage of one of these segments to segments of the surface antigen of Hepatitis B virus, which are poorly recognized by T cells, results in a major amplification of the anti-HBV surface antigen response, [D. R. Milich et al., “Antibody Production to the Nucleocapsid and Envelope of the Hepatitis B Virus Primed by a Single Synthetic T Cell Site”, Nature, 329, pp. 547-49 (1987)].

Therefore, in yet another preferred embodiment, B-cell epitopes of the I. scapularis polypeptides are fused to segments of HBcAG or to other antigens which contain strong T-cell epitopes, to produce a fusion protein that can elicit a high-titer antibody response against I. scapularis antigens. In addition, it may be particularly advantageous to link an I. scapularis polypeptide of this invention to a strong immunogen that is also widely recognized, for example tetanus toxoid.

The tick antigens of the invention have additional uses. We demonstrate for example, that Salp 25C, is a glutathione peroxidase. Glutathione peroxidase is involved in a biochemical pathway that removes oxidative radicals produced, for example, as a result of inflammation. We also have demonstrated that Salp15 inhibits IL-2 and CD25 production and T cell proliferation. Further, Salp13 shows some homology to TGF-β family members and, thus, also may have anti-inflammatory activity.

Accordingly, Salp25C, Salp15 and Salp 13 are useful to inhibit an inflammatory response. In particular, Salp25C is useful to inhibit conditions characterized by the production of oxidative radical, for example, neutrophil and/or macrophage mediated inflammatory responses. Salp15 is useful to inhibit IL-2 and/or CD25 production, to treat conditions characterized by IL-2 and/or CD25 production, to inhibit T cell proliferation and to treat conditions characterized by a T cell response. Such conditions include but are not limited to autoimmune diseases, including lupus, arthritis and diabetes, and tissue and organ transplant rejection.

Salp25D appears to be a histamine-binding protein. Accordingly, Salp25 is useful to inhibit histamine activity and to treat conditions characterized by the production of histamine. Such conditions include, but are not limited to, hayfever, allergic reactions, respiratory infections, and other conditions that can be treated with anti-histamines, such as peptic ulcer disease.

Salp14A and Salp9A appear to be members of a family of I. scapularis proteins. Both have anti-coagulant activity and inhibit the activity of clotting factor Xa. Salp14A and Salp9A are useful in the treatment of blood coagulation related conditions including atherosclerosis, stroke, phlebitis, and, more generally, conditions that can be treated with heparin or coumadin.

It will be readily appreciated by one of ordinary skill in the art that the tick polypeptides and fragments of this invention, as well as fusion proteins and multimeric proteins containing them, may be prepared by recombinant means, chemical means, or combinations thereof.

For example, the tick polypeptides and fragments may be generated by recombinant means using a DNA sequence set forth in the sequence listing contained herein. DNA encoding serotypic variants of the polypeptides may likewise be cloned, e.g., using PCR and oligonucleotide primers derived from the sequence herein disclosed.

In this regard, it may be particularly desirable to isolate nucleic acid molecules encoding I. scapularis polypeptides from isolates that differ antigenically, i.e., Ixodes isolates against which I. scapularis polypeptides are ineffective to protect, in order to obtain a broad spectrum of different epitopes which would be useful in the methods and compositions of this invention.

Oligonucleotide primers and other nucleic-acid probes derived from the nucleic acid molecules encoding the polypeptides of this invention may also be used to isolate and clone other related proteins from I. scapularis and related ticks which may contain regions of DNA sequence homologous to the DNA sequences of this invention.

If the tick polypeptides and fragments of this invention are produced recombinantly, they may be expressed in unicellular hosts. As is well-known to one of skill in the art, in order to obtain high expression levels of foreign DNA sequences in a host, the sequences are generally operatively linked to transcriptional and translational expression control sequences that are functional in the chosen host. Preferably, the expression control sequences, and the DNA sequence of interest, will be contained in an expression vector that further comprises a selectable marker.

The nucleic acid sequences encoding the tick polypeptides and fragments of this invention may or may not encode a signal sequence. If the expression host is eukaryotic, it generally is preferred that a signal sequence be encoded so that the mature polypeptide is secreted from the eukaryotic host.

An amino-terminal methionine may or may not be present on the expressed tick polypeptides and fragments of this invention. If the terminal methionine is not cleaved by the expression host, it may, if desired, be chemically removed by standard techniques.

A wide variety of expression host/vector combinations may be employed in expressing the DNA sequences of this invention. Useful expression vectors for eukaryotic hosts, include, for example, vectors comprising expression control sequences from SV40, bovine papilloma virus, adenovirus, adeno-associated virus, cytomegalovirus and retroviruses including lentiviruses. Useful expression vectors for bacterial hosts include bacterial plasmids, such as those from E. coli, including pBluescript®, pGEX-2T, pUC vectors, colE1, pCR1, pBR322, pMB9 and their derivatives, pET-15, broad-host-range plasmids, such as RP4, phage DNAs, e.g., the numerous derivatives of phage lambda, e.g. λGT10 and λGT11, and other phages. Useful expression vectors for yeast cells include the 2μ plasmid and derivatives thereof. Useful vectors for insect cells include pVL941.

In addition, any of a wide variety of expression control sequences—sequences that control the expression of a DNA sequence when operatively linked to it—may be used in these vectors to express the DNA sequences of this invention. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the T3 and T7 promoters, the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast α-mating system and other constitutive and inducible promoter sequences known to control the expression of genes of prokaryotic or eukaryotic cells or their viruses, and various combinations thereof.

In a preferred embodiment, a DNA sequence encoding a tick polypeptide or fragment, preferably an I. scapularis polypeptide, of this invention is cloned in the expression vector lambda ZAP® II (Stratagene, La Jolla, Calif.), in which expression from the lac promoter may be induced by IPTG.

In another preferred embodiment, DNA encoding the I. scapularis polypeptides of this invention is inserted in-frame into an expression vector that allows high-level expression of the polypeptide or fragment as a glutathione S-transferase fusion protein. Such a fusion protein thus contains amino acids encoded by the vector sequences as well -as amino acids of the I. scapularis polypeptide.

In another preferred embodiment, DNA encoding the I. scapularis polypeptides of this invention is inserted in-frame into an expression vector that allows high-level expression of the polypeptide or fragment as thioredoxin fusion protein. Such a fusion protein thus contains amino acids encoded by the vector sequences as well as amino acids of the I. scapularis polypeptide.

The term “host cell” refers to one or more cells into which a recombinant DNA molecule is introduced. Host cells of the invention include, but need not be limited to, bacterial, yeast, animal, insect and plant cells. Host cells can be unicellular, or can be grown in tissue culture as liquid cultures, monolayers or the like. Host cells may also be derived directly or indirectly from tissues.

A wide variety of unicellular host cells are useful in expressing the DNA sequences of this invention. These hosts may include well-known eukaryotic and prokaryotic hosts, such as strains of E. coli, Pseudomonas, Bacillus, Streptomyces, fungi, yeast, insect cells such as Spodoptera frugiperda (SF9), animal cells such as CHO and mouse cells, African green monkey cells such as COS 1, COS 7, BSC 1, BSC 40, and BMT 10, and human cells, as well as plant cells.

A host cell is “transformed” by a nucleic acid when the nucleic acid is translocated into the cell from the extracellular environment. Any method of transferring a nucleic acid into the cell may be used; the term, unless otherwise indicated herein, do not imply any particular method of delivering a nucleic acid into a cell, nor that any particular cell type is the subject of transfer.

An “expression control sequence” is a nucleic-acid sequence that regulates gene expression (i.e., transcription, RNA formation and/or translation). Expression control sequences may vary depending, for example, on the chosen host cell or organism (e.g., between prokaryotic and eukaryotic hosts), the type of transcription unit (e.g., which RNA polymerase must recognize the sequences), the cell type in which the gene is normally expressed (and, in turn, the biological factors normally present in that cell type).

A “promoter” is one such expression control sequence, and, as used herein, refers to an array of nucleic-acid sequences which control, regulate and/or direct transcription of downstream (3′) nucleic-acid sequences. As used herein, a promoter includes necessary nucleic-acid sequences near the start site of transcription, such as, in the case of a polymerase II type promoter, a TATA element.

A “constitutive” promoter is a promoter which is active under most environmental and developmental conditions. An “inducible” promoter is a promoter which is inactive under at least one environmental or developmental condition and which can be switched “on” by altering that condition. A “tissue-specific” promoter is active in certain tissue types of an organism, but not in other tissue types from the same organism. Similarly, a developmentally regulated promoter is active during some but not all developmental stages of a host organism.

Expression control sequences also include distal enhancer or repressor elements which can be located as much as several thousand base pairs from the start site of transcription. They also include sequences required for RNA formation (e.g., capping, splicing, 3′ end formation and poly-adenylation, where appropriate); translation (e.g., ribosome binding site); and post-translational modifications (e.g., glycosylation, phosphorylation, methylation, prenylation, and the like).

The term “operatively linked” refers to functional linkage between a nucleic-acid expression control sequence (such as a promoter, or array of transcription factor binding sites) and a second nucleic-acid sequence, wherein the expression control sequence directs transcription of the nucleic acid corresponding to the second sequence.

The term “polypeptide” refers to any polymer consisting essentially of amino acids regardless of its size. Although “protein” is often used in reference to relatively large polypeptides, and “peptide” is often used in reference to small polypeptides, usage of these terms in the art overlaps and varies. The term “polypeptide” as used herein thus refers interchangeably to peptides, polypeptides and proteins, unless otherwise noted.

The term “amino acid” refers to a monomeric unit of a peptide, polypeptide or protein.

It should of course be understood that not all vectors and expression control sequences will function equally well to express the DNA sequences of this invention. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must be replicated in it. The vector's copy number, the ability to control that copy number, the ability to control integration, if any, and the expression of any other proteins encoded by the vector, such as antibiotic or other selectable markers, should also be considered.

In selecting an expression control sequence, a variety of factors should also be considered. These include, for example, the relative strength of the promoter sequence, its controllability, and its compatibility with the DNA sequence of this invention, particularly with regard to potential secondary structures. Unicellular hosts should be selected by consideration of their compatibility with the chosen vector, the toxicity of the product coded for by the DNA sequences of this invention, their secretion characteristics, their ability to fold the polypeptide correctly, their fermentation or culture requirements, and the ease of purification from them of the products coded for by the DNA sequences of this invention.

Within these parameters, one of skill in the art may select various vector/expression control sequence/host combinations that will express the DNA sequences of this invention on fermentation or in other large-scale cultures.

The polypeptides encoded by the nucleic acid molecules of this invention may be isolated from the fermentation or cell culture and purified using any of a variety of conventional methods including: liquid chromatography such as normal or reversed-phase, using HPLC, FPLC and the like; affinity chromatography (such as with inorganic ligands or monoclonal antibodies); size-exclusion chromatography; immobilized metal chelate chromatography; gel electrophoresis; and the like. One of skill in the art may select the most appropriate isolation and purification techniques without departing from the scope of this invention. If the polypeptide is membrane-bound or suspected of being a lipoprotein, it may be isolated using methods known in the art for such proteins, e.g., using any of a variety of suitable detergents.

In addition, the polypeptides and fragments of the invention may be generated by any of several chemical techniques. For example, they may be prepared using the solid-phase synthetic technique originally described by R. B. Merrifield, “Solid Phase Peptide Synthesis. I. The Synthesis of a Tetrapeptide”, J. Am. Chem. Soc., 83, pp. 2149-54 (1963), or they may be prepared by synthesis in solution. A summary of peptide synthesis techniques may be found in E. Gross & H. J. Meinhofer, 4. The Peptides: Analysis, Synthesis, Biology; Modern Techniques of Peptide and Amino Acid Analysis, John Wiley & Sons, (1981) and M. Bodanszky, Principles of Peptide Synthesis, Springer-Verlag (1984).

Typically, these synthetic methods comprise the sequential addition of one or more amino-acid residues to a growing peptide chain. Often peptide coupling agents are used to facilitate this reaction. For a recitation of peptide coupling agents suitable for the uses described herein see M. Bodansky, supra. Normally, either the amino or carboxyl group of the first amino-acid residue is protected by a suitable, selectively removable protecting group. A different protecting group is utilized for amino acids containing a reactive side group, e.g., lysine. A variety of protecting groups known in the field of peptide synthesis and recognized by conventional abbreviations therein, may be found in T. Greene, Protective Groups In Organic Synthesis, Academic Press (1981).

According to another embodiment of this invention, antibodies that specifically bind a tick polypeptide of the invention, preferably an I. scapularis polypeptide, are generated. Such antibodies are immunoglobulin molecules or portions thereof that are immunologically reactive with a polypeptide of the present invention. It should be understood that the antibodies of this invention include antibodies immunologically reactive with fusion proteins and multimeric proteins comprising an I. scapularis polypeptide.

Antibodies directed against an I. scapularis polypeptide may be generated by a variety of means including immunizing a mammalian host with I. scapularis extract or by tick infestation, or by immunization of a mammalian host with an I. scapularis polypeptide of the present invention or immunogenic fragment thereof. Such antibodies may be polyclonal or monoclonal. It is preferred that they are monoclonal. Methods to produce polyclonal and monoclonal antibodies are well-known to those of skill in the art. For a review of such methods, see Antibodies, A Laboratory Manual, supra, and D. E. Yelton, et al., “Monoclonal Antibodies: A Powerful New Tool in Biology and Medicine,” Ann. Rev. of Biochem., 50, pp. 657-80 (1981). Determination of immunoreactivity with an I. scapularis polypeptide of this invention may be made by any of several methods well-known in the art, including by immunoblot assay and ELISA.

An antibody of this invention may also be a hybrid molecule formed from immunoglobulin sequences from different species (e.g., mouse and human) or from portions of immunoglobulin light- and heavy-chain sequences from the same species. It may be a molecule that has multiple binding specificities, such as a bifunctional antibody prepared by any one of a number of techniques known to those of skill in the art including: the production of hybrid hybridomas; disulfide exchange; chemical cross-linking; addition of peptide linkers between two monoclonal antibodies; the introduction of two sets of immunoglobulin heavy and light chains into a particular cell line; and so forth.

The antibodies of this invention may also be human monoclonal antibodies produced by any of the several methods known in the art. For example, human monoclonal antibodies may be produced by immortalized human cells, by SCID-hu mice, by the expression of cloned human immunoglobulin genes, by phage-display, or by any other method known in the art. A human antibody or an antigen-binding portion thereof of the invention also can be produced in non-human animals capable of producing human antibodies. See e.g., International Patent publication WO 98/24893.

In addition, it may be advantageous to couple the antibodies of this invention to toxins such as diphtheria, pseudomonas exotoxin, ricin A chain, gelonin, etc., or antibiotics such as penicillins, tetracyclines and chloramphenicol.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

One of skill in the art will understand that antibodies directed against a tick polypeptide of the invention may have utility in prophylactic compositions and methods directed against tick bite and infection with a tick-borne pathogen. For example, the level of pathogens in infected ticks may be decreased by allowing them to feed on the blood of animals immunized with a polypeptide of this invention.

The antibodies of this invention also have a variety of other uses. For example, they are useful as reagents to screen for expression of the I. scapularis polypeptides, either in libraries constructed from I. scapularis nucleic acid molecules or from other samples in which the proteins may be present. Moreover, by virtue of their specific binding affinities, the antibodies of this invention are also useful to purify or remove polypeptides from a given sample, to block or bind to specific epitopes on the polypeptides and to direct various molecules, such as toxins, to ticks.

To screen the I. scapularis polypeptides and antibodies of this invention for their ability to confer protection against tick bite or their ability to lessen the severity of infection with tick-borne pathogens, guinea pigs are preferred as an animal model. Of course, while any animal that can acquire tick immunity may be useful, guinea pigs are not only a classical model for tick immunity but also display skin reactivity that mimics hypersensitivity reactions in humans. Thus, by administering a particular I. scapularis polypeptide or anti-I. scapularis polypeptide antibody to guinea pigs, one of skill in the art may determine without undue experimentation whether that polypeptide or antibody would be useful in the methods and compositions claimed herein.

The administration of the I. scapularis polypeptide or antibody of this invention to the animal may be accomplished by any of the methods disclosed herein or by a variety of other standard procedures. For a detailed discussion of such techniques, see Antibodies, A Laboratory Manual, supra. Preferably, if a polypeptide is used, it will be administered with a pharmaceutically acceptable adjuvant, such as complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

Once the I. scapularis polypeptides or antibodies of this invention have been determined to be effective in the screening process, they may then be used in a therapeutically effective amount in pharmaceutical compositions and methods to confer tick immunity and to prevent or reduce the transmission of tick-borne pathogens.

The pharmaceutical compositions of this invention may be in a variety of conventional depot forms. These include, for example, solid, semi-solid and liquid dosage forms, such as tablets, pills, powders, liquid solutions or suspensions, liposomes, capsules, suppositories, injectable and infusible solutions. The preferred form depends upon the intended mode of administration and prophylactic application.

Such dosage forms may include pharmaceutically acceptable carriers and adjuvants which are known to those of skill in the art. These carriers and adjuvants include, for example, RIBI, ISCOM, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes such as protamine sulfate, disodium hydrogen phosphate, sodium chloride, zinc salts, colloidal silica, magnesium trisilicate, polyvinyl pyrrolidone, cellulose-based substances, and polyethylene glycol. Adjuvants for topical or gel-base forms may be selected from the group consisting of sodium carboxymethylcellulose, polyacrylates, polyoxyethylene-polyoxypropylene-block polymers, polyethylene glycol, and wood wax alcohols.

The vaccines and compositions of this invention may also include other components or be subject to other treatments during preparation to enhance their immunogenic character or to improve their tolerance in patients.

Compositions comprising an antibody of this invention may be administered by a variety of dosage forms and regimens similar to those used for other passive immunotherapies and well-known to those of skill in the art. Generally, the I. scapularis polypeptides may be formulated and administered to the patient using methods and compositions similar to those employed for other pharmaceutically important polypeptides (e.g., the vaccine against hepatitis).

Any pharmaceutically acceptable dosage route, including parenteral, intravenous, intramuscular, intralesional or subcutaneous injection, may be used to administer the polypeptide or antibody composition. For example, the composition may be administered to the patient in any pharmaceutically acceptable dosage form including those which may be administered to a patient intravenously as bolus or by continued infusion over a period of hours, days, weeks or months, intramuscularly—including paravertebrally and periarticularly—subcutaneously, intracutaneously, intra-articularly, intrasynovially, intrathecally, intralesionally, periostally or by oral or topical routes. Preferably, the compositions of the invention are in the form of a unit dose and will usually be administered to the patient intramuscularly.

The I. scapularis polypeptides or antibodies of this invention may be administered to the patient at one time or over a series of treatments. The most effective mode of administration and dosage regimen will depend upon the level of immunogenicity, the particular composition and/or adjuvant used for treatment, the severity and course of the expected infection, previous therapy, the patient's health status and response to immunization, and the judgment of the treating physician.

For example, in an immunocompetent patient, the more highly immunogenic the polypeptide, the lower the dosage and necessary number of immunizations. Similarly, the dosage and necessary treatment time will be lowered if the polypeptide is administered with an adjuvant. Generally, the dosage will consist of 10 μg to 100 mg of the purified polypeptide, and preferably, the dosage will consist of 10-1000 μg. Generally, the dosage for an antibody will be 0.5 mg-3.0 g.

In a preferred embodiment of this invention, the I. scapularis polypeptide is administered with an adjuvant, in order to increase its immunogenicity. Useful adjuvants include RIBI, and ISCOM, simple metal salts such as aluminum hydroxide, and oil-based adjuvants such as complete and incomplete Freund's adjuvant. When an oil-based adjuvant is used, the polypeptide usually is administered in an emulsion with the adjuvant.

In yet another preferred embodiment, E. coli expressing proteins comprising an I. scapularis polypeptide are administered orally to non-human animals according to methods known in the art, to confer tick immunity and to prevent or reduce the transmission of tick-borne pathogens. For example, a palatable regimen of bacteria expressing an I. scapularis polypeptide, alone or in the form of a fusion protein or multimeric protein, may be administered with animal food to be consumed by wild mice or other animals that act as alternative hosts for I. scapularis ticks.

Ingestion of such bacteria may induce an immune response comprising both humoral and cell-mediated components. See J. C. Sadoff et al., “Oral Salmonella typhimurium Vaccine Expressing Circumsporozoite Protein Protects Against Malaria”, Science, 240, pp. 336-38 (1988) and K. S. Kim et al., “Immunization of Chickens with Live Escherichia coli Expressing Eimeria acervulina Merozoite Recombinant Antigen Induces Partial Protection Against Coccidiosis”, Infect. Immun., 57, pp. 2434-40 (1989); M. Dunne et al., “Oral Vaccination with an Attenuated Salmonella typhimurium Strain Expressing Borrelia burgdorferi OspA Prevents Murine Lyme Borreliosis,” Infect. Immun., 63:1611-14 (1995); E. Fikrig et al., “Protection of Mice from Lyme Borreliosis by Oral Vaccination with Escherichia coli Expressing OspA,” J. Infect. Dis., 164:1224-27 (1991).

Moreover, the level of pathogens in ticks feeding on such animals may be lessened or eliminated, thus inhibiting transmission to the next animal.

According to yet another embodiment, the tick polypeptides, preferably I. scapularis polypeptides, of this invention, and the nucleic acid molecules encoding them are useful as diagnostic agents for detecting tick immunity and tick bite. The polypeptides are capable of binding to antibody molecules produced in animals, including humans, that have been exposed to I. scapularis antigens as a result of a tick bite. The detection of I. scapularis antigens is evidence of tick attachment and at least some feeding. Such information is an important aid in the early diagnosis of I. scapularis-borne diseases.

Such diagnostic agents may be included in a kit which may also comprise instructions for use and other appropriate reagents, preferably a means for detecting when the polypeptide or antibody is bound. For example, the polypeptide or antibody may be labeled with a detection means that allows for the detection of the polypeptide when it is bound to an antibody, or for the detection of the antibody when it is bound to I. scapularis or an antigen thereof.

The detection means may be a fluorescent labeling agent such as fluorescein isocyanate (FIC), fluorescein isothiocyanate (FITC), and the like, an enzyme, such as horseradish peroxidase (HRP), glucose oxidase or the like, a radioactive element such as ¹²⁵I or ⁵¹Cr that produces gamma-ray emissions, or a radioactive element that emits positrons which produce gamma rays upon encounters with electrons present in the test solution, such as ¹¹C, ¹⁵O, or ¹³N. Binding may also be detected by other methods, for example via avidin-biotin complexes.

The linking of the detection means is well-known in the art. For instance, monoclonal antibody molecules produced by a hybridoma can be metabolically labeled by incorporation of radioisotope-containing amino acids in the culture medium, or polypeptides may be conjugated or coupled to a detection means through activated functional groups.

The diagnostic kits of the present invention may be used to detect the presence of anti-I. scapularis antibodies in a body fluid sample such as serum, plasma or urine. Thus, in preferred embodiments, an I. scapularis polypeptide or an antibody of the present invention is bound to a solid support typically by adsorption from an aqueous medium. Useful solid matrices are well-known in the art, and include cross-linked dextran; agarose; polystyrene; polyvinylchloride; cross-linked polyacrylamide; nitrocellulose or nylon-based materials; tubes, plates or the wells of microtiter plates. The polypeptides or antibodies of the present invention may be used as diagnostic agents in solution form or as a substantially dry powder, e.g., in lyophilized form.

I. scapularis polypeptides and antibodies directed against those polypeptides provide much more specific diagnostic reagents than whole ticks preparations and thus may alleviate such pitfalls as false-positive and false-negative results.

One skilled in the art will realize that it may also be advantageous in the preparation of detection reagents to utilize epitopes from more than one I. scapularis polypeptide and antibodies directed against such epitopes.

The skilled artisan also will realize that it may be advantageous to prepare a diagnostic kit comprising diagnostic reagents to detect I. scapularis polypeptides or antibodies as well as polypeptides or antibodies from pathogens found in the same tick vector, for example, Borrelia burgdorferi, Babesia microti, aoHGE (the agent of human granulocytic ehrlichiosis), and some arboviruses, such as the Eastern equine encephalitis virus.

The polypeptides and antibodies of the present invention, and compositions and methods comprising them, may also be useful to prevent tick bites by ticks other that I. scapularis. Such ticks may express polypeptides that share amino-acid sequence or conformational similarities with the I. scapularis polypeptides of the present invention.

Throughout this specification and claims, the word “comprise,” or variations such as “comprises” or “comprising,” will be understood to imply the inclusion of a stated integer or group of integers but not the exclusion of any other integer or group of integers.

In order that this invention may be better understood, the following examples are set forth. These examples are for purposes of illustration only, and are not to be construed as limiting the scope of the invention in any manner.

### EXAMPLE I

### Cloning Nucleic Acid Molecules Encoding I. scapularis Salivary Gland Proteins

A. Preparing cDNA Libraries

To obtain I. scapularis salivary glands for preparation of a cDNA expression library, over a 4-week period, we fed 1,000 I. scapularis nymphs on naive 5-6 week old C3H/HeJ mice. After 72 hours, we pulled off the ticks and kept them under humidified conditions until dissection, which was within 24 hours of being pulled.

For dissection, we placed the ticks over a drop of PBS on a cover slip and cut them in half using a spear and sharp-pointed tweezers. We transferred the upper half of the body to a second drop of PBS within the cover slip and cut lengthwise. We scooped the interior contents of the upper segment from the shell and recovered the pair of salivary glands. We kept the salivary glands under guanidium/β-mercaptoethanol until all dissections were complete to prevent degradation by RNases.

We isolated RNA using Stratagene's Micro RNA Isolation Kit. Briefly, we added 30 μl of 2M sodium acetate, 300 μl of water-saturated phenol and 60 μl of chloroform:isoamyl alcohol to a 300 μl aliquot of salivary gland in CITC/β-mercaptoethanol. We capped the tube, vortexed and microfuged for 5 min at maximum speed.

We transferred the upper phase containing the RNA to a new tube, added glycogen carrier and isopropanol and microfuged for 30 min in the cold to precipitate RNA. We washed the pellet in 75% ethanol and dried in a vacuum for 5 min. We resuspended the RNA in water and read an aliquot in a spectrophotometer at 260 nm. Our yield was 0.1-0.27 μg total RNA per tick. We sent the isolated RNA to Clontech Laboratories, Inc. where a Lambda ZAP® II expression library was made after initial amplification of the message.

B. Screening Ixodes Libraries With Hyperimmune and Immune Sera

To identify antigens recognized by tick-immune sera, we screened the cDNA libraries as follows.

We prepared whole-tick immune sera by infesting rabbits with 10 adult I. scapularis ticks 3 times at 21-day intervals.

We sacrificed the animals 15 days after the final tick feeding and collected blood by heart puncture. We left the blood at room temperature for 2 hours for clot formation, then isolated the immune sera by centrifugation at 1000×g for 5 min. We stored the sera at −20° C. until further use.

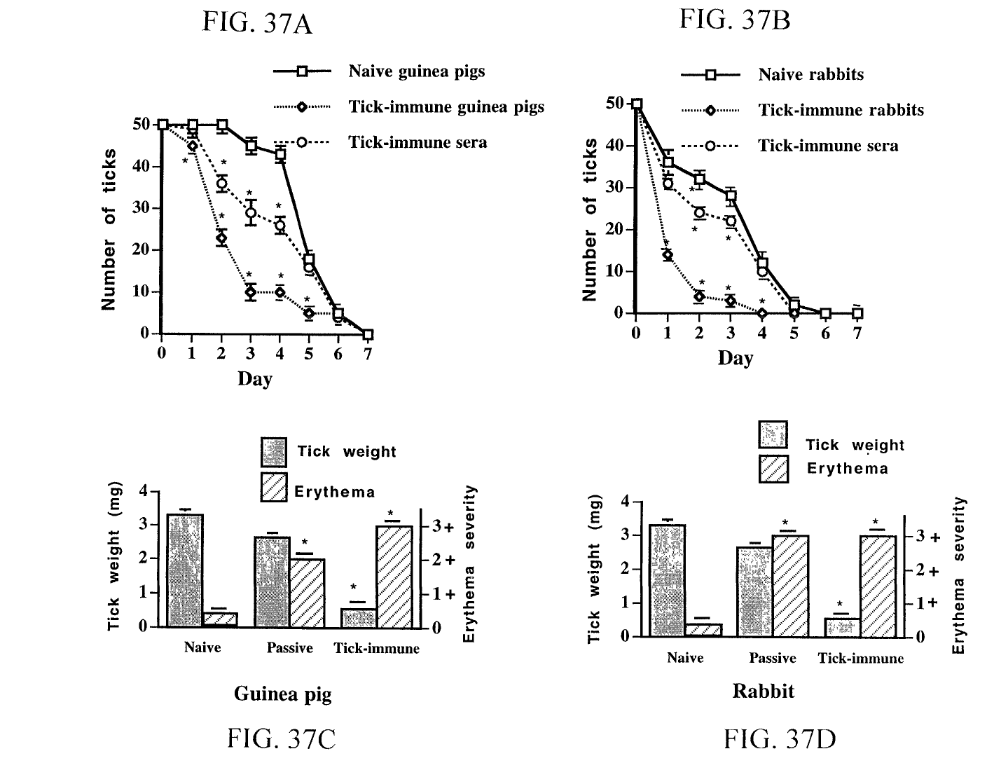

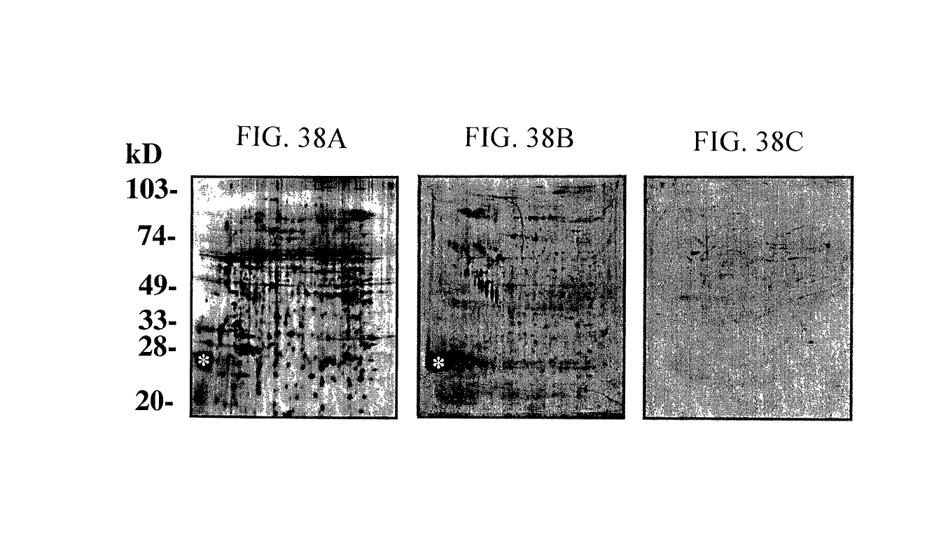

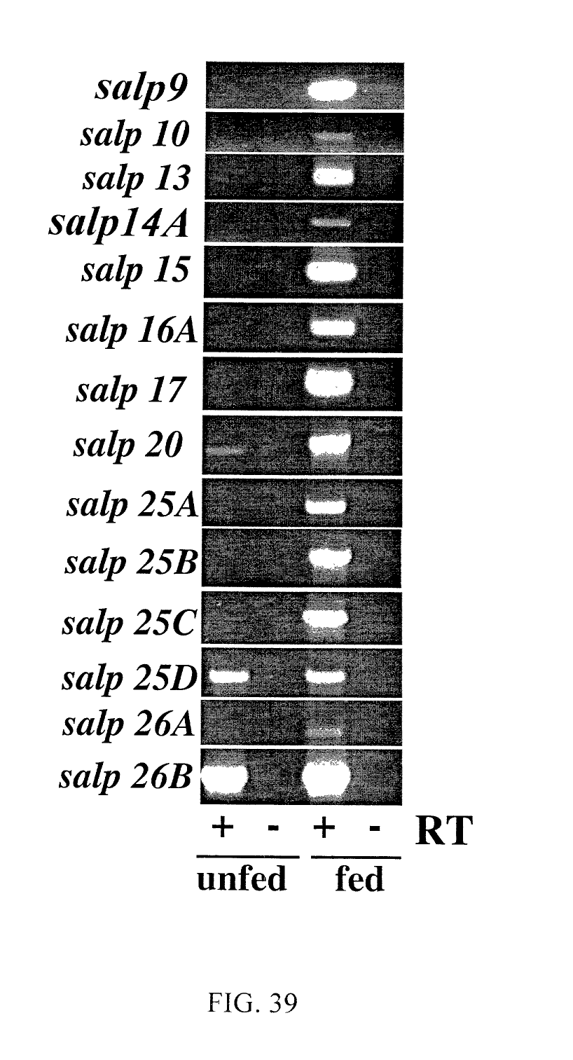

To confirm that these animals had developed tick-immunity, we challenged groups of 3 tick-sensitized animals with 50 I. scapularis nymphs and monitored the progress of the tick infestation. We recorded the duration of tick attachment, the weight of the recovered ticks and the appearance of erythema at the bite sites. We observed that ticks did not readily attach and engorge on tick-sensitized animals compared with naive (control) animals (FIGS. 37A-D). Furthermore, we observed erythema at the tick-bite sites of tick-sensitized, but not control, animals (FIGS. 37A-D).

We grew approximately 100,000 Lambda phage plaques containing the I. scapularis salivary gland cDNA (prepared as in part A) on E. coli XL1-Blue cell lawns in 90 mm culture plates. We then induced expression of the cDNA with 10 mM IPTG in a soaked nitrocellulose membrane for 3 hours and probed the membranes with tick-immune rabbit sera in 2-10 fold dilutions. As controls, we probed replica plates with normal rabbit sera.

After washing, we incubated the filters with alkaline phosphate conjugated goat anti-rabbit antibody to detect clones. The tick-immune sera recognized 47 different clones from the salivary gland cDNA library. We plated individual plaques for secondary and tertiary screening with immune sera and purified to homogeneity.

We excised the inserts from the clones using the R408 helper phage and digested the vectors with the inserts with EcoR1 endonuclease.

To identify additional I. scapularis antigens, we rescreen the expression libraries with immune sera from other mammals, for example mice and humans, according to the methods described herein.

C. Sequencing the Inserts

The inserts of the clones were sequenced by the Sanger method in the HHMI Biopolymer/Keck Foundation Biotechnology Resource Laboratory, Yale University School of Medicine, 333 Cedar Street, New Haven, Conn.

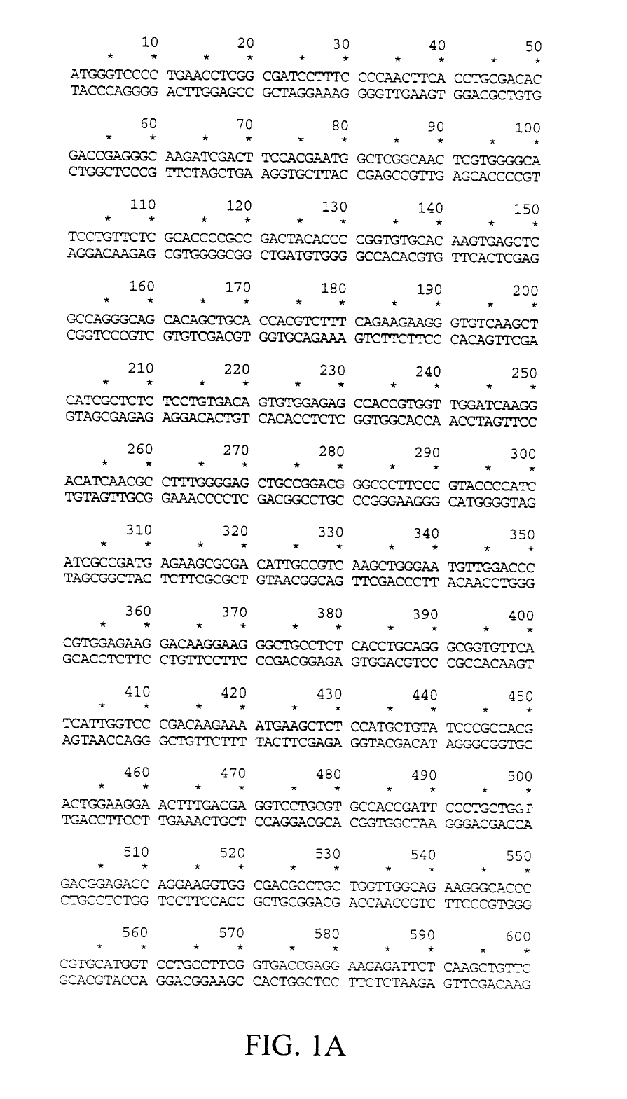

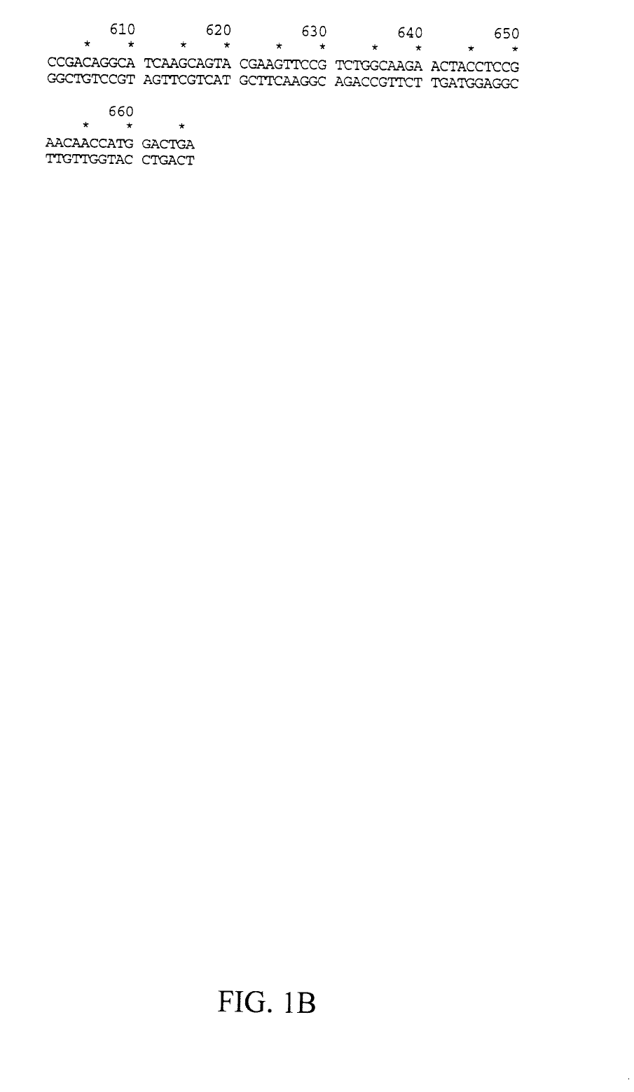

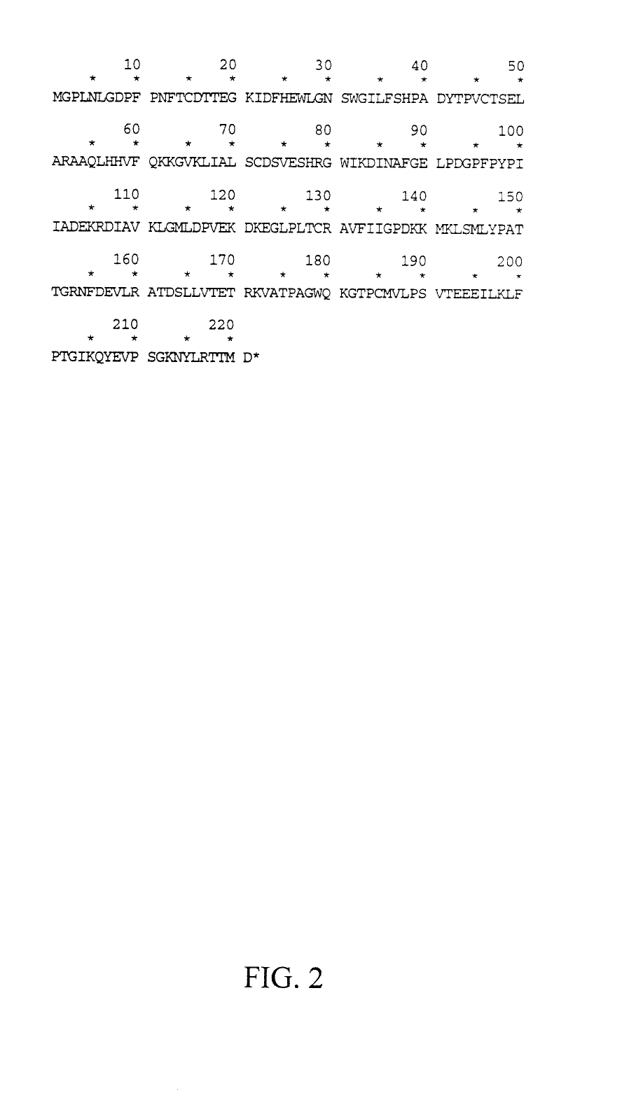

Analysis of the inserts in the forty-seven clones by sequence and cross-hybridization on Southern blot revealed that we had isolated fifteen complete reading frames encoding fifteen novel immunodominant tick antigens. Two clones each contained an 666 bp open reading frame that encoded a 221-amino-acid polypeptide with an expected molecular weight of 25 kDa. We designated one of the genes as salp25C (FIGS. 1A-1B and SEQ ID NO: 1) and the protein encoded by the gene as Salp25C. The deduced amino-acid sequence of the Salp25C polypeptide is set forth in FIG. 2 and SEQ ID NO: 2. This protein has a predicted molecular weight of 24.6 kDa and a pI of 5.7. We performed a BLAST search of the Genbank database which revealed homology with vertebrate and invertebrate glutathione peroxidases.

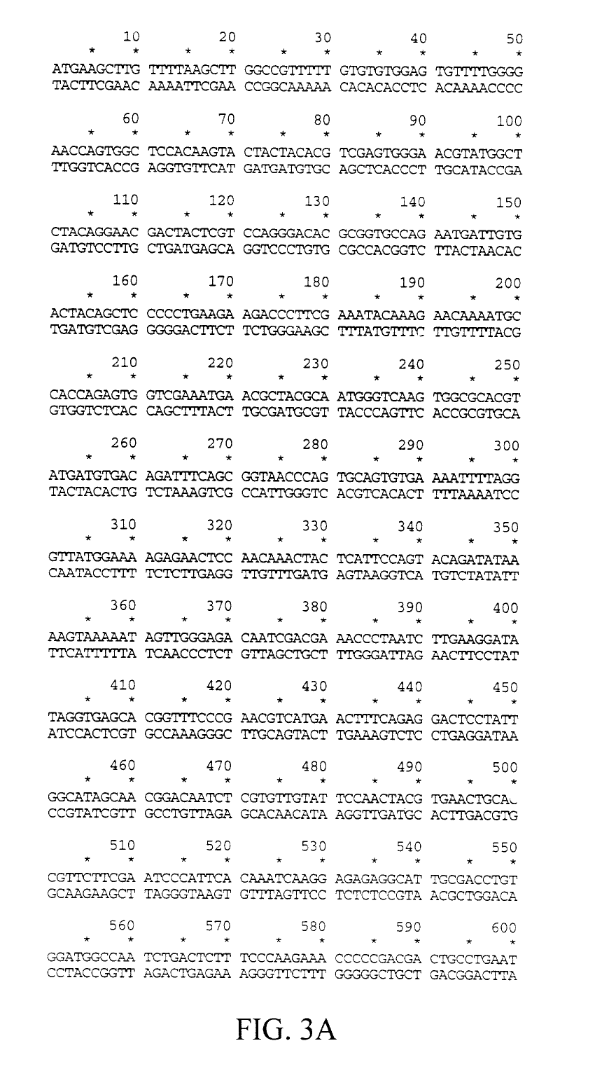

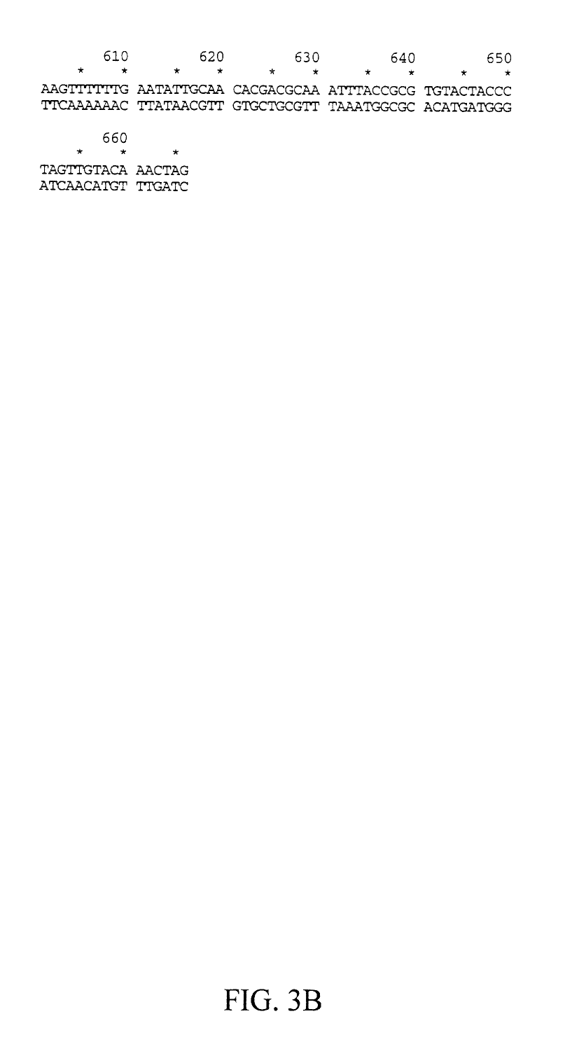

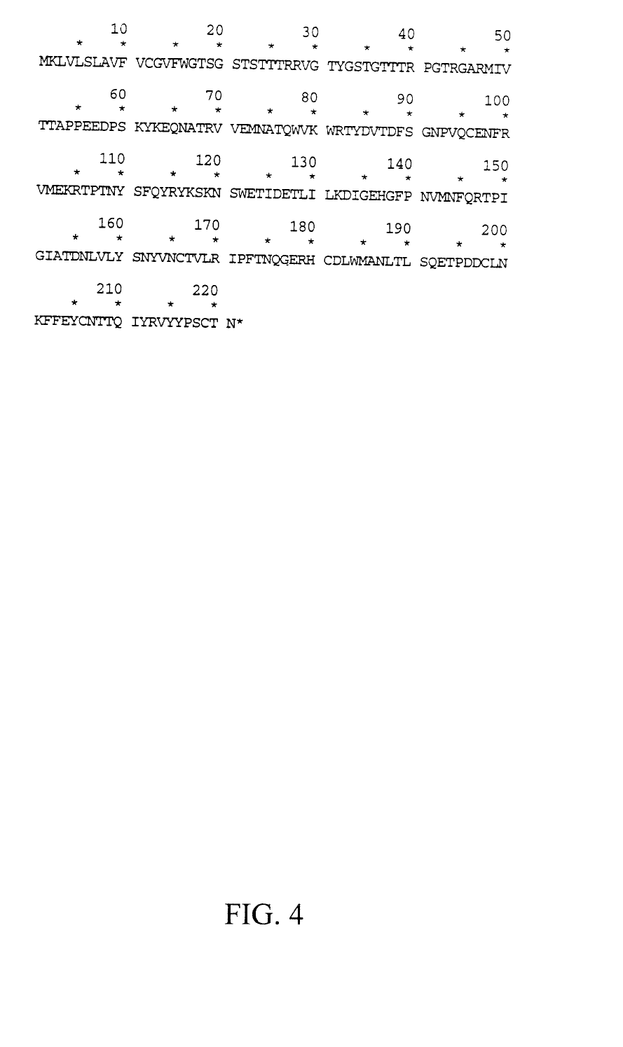

We designated the other clone as salp25D (FIGS. 3A-3B and SEQ ID NO: 3) and the protein encoded by the gene as Salp25D. The deduced amino-acid sequence of the Salp25D polypeptide is set forth in FIG. 4 and SEQ ID NO: 4. This protein has a predicted molecular weight of 25.3 kDa, a pI of 8.7 and a 21-amino-acid signal sequence. This protein has homology with the histamine-binding protein from Riphicephalus ticks [Paesen et al., “Tick Histamine-binding Proteins: Isolation, Cloning, and Three-dimensional Structure,” Mol. Cell, 3, pp. 661-671 (1999)]. We performed ProfileScan on the Prosite database which confirmed that the protein possesses histamine-binding motifs.

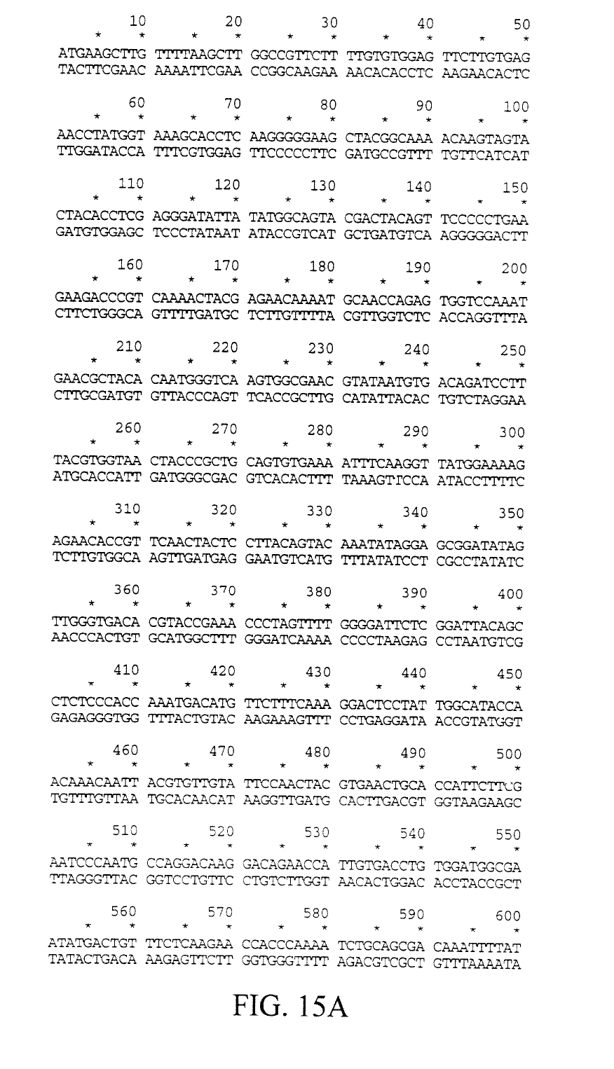

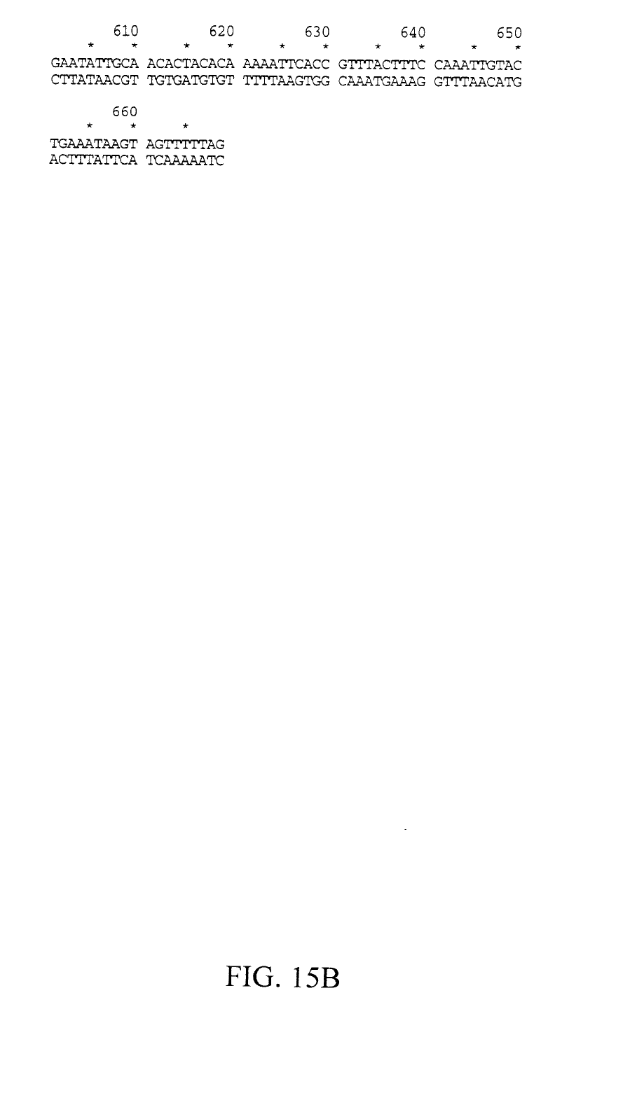

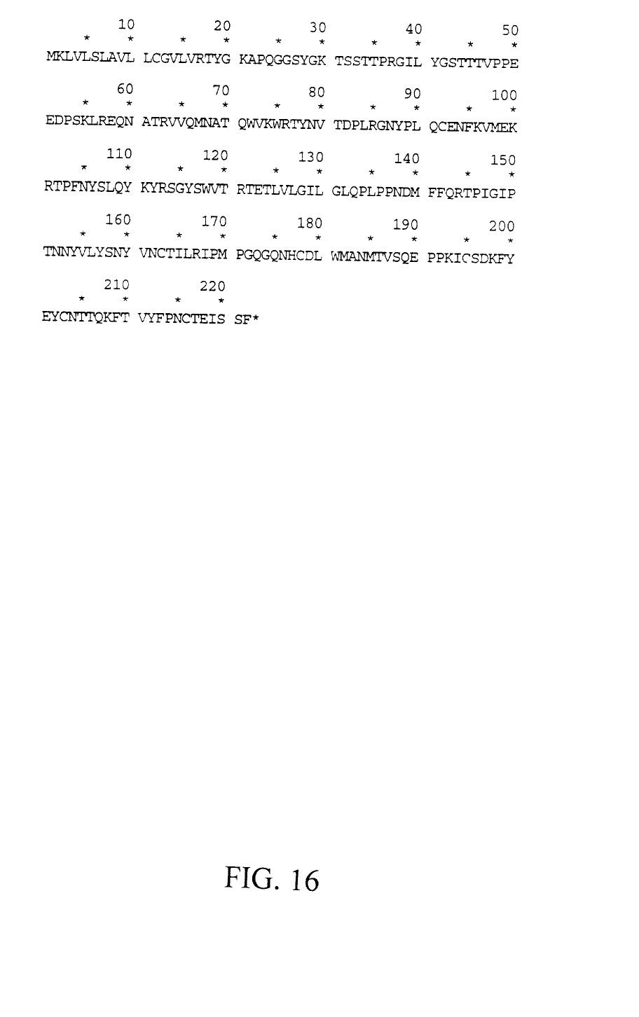

Another two clones each contained a 669 bp open reading frame that encoded a 222-amino-acid polypeptide with an expected molecular weight of 25 kDa. We designated one of the genes as salp25A (FIGS. 15A-15B and SEQ ID NO: 15) and the protein encoded by the gene as Salp25A. The deduced amino-acid sequence of the Salp25A polypeptide is set forth in FIG. 16 and SEQ ID NO: 16. This protein has a predicted molecular weight of 25.4 kDa, a pI of 9.5 and a 20-amino-acid signal sequence.

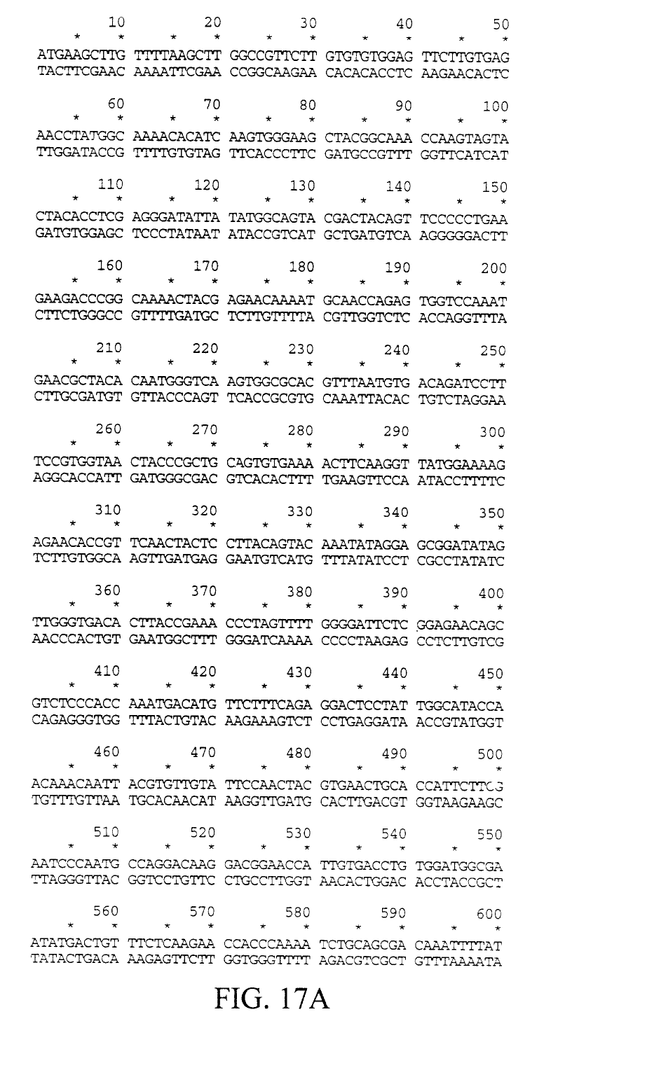

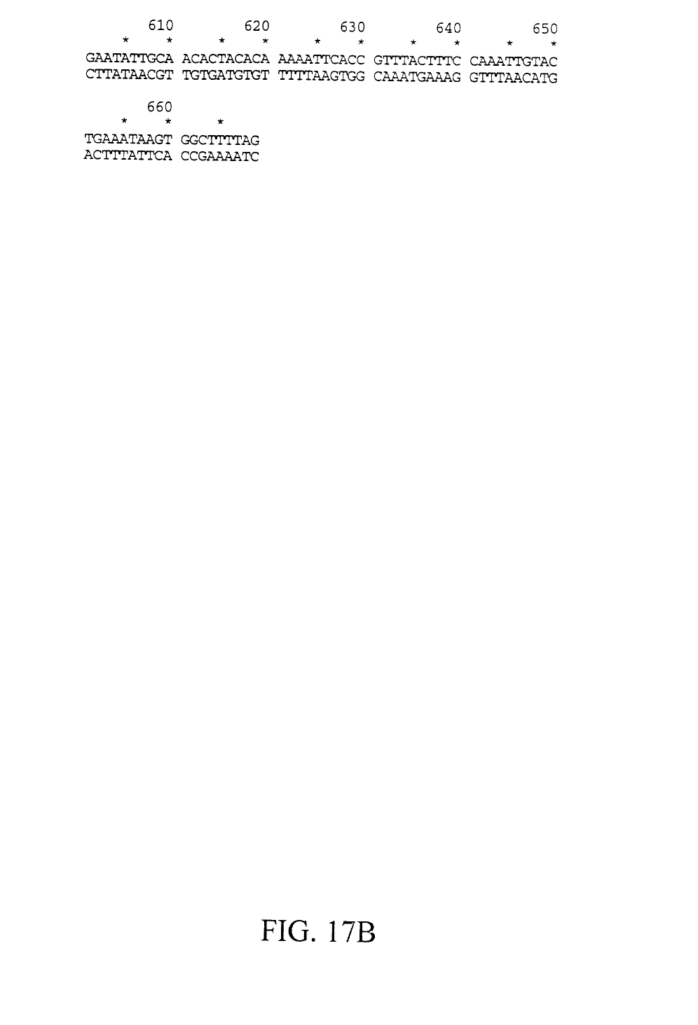

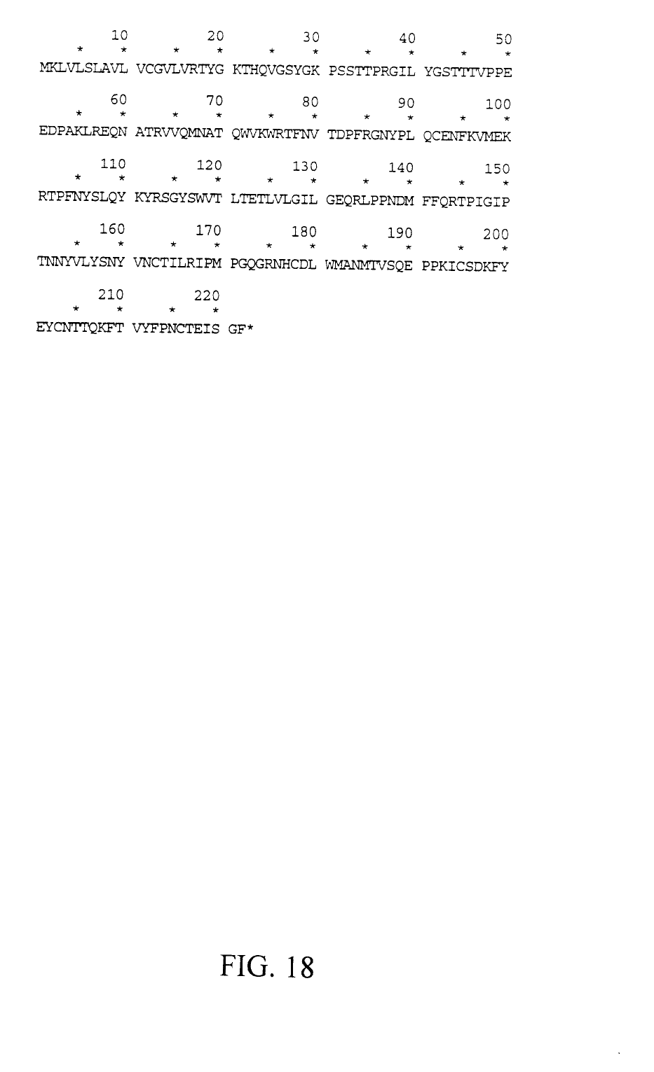

We designated the other clone as salp25B (FIGS. 17A-17B and SEQ ID NO: 17) and the protein encoded by the gene as Salp25B. The deduced amino-acid sequence of the Salp25B polypeptide is set forth in FIG. 18 and SEQ ID NO: 18. This protein has a predicted molecular weight of 25.5 kDa, a pI of 9.6 and a 21-amino-acid signal sequence.

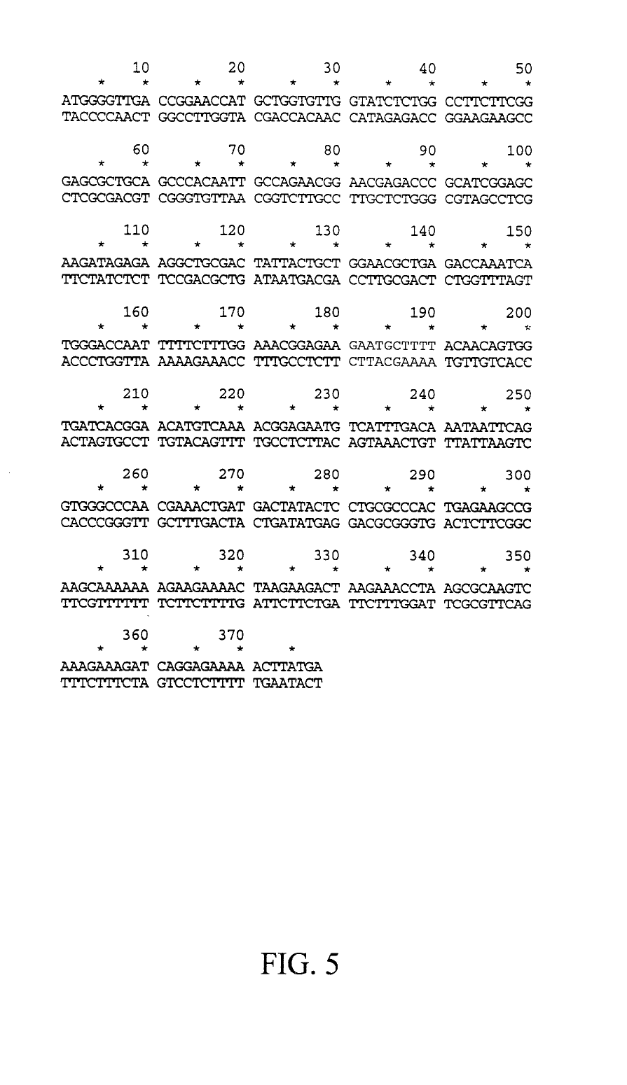

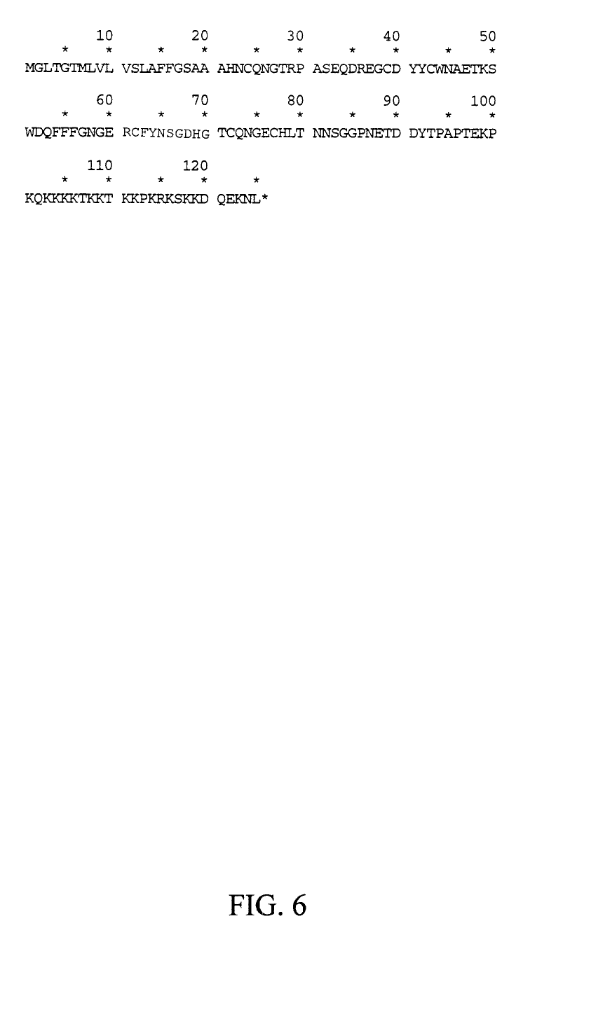

A fifth clone contained a 378 bp open reading frame that encodes a 125-amino-acid polypeptide with an expected molecular weight of 14.0 kDa, a pI of 9.2 and a 21-amino-acid signal sequence. We designated the gene as salp14A (FIG. 5 and SEQ ID NO: 5) and the protein encoded by the gene as Salp14A. The deduced amino-acid sequence of the Salp14A polypeptide is set forth in FIG. 6 and SEQ ID NO: 6. Salp14A appears to be one member of a family of proteins which also includes the Salp9A protein. The DNA sequence encoding the Salp9A protein is: AGACCGGCCACTGAGGAGAAGAGAGAAGGCTGCGACTATTACTGCTGGAAC ACCGAGACCAAATCATGGGACAAATTTTTCTTCGGGAACGGAGAACGATGC TTTTACAACAATGGTGATGAGGGATTATGTCAAAACGGAGAGTGCCATTTG ACAACAGATTCAGGTATGCCCAATGACACTGATGCAAAAATAGAAGAAACC GAAGAAGAGTTAGAAGCCTAA (SEQ ID NO: 35) and the deduced amino-acid sequence of the Salp9A protein is: RPATEEKREGCDYYCWN TETKSWDKFFFGNGERC FYNNGDEGLCQNGECHL TTDSGMPNDTDAKIEET EEELEA* (SEQ ID NO: 36).

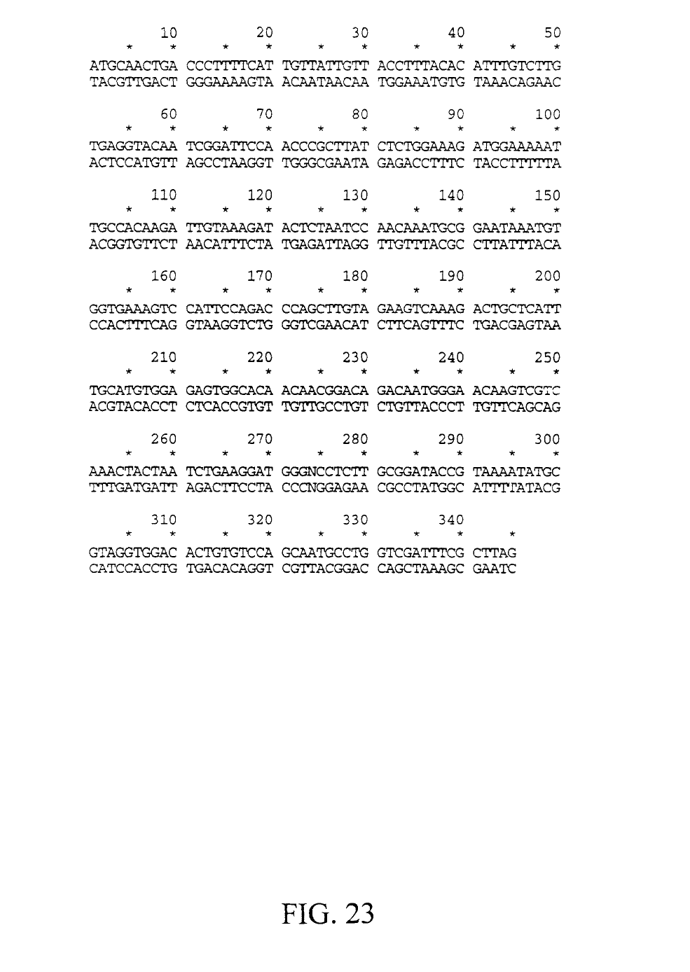

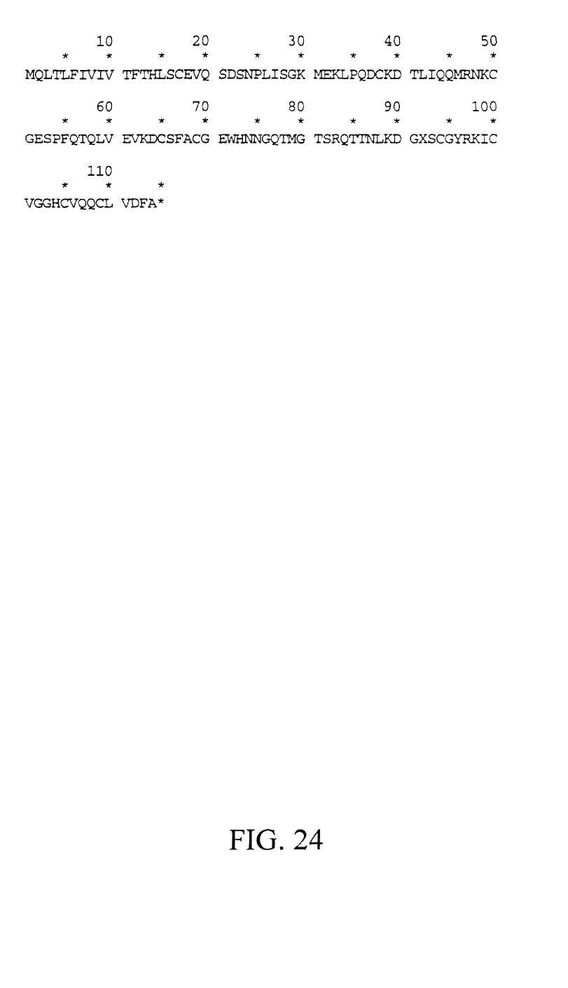

A sixth clone contained a 345 bp open reading frame that encodes a 114-amino-acid polypeptide with an expected molecular weight of 14 kDa. We designated the gene as salp14B (FIG. 23 and SEQ ID NO: 23) and the protein encoded by the gene as Salp14B. The deduced amino-acid sequence of the Salp14B polypeptide is set forth in FIG. 24 and SEQ ID NO: 24.

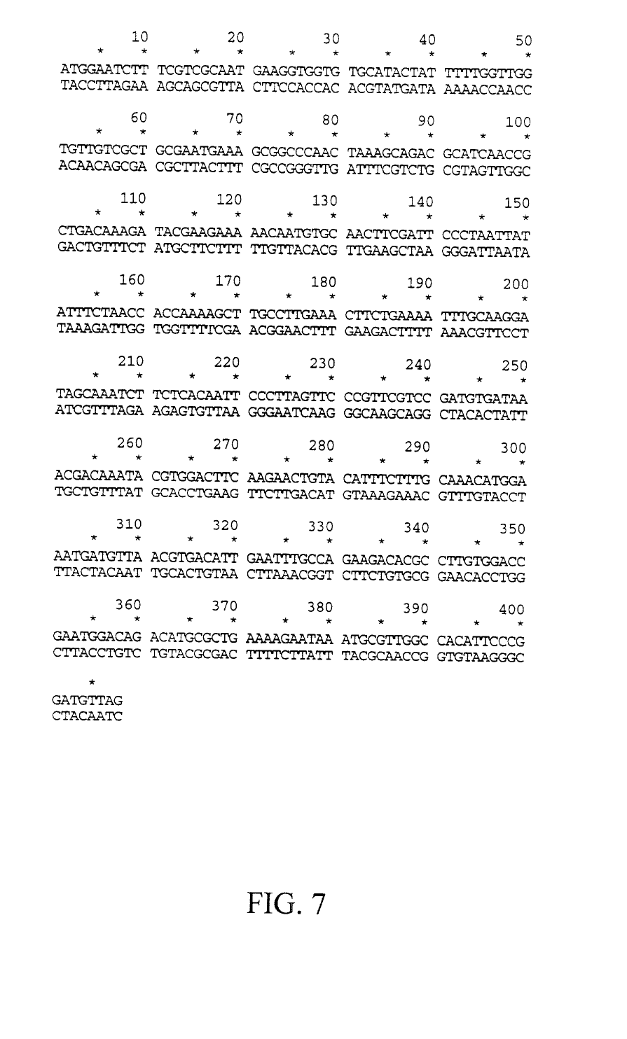

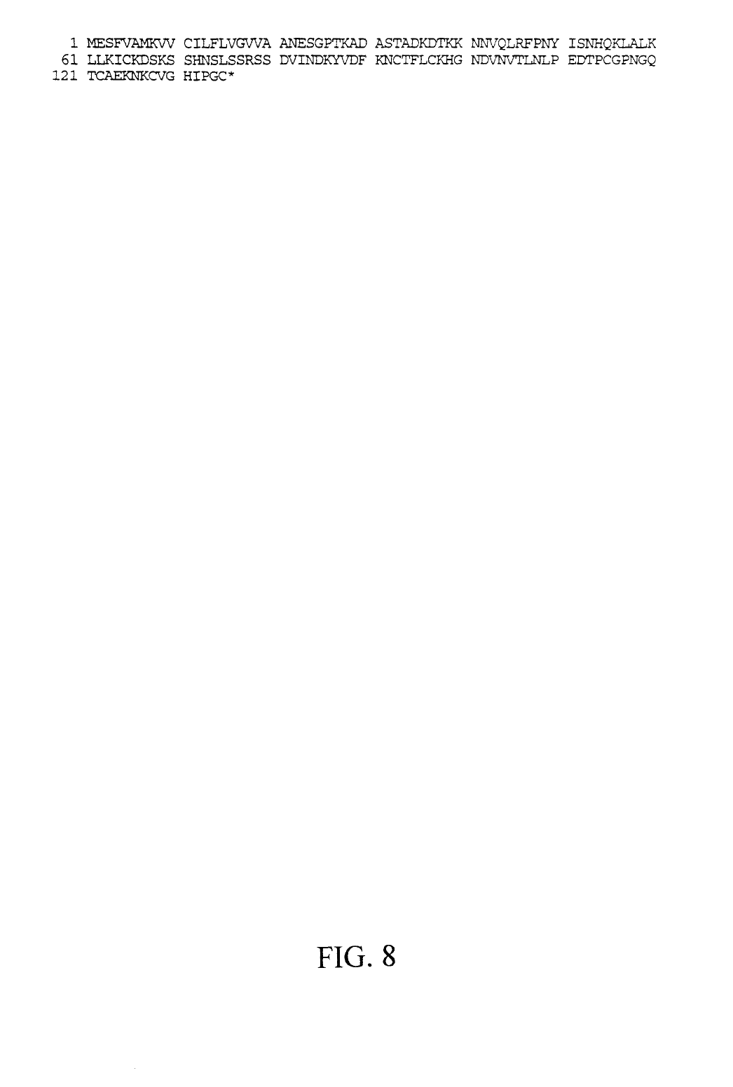

A seventh clone contained a 408 bp open reading frame that encodes a 135-amino-acid polypeptide with an expected molecular weight of 14.7 kDa, a pI of 9.7 and a 20-amino-acid signal sequence. We designated the gene as salp15 (FIG. 7 and SEQ ID NO: 7) and the protein encoded by the gene as Salp15. The deduced amino-acid sequence of the Salp15 polypeptide is set forth in FIG. 8 and SEQ ID NO: 8. We performed a BLAST search of the Genbank database which revealed weak homology to the active motif regions of Inhibin A, a member of the TGF-β superfamily.

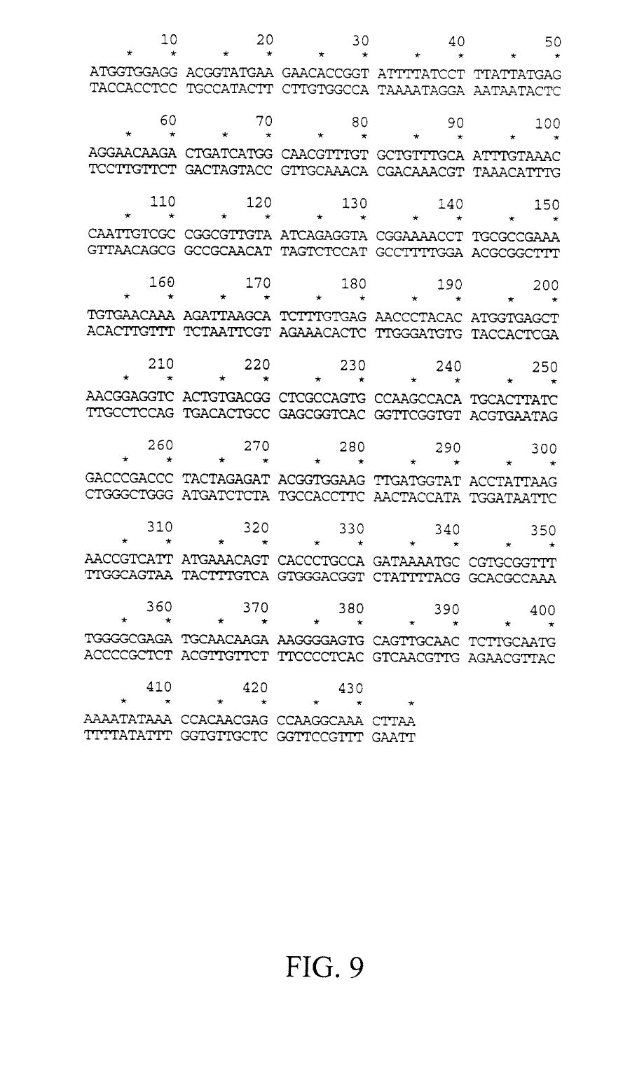

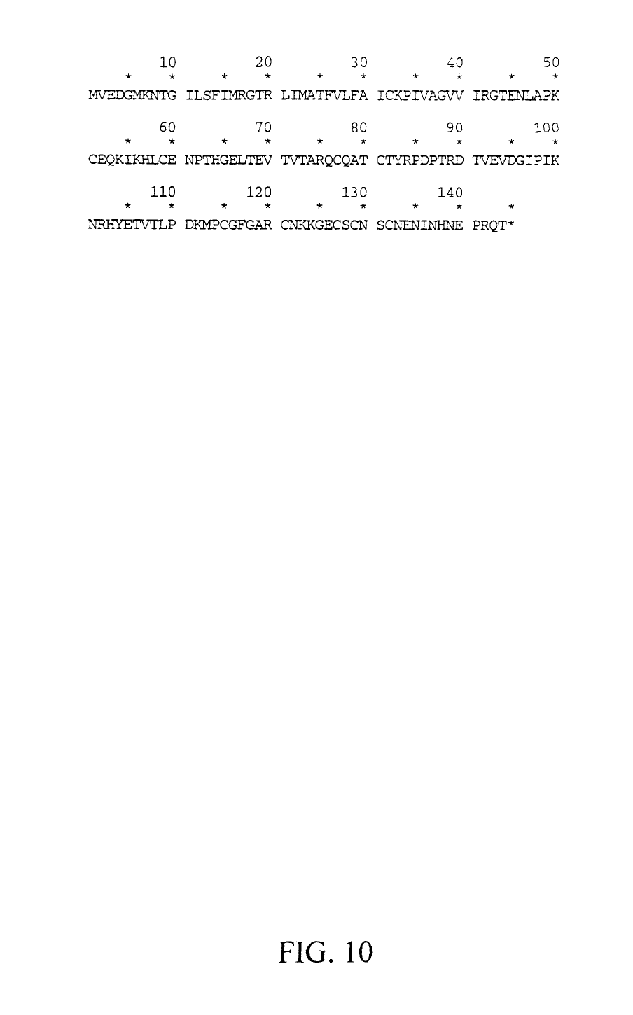

An eighth clone contained a 435 bp open reading frame that encodes a 144-amino-acid polypeptide with an expected molecular weight of 16.0 kDa, a pI of 8.9 and a 38-amino-acid signal sequence. We designated the gene as salp16A (FIG. 9 and SEQ ID NO: 9) and the protein encoded by the gene as Salp16A. The deduced amino-acid sequence of the Salp16A polypeptide is set forth in FIG. 10 and SEQ ID NO: 10. This protein is different from Salp16 [Das et al., “Salp16, a gene induced in Ixodes scapularis salivary glands during tick feeding,” Am. J. Trop. Med. Hyq., 60, pp. 99-106 (2000)] and is therefore named Salp16A.

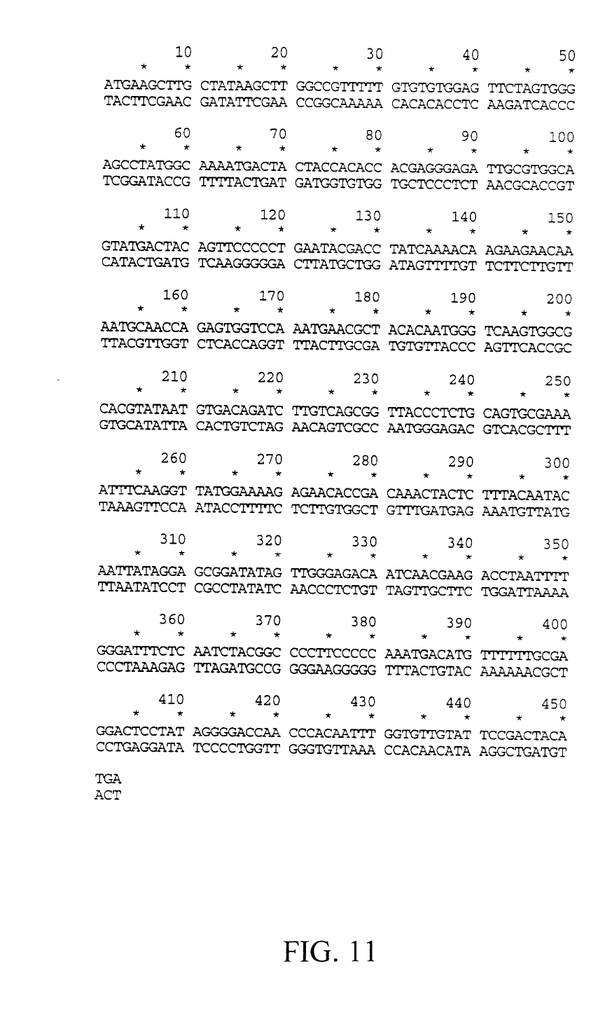

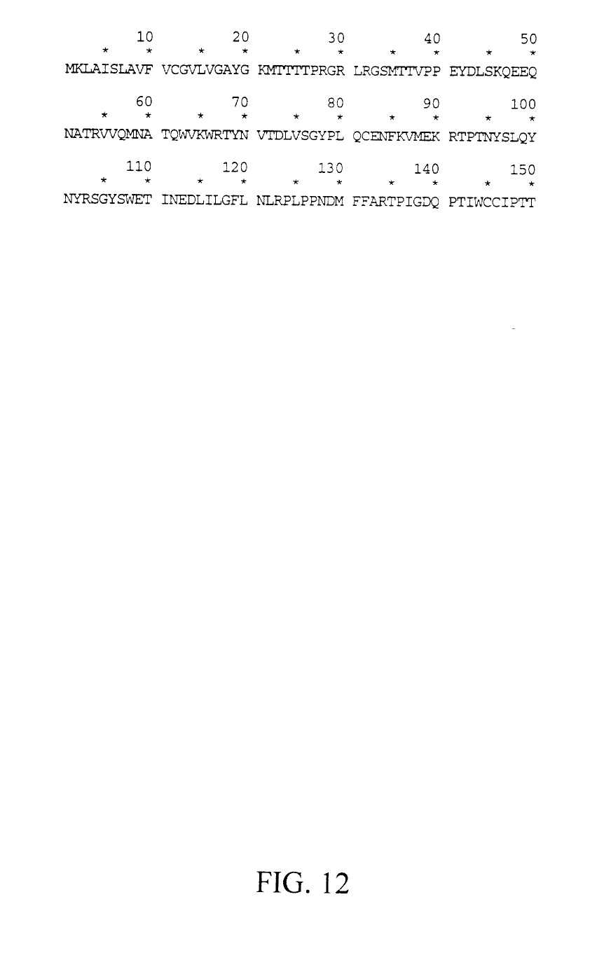

A ninth clone contained a 453 bp open reading frame that encodes a 150-amino-acid polypeptide with an expected molecular weight of 17.2 kDa, a pI of 9.2 and a 20-amino-acid signal sequence. We designated the gene as salp17 (FIG. 11 and SEQ ID NO: 11) and the protein encoded by the gene as Salp17. The deduced amino-acid sequence of the Salp17 polypeptide is set forth in FIG. 12 and SEQ ID NO: 12.

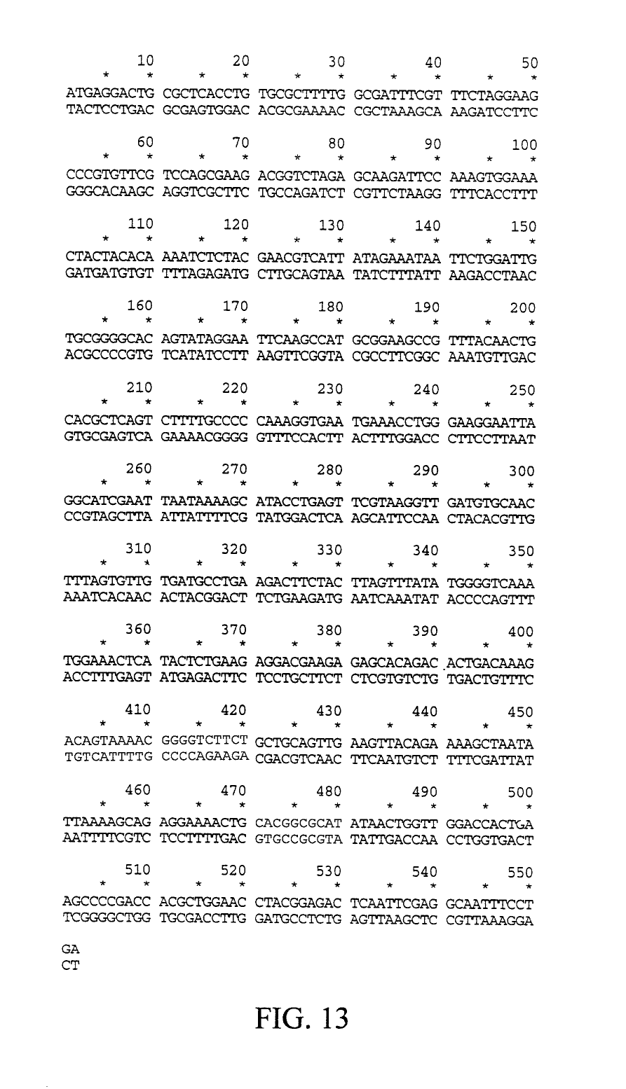

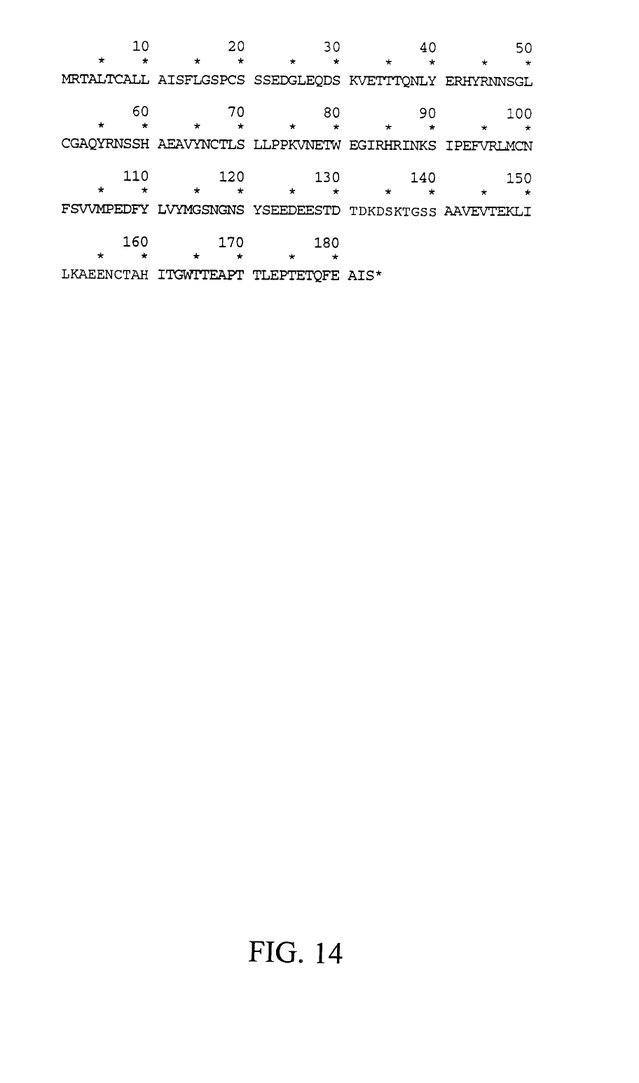

A tenth clone contained a 552 bp open reading frame that encodes a 183-amino-acid polypeptide with an expected molecular weight of 20.4 kDa, a pI of 4.5 and a 22-amino-acid signal sequence. We designated the gene as salp20 (FIG. 13 and SEQ ID NO: 13) and the protein encoded by the gene as Salp20 . The deduced amino-acid sequence of the Salp20 polypeptide is set forth in FIG. 14 and SEQ ID NO: 14.

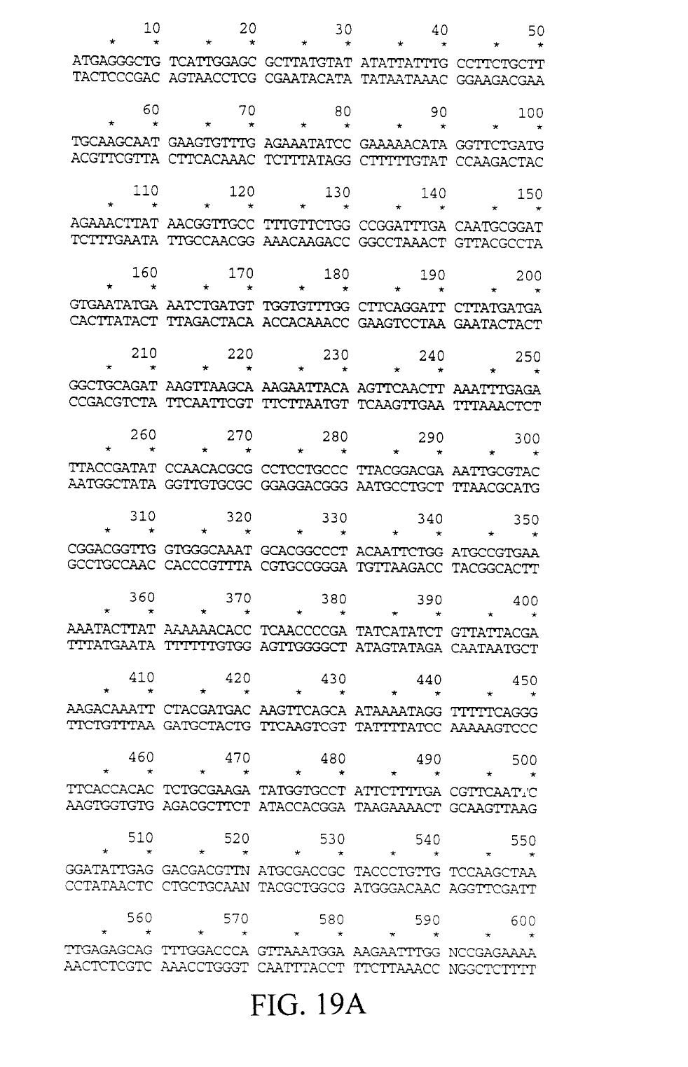

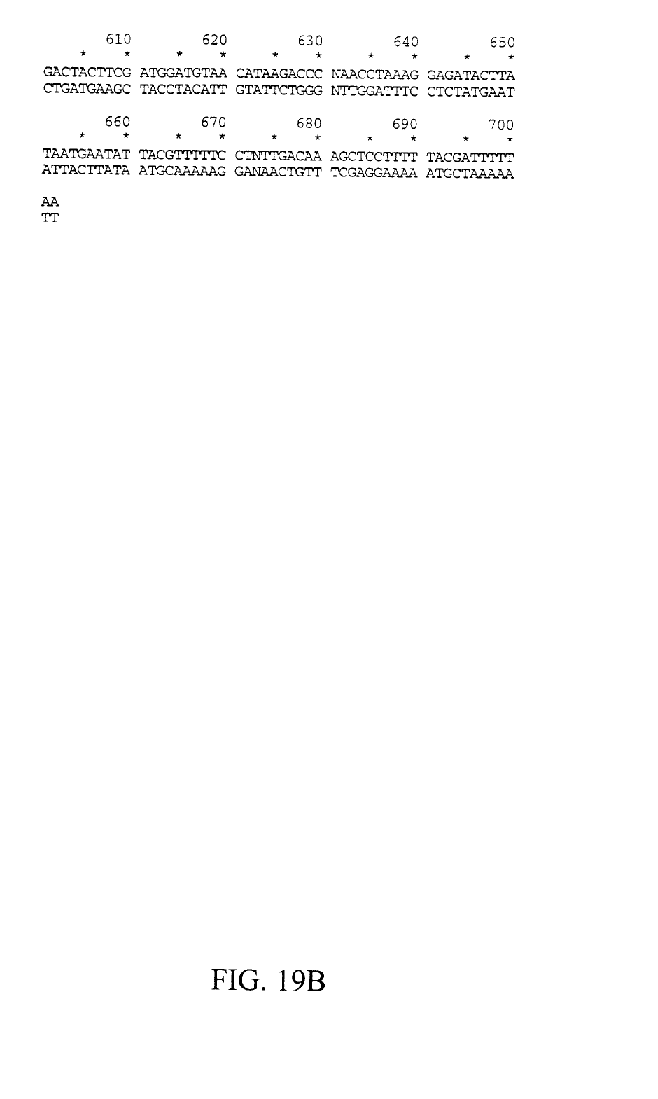

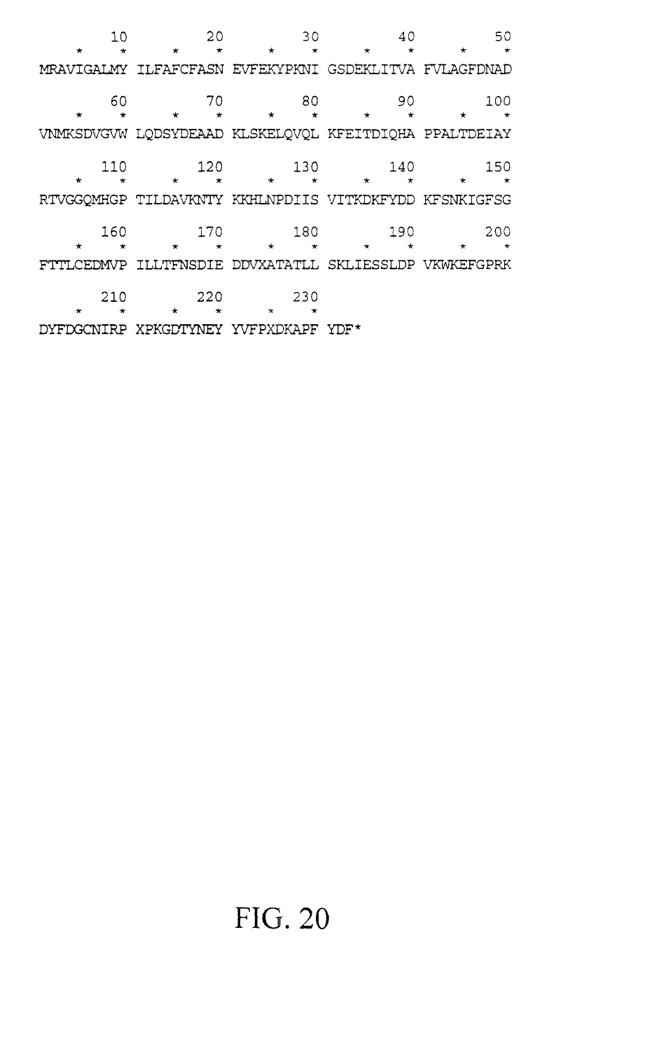

An eleventh clone contained a 702 bp open reading frame that encodes a 233-amino-acid polypeptide with an expected molecular weight of 26.4 kDa, a pI of 4.4 and an 18-amino-acid signal sequence. We designated the gene as salp26A (FIGS. 19A-19B and SEQ ID NO: 19) and the protein encoded by the gene as Salp26A. The deduced amino-acid sequence of the Salp26A polypeptide is set forth in FIG. 20 and SEQ ID NO: 20.

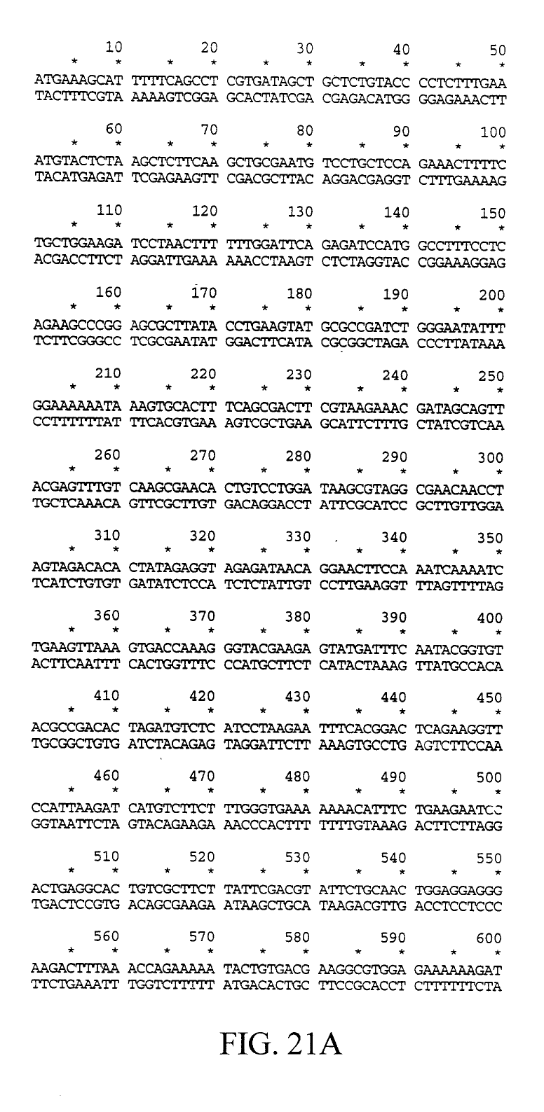

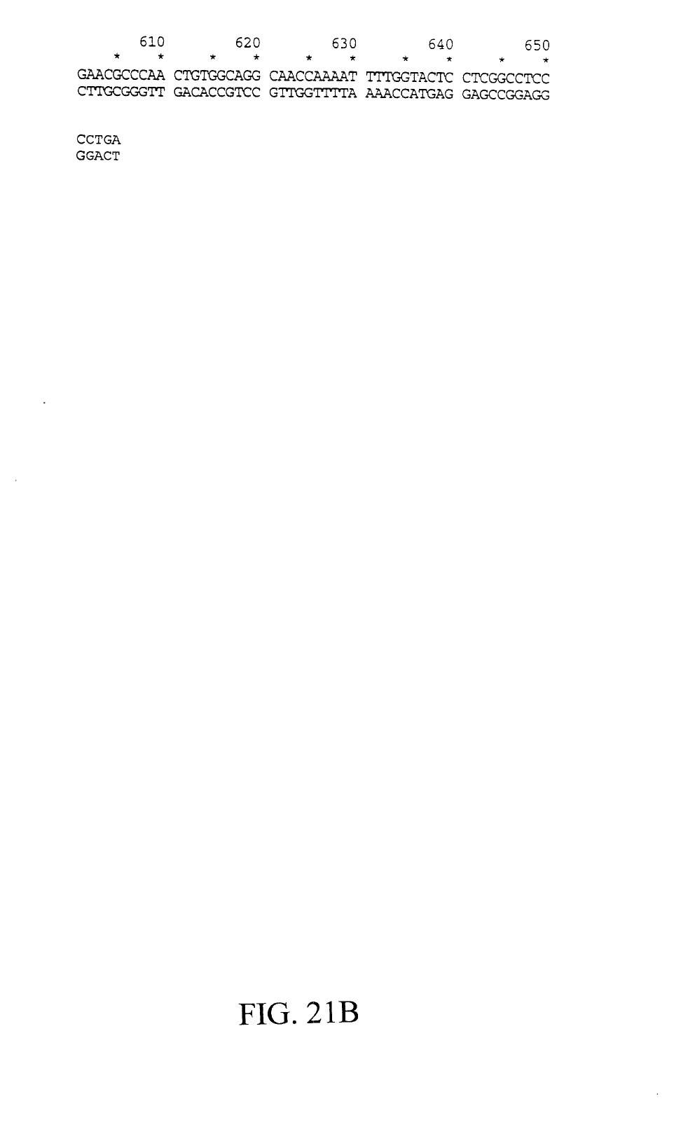

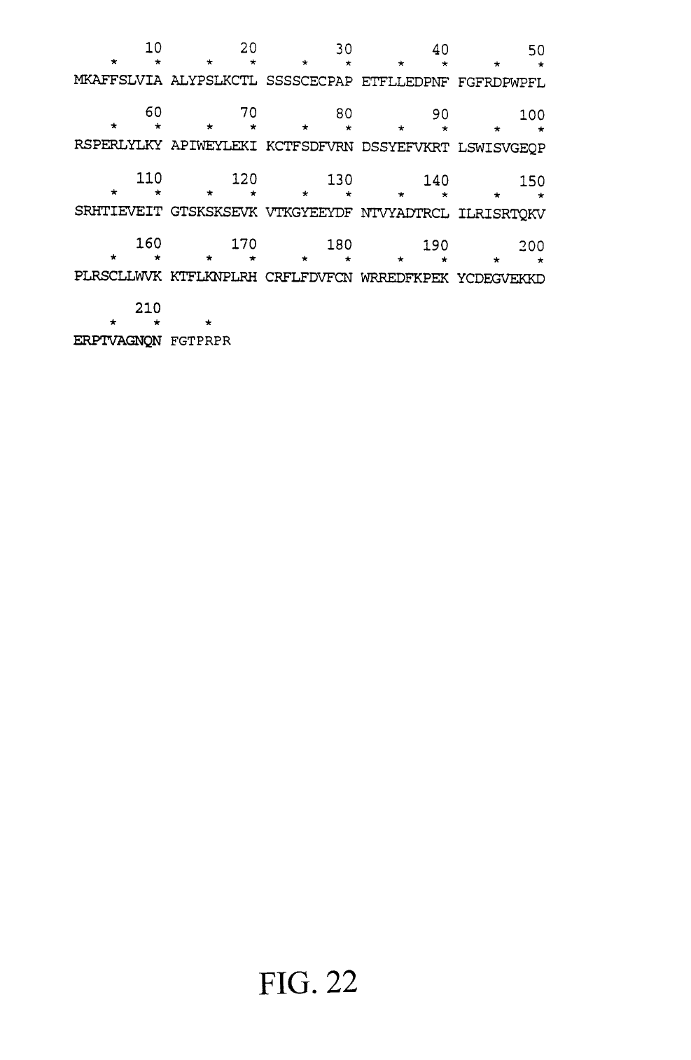

A twelfth clone contained a 657 bp open reading frame that encodes a 218-amino-acid polypeptide with an expected molecular weight of 25.7 kDa, a pI of 9.4 and an 18-amino-acid signal sequence. We designated the gene as salp26B (FIGS. 21A-21B and SEQ ID NO: 21) and the protein encoded by the gene as Salp26B. The deduced amino-acid sequence of the Salp26B polypeptide is set forth in FIG. 22 and SEQ ID NO: 22.

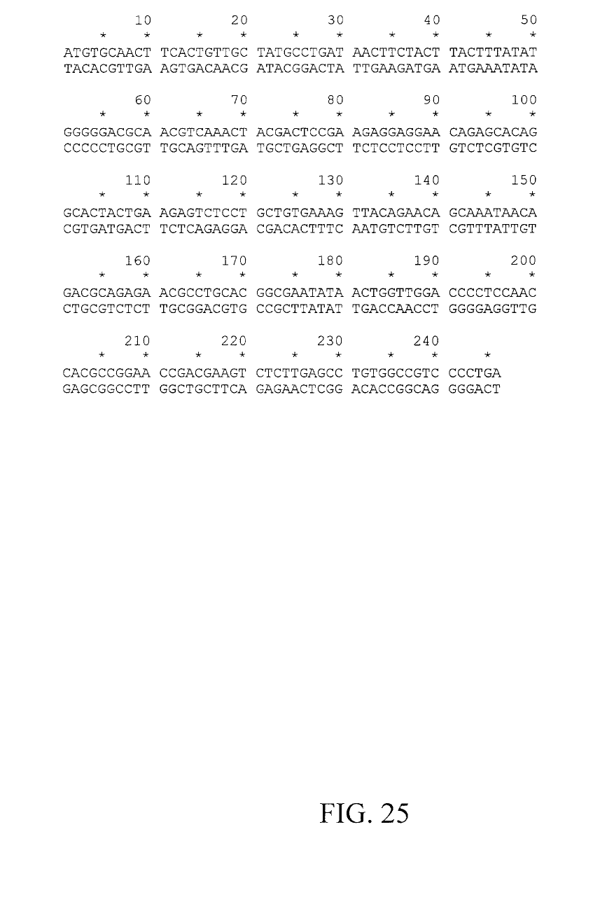

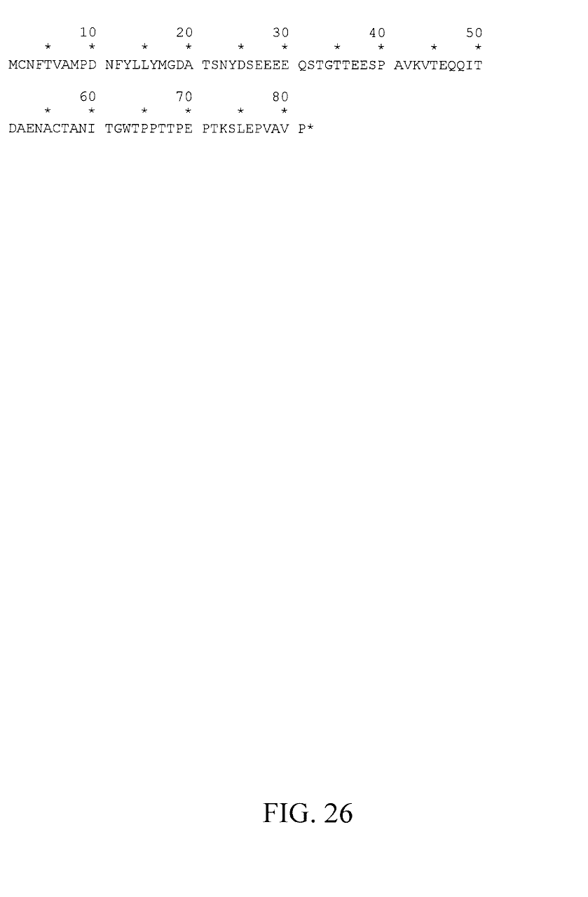

A thirteenth clone contained a 243 bp open reading frame that encodes a 81-amino-acid polypeptide with an expected molecular weight of 8.8 kDa, a pI of 9.5 and an 18-amino-acid signal sequence. We designated the gene as salp9 (FIG. 25 and SEQ ID NO: 25) and the protein encoded by the gene as Salp9. The deduced amino-acid sequence of the Salp9 polypeptide is set forth in FIG. 26 and SEQ ID NO: 26.

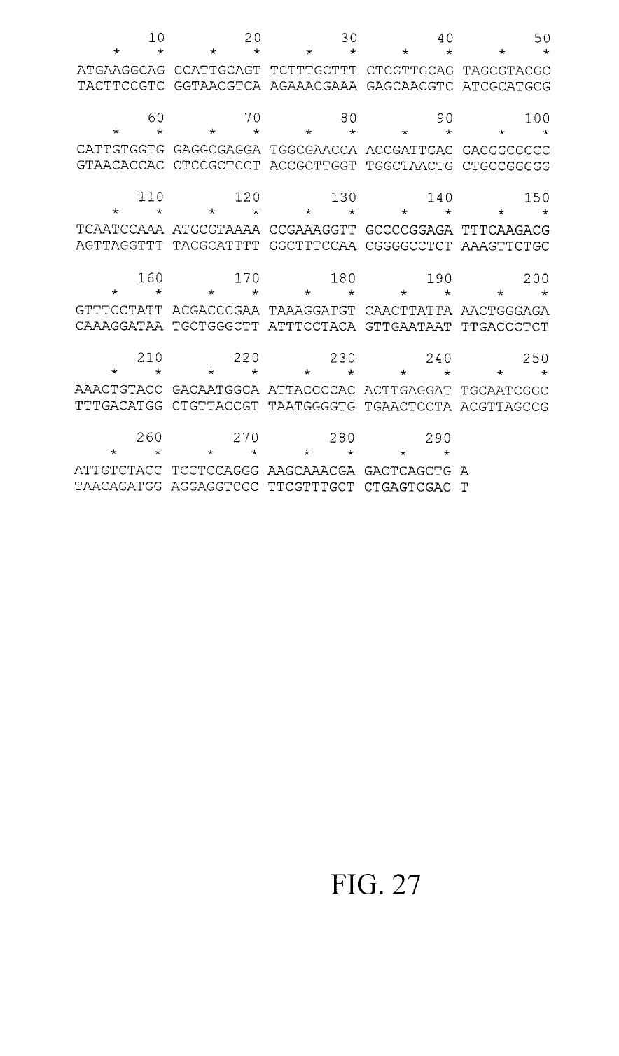

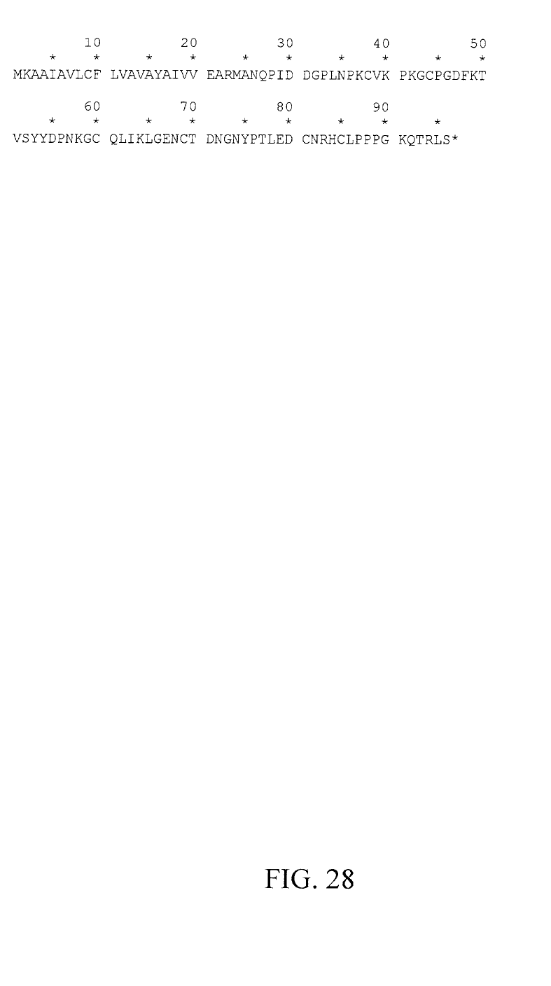

A fourteenth clone contained a 288 bp open reading frame that encodes a 96-amino-acid polypeptide with an expected molecular weight of 10.4 kDa, a pI of 8.9 and a 17-amino-acid signal sequence. We designated the gene as salp10 (FIG. 27 and SEQ ID NO: 27) and the protein encoded by the gene as Salp10. The deduced amino-acid sequence of the Salp10 polypeptide is set forth in FIG. 28 and SEQ ID NO: 28. We performed a BLAST search of the Genbank database which revealed homology with several Kunitz-type protease inhibitors and coagulation inhibitors.

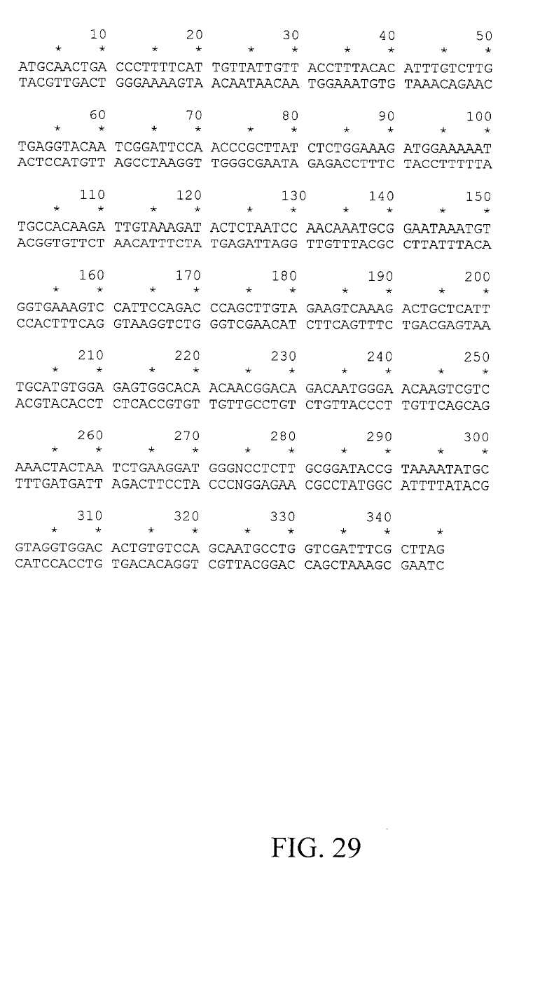

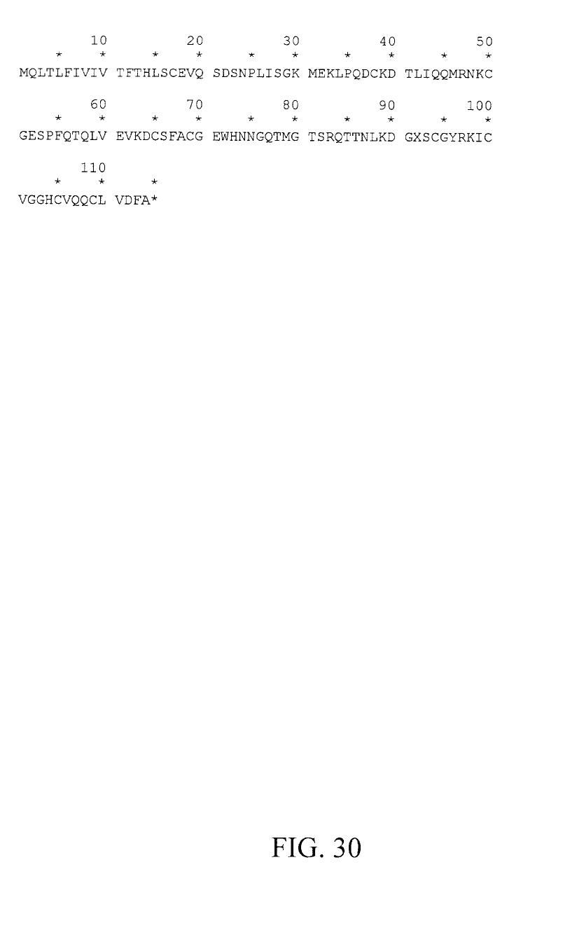

Finally, the fifteenth clone contained a 342 bp open reading frame that encodes a 114-amino-acid polypeptide with an expected molecular weight of 12.7 kDa, a pI of 6.3 and a 21-amino-acid signal sequence. We designated the gene as salp13 (FIG. 29 and SEQ ID NO: 29) and the protein encoded by the gene as Salp13. The deduced amino-acid sequence of the Salp13 polypeptide is set forth in FIG. 30 and SEQ ID NO: 20. We performed a BLAST search of the Genbank database which revealed very weak similarity with the TGF-β superfamily of proteins.

All of the sequences had poly A tails, indicating active expression of these genes in the salivary gland.

### EXAMPLE II

### Identification of I. scapularis Salivary Gland Antigens by 2-D Gel Electrophoresis

We performed two-dimensional gel electrophoresis to directly identify antigenic I. scapularis salivary gland components. We dissected 50 pairs of salivary glands from engorged nymphs and boiled them in sample buffer. We then separated the proteins in these samples on a two-dimensional gel electrophoresis apparatus by molecular mass and pI. We then transferred the proteins to nitrocellulose membranes and probed the blots with tick-immune or normal (control) rabbit sera.

Compared to the control sera, tick-immune sera recognized several protein spots in the range of 26 kDa to 100 kDa, among which a large spot at about 26 kDa was very conspicuous. We cut this approximately 26 kDa out of the denaturing gel, digested the protein with trypsin and sequenced the resulting fragments by Edman degradation. One such fragment yielded the sequence FFFENGEK (SEQ ID NO. 31) and appeared to be a fragment of Salp14. We used this sequence to design and synthesize degenerate primers. We then used these primers to perform 3′ RACE according to the manufacturer's instructions (Clontech Laboratories, Inc., Palo Alto, Calif.) with freshly isolated RNA from engorged nymphal salivary glands. These experiments confirmed that our cDNA clone of salp14 corresponds to the full-length RNA transcript.

We employ this method to confirm that our other clones are full-length as well. We design primers specific to the 3′ end of the transcript and perform 3′ RACE as described above.

### EXAMPLE III

### Recombinant Expression of Tick Antigens

We expressed I. scapularis salivary gland antigens Salp25C, Salp14A, Salp14B, Salp15 and Salp20 as recombinant fusion proteins in an E. coli expression system using pBAD-TOPO™-Thiofusion expression kit (Invitrogen™, Carlsbad, Calif.) In this system, the protein of interest is fused at the carboxy-terminal of a modified histidine-patch thioredoxin (TR) and the expression is under control of a tightly regulated araBAD promoter which is induced by arabinose. The fusion protein also contains a carboxy-terminal V5 epitope for easier identification by antibody and six consecutive histidines for affinity purification of the protein in a metal-chelating column chromatography. The following protocol was used to express and purify the TR-fused recombinant proteins.

Generation of PCR Fragments for salp25C, salp14A, salp14B, Salp15 and salp20 Genes

Specific primer sets were designed for each gene and synthesized in the DNA synthesis laboratory of the Critical Technology Division of the Department Of Pathology of Yale University School of Medicine. For salp25C, we amplified the full-length gene excluding the stop codon at the end. For the other four genes, we excluded the N-terminal secretory signal sequence (1-54 bases) and the stop codon during amplification. The pBluescript® vector containing the gene inserts (which were obtained after excision of the Lambda Zap® II cDNA library screening) were used as the individual DNA template for the PCR reactions.

We conducted a 50 μl PCR reaction using DNA template (10 ng), 10×PCR Buffer (5 μl), 50 mM dNTPs (0.5 μl), primers (1 μM each), sterile water to a final volume of 49 μl and Taq Polymerase (1 unit/μl) (1 μl).

The PCR reaction was performed in a thermal cycler for 30 cycles using 1 min at 94° C. for denaturation, 1 min at 55° C. for annealing and 2 min at 72° C. for extension. We purified the PCR product by agarose gel electrophoresis.

For the 5 μl TOPO™ cloning reaction, we used 2 μl of PCR product, sterile water added to a final volume of 4 μl and pBAD/Thio-TOPO™ vector (1 μl). We incubated the reaction mix for 5 min at room temperature.

Transformation Reaction

We added 2 μl of 0.5 M β-mercaptoethanol followed by 2 μl of the TOPO™ cloning reaction (see above) to each vial of competent cells and mixed. We incubated the mixture on ice for 30 min, then heat-shocked the cells for 30 sec at 42° C. We added 250 μl of room temperature SOC medium to the cells and incubated at 37° C. for 30 min.

We spread 50 μl from each transformation on a prewarmed plate and incubated overnight at 37° C. We directly checked ten positive clones from each transformation for expression of the fusion proteins.

Expression of Fusion Proteins

For each transformant or control, we inoculated 2 ml of Luria broth (LB) containing 50 μg/ml ampicillin with a single recombinant E. coli colony. We grew the cultures overnight at 37° C. with shaking. The next day, we inoculated 10 ml of LB containing 50 μg/ml ampicillin with 0.1 ml of the overnight culture. We incubated the cultures at 37° C. with vigorous shaking to an OD₆₀₀=˜0.5. At this point (which was the zero point), we took a 1 ml aliquot (without the recombinant protein expression). We then added 90 μl 20% arabinose to induce the cells for fusion protein synthesis. The cells were induced for 4 hr. We then pelleted and lysed the cells for protein purification by metal-chelating column chromatography.

Those of skill in the art will recognize that additional tick antigens can be isolated using the methods described herein. Recombinant antigen can be purified in a number of ways. For example, recombinant antigen can be expressed as a GST-fusion protein and purified using thrombin to cleave at a thrombin cleavage site located between the GST and the recombinant tick antigen. Finally, recombinant antigens can be recovered by equilibrium dialysis after purification of the antigen from SDS-PAGE gels.

### EXAMPLE IV

### In vivo Expression of the salp Genes

To determine whether the genes identified in screening of the salivary gland library were induced by feeding, we performed RT-PCR. We isolated total RNA from 100 pairs of unfed nymphs and 20 salivary glands of partially engorged nymphs (after feeding 66 hrs). We made cDNA from 1 μg of each RNA sample using reverse transcriptase (RT) and oligo dT as the primer. As a negative control to exclude the possibility of DNA contamination, we always performed an experiment where RT was not added to the cDNA synthesis reaction for each sample. We then used 2 μg of cDNA and salp gene-specific primers (5′, bases 1-23; 3′, last 23 bases) to amplify the cDNA encoding the proteins of the invention from the cDNAs made from RNA isolated from both unfed and partially engorged nymphs. We tested 14 of the 15 genes and found that three of them (salp20, Salp25C and salp26B) were expressed in unengorged salivary glands, whereas all 14 genes tested were expressed in engorged glands. We observed that although salp20 was detected in unfed nymphs, its expression increased in engorged nymphs. In contrast, the expression levels of the other genes that are expressed in unfed nymphs, Salp25C and salp26D, appeared similar in the salivary glands of both unengorged and engorged nymphs.

### EXAMPLE V

### Active Immunization

To test the tick polypeptides of the invention for the ability to confer tick immunity, we immunized naive guinea pigs with a combination of thioredoxin(TR)-Salp25C and TR-Salp14B in incomplete Freund's adjuvant (IFA). We boosted twice at 15-day intervals. Fourteen days after the last boost, we place 50 I. scapularis ticks on the shaved backs of the animals immunized with the fusion proteins. For a positive control, we challenged animals made tick-immune as described in Example I. As a negative control, we challenged animals that had been immunized with thioredoxin in IFA. We judged tick immunity by the duration of tick attachment, engorgement weight and mortality during engorgement.

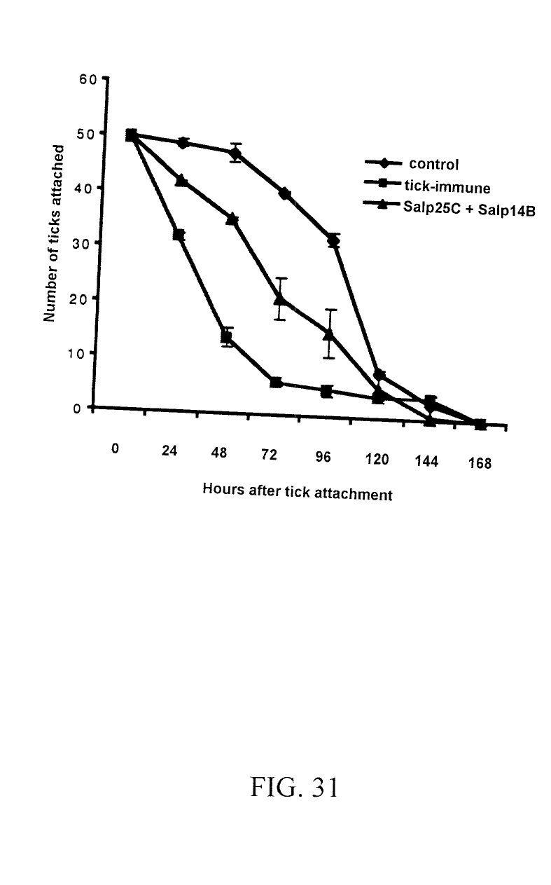

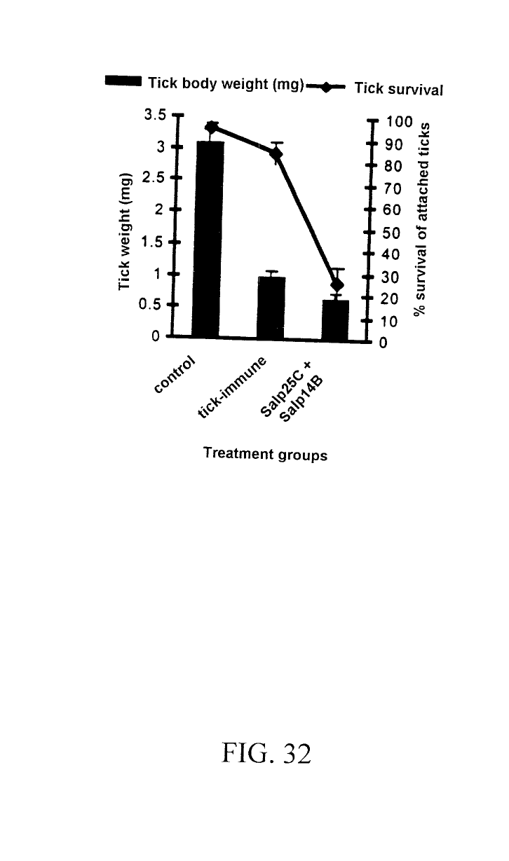

The results of this active immunization are depicted graphically in FIGS. 31 and 32. As shown in FIG. 31, the average duration of stay of the ticks on guinea pigs immunized with the combination of Salp25C and Salp14B was significantly diminished compared to control animals. Likewise, as shown in FIG. 32, the engorgement weight of ticks feeding on those animals also was significantly reduced and most of the ticks died on the guinea pig. Accordingly, the data presented here indicates that immunization with a combination of Salp25C and Salp14B is sufficient to confer tick immunity in an immunized animal.

### EXAMPLE VI

### Passive Immunization

We obtained tick-immune sera from rabbits and guinea pigs by exposure to tick feeding as described in Example I. We confirmed that both tick-immune rabbits and guinea pigs developed antibodies to I. scapularis antigens, which were detectable by ELISA at a serum dilution of 1:1,000 using tick saliva as a substrate. We then transferred sera from tick-immune animals (rabbits or guinea pigs) to naive animals. We then infested these animals with I. scapularis nymphs and monitored the progress of tick infestation. We observed that passive transfer of immune sera partially protected either guinea pigs or rabbits from tick infestation (FIGS. 37A-D, P<0.05, Students' t-test). This effect was not as strong as seen with active immunization with either the tick antigens of this invention or following tick infestation. We also observed that animals that were passively administered immune sera also developed erythema at the sites of tick attachment. The erythema at these sites was similar in intensity to the erythema observed following active sensitization (FIG. 37C and FIG. 37D).

We also passively immunize animals using antiserum from animals immunized with recombinant tick polypeptides of the invention. We prepare antiserum by immunizing C3H/He mice with recombinant tick polypeptides of the invention and boost twice. Fourteen days after the last boost, we sacrifice the immunized animals and collected the antiserum. We immunize guinea pigs with the antiserum, challenge the passively immunized animals with ticks and evaluate tick immunity as described above.

### EXAMPLE VII

### Preparation of Fab Fragments of Immune Serum

To obtain Fab fragments of immune serum for use in screening a salivary gland expression library, we first make rabbit and/or guinea pig anti-tick antiserum. We repeatedly infest rabbits and/or guinea pigs with larval or nymphal ticks, preferably I. scapularis ticks. We determine if the animals are tick-immune if the site of tick attachment becomes red or if tick feeding is less than 48 hours (see Example VI). We bleed tick-immune animals to collect tick-immune serum.

We also prepare guinea pig anti-tick salivary gland antiserum by immunizing guinea pigs subcutaneously with salivary-gland extract prepared as described above, in incomplete Freund's adjuvant. We boost twice with the same amount of crude extract.

To prepare the Fab fragment, we precipitate the antiserum with ammonium sulfate and isolate the IgG fraction using DEAE chromatography. We digest the IgG preparation using a solid-phase papain column. We purify Fab fragments from the papain digestion using a protein A affinity column to remove Fc and intact IgG molecules.

### EXAMPLE VIII

### Prevention of Tick Pathogen Transmission

We test the effect of immunization with tick polypeptides of the invention on the transmission of tick-borne pathogens, including but not limited to B. burgdorferi, the agent of human granulocytic ehrlichiosis (aoHGE), Babesia microti, or various Rickettsiae.

Before testing the transmission of B. burgdorferi, the agent of Lyme Disease, we determined whether guinea pigs could be infected by challenge with B. burgdorferi infected ticks. We challenged naive guinea pigs with 5 B31- or N40-strain-infected I. scapularis nymphs. Skin punches at the site of tick attachment and elsewhere 2, 4 and 7 weeks after tick challenge were consistently positive for spirochetes by culture.

To confirm infection, we determined that guinea pigs develop an immune response against B. burgdorferi. western blots of cloned N40 spirochetes probed with serum from the challenged animals showed antibodies to flagellin, P39 and OspC antigens. Sera from animal exposed to uninfected ticks and those exposed to infected ticks but that were not culture positive failed to develop such antibodies.

We demonstrated, thus, that guinea pigs become infected with B. burgdorferi by tick challenge.

We then determine if immunization with tick polypeptides or antibodies of the invention affect the transmission of B. burgdorferi. We immunize guinea pigs with tick polypeptides or antibodies of the invention as described above and five weeks later, we challenge the immunized animals with ticks from a pool with an approximately 80% infection rate of B. burgdorferi. We obtain skin-punch biopsies at the tick attachment site and serum samples at 2, 4 and 7 weeks after tick challenge. At 8 weeks after challenge we sacrifice the animals and collect blood, bladder and spleen for culture.

One of skill in the art will appreciate that the ability of the tick polypeptides or antibodies of the invention to prevent of lessen the transmission of other tick born pathogens can be determined using the method described herein.

### EXAMPLE IX

### Preparation of Antibodies to Tick Polypeptides

To prepare antibodies to a tick polypeptide of the invention, we immunize C3H/He mice subcutaneously with TR-Salp25C or TR-Salp14B in complete Freund's adjuvant and boost with the same amount in incomplete Freund's adjuvant at 14 and 28 days. We immunize control animals in the same manner with either TR or bovine serum albumin (BSA).

Ten days after the last boost, we collect sera from the immunized animals and use it to hybridize to western blots of SDS-PAGE gels of I. scapularis tick extract or to the recombinant polypeptide. We detect binding with alkaline phosphatase goat-anti-mouse antibody developed with nitroblue tetrazolium and 5-bromo-4-chloroindoyl phosphate. Alternatively, we use the ECL™ kit (Amersham, Arlington Heights, Ill.) in which the secondary antibody, horseradish peroxidase-labeled goat anti-mouse antibody, can be detected.

To prepare a monoclonal antibody, we recover antibody-producing cells from the spleens of the immunized animals and fuse the antibody-producing cells with immortalized cells to produce hybridomas according to the methods of Kohler and Milstein. We screen the resulting hybridomas for specific binding to Salp25C or Salp14B. Those of skill in the art will appreciate that polyclonal and monoclonal antibodies specific for the other tick polypeptides of the invention may be prepared using the methods described herein.

### EXAMPLE X

### Salp15 Inhibits IL-2 Production

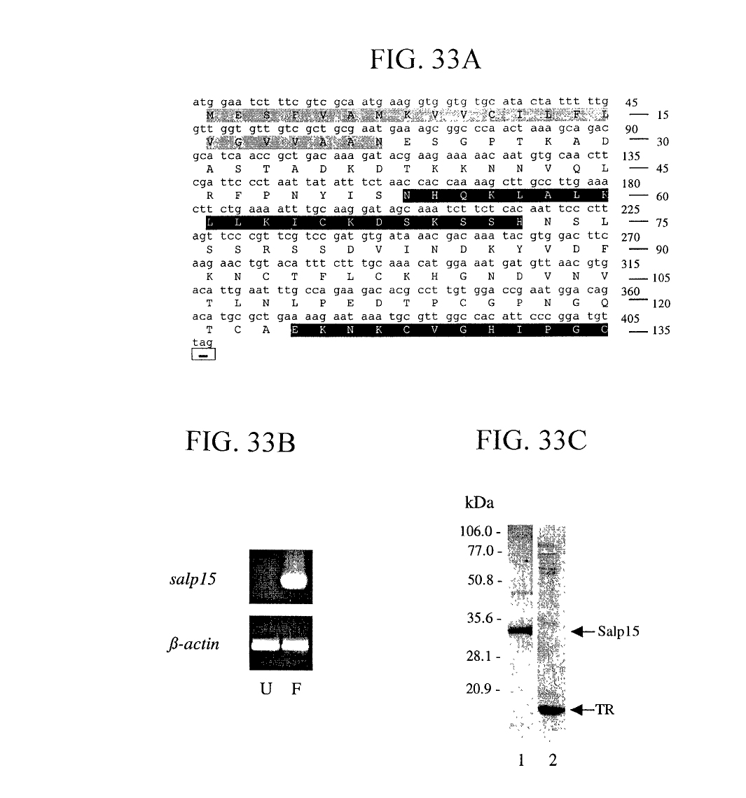

We investigated the effect of Salp15 on IL-2 production by CD4⁺ T cells because Salp15 has some sequence similarity with two motifs of Inhibin A, a member of the TGF-β superfamily (FIG. 33A). We purified CD4⁺ T cells from the spleens of 6- to 8-week-old mice by negative selection using antibodies specific for CD8a, B220, pan NK, Ly6G-GR1, Mac-1 and Class II (BD PharMingen, San Diego, Calif.) and goat anti-rat and goat-anti-mouse IgG bound to magnetic microbeads (Miltenyi Biotec, Auburn, Calif.). We measured the purity of the resulting preparation of CD4⁺ T cells by FACS analysis and found it to be greater than 90% pure. We then activated the CD4⁺ T cells by placing them at a concentration of 10⁶/ml in 24-well plates with 5 μg/ml anti-CD3 and 1 μg/ml anti-CD28 in a final volume of 1 ml and incubating for 44 hr. We then added TR-Salp15 or thioredoxin (negative control) to the wells at different concentrations depending on the particular experiment.

We then collected the culture supernatants by centrifugation at 5,000×g for 5 min and determined the levels of different cytokines (IL-4, IFN-γ and IL-2) by Capture ELISA. Briefly, we coated 96-well ELISA plates (ICN Pharmaceuticals Inc., Costa Mesa, Calif.) with 2 μg/ml capture antibody for 2 hr at 37° C. and then blocked the plates overnight at 4° C. with PBS supplemented with 10% fetal calf serum (PBS/FCS). We then applied the samples and incubated at 37° C. for 1 hr. We then washed the plates with PBS supplemented with Tween 20 (0.5% v/v) (PBS/Tween) and added 1 μg/ml biotinylated detection antibody. We quantified cytokine levels by incubating the plates with horseradish-peroxidase-conjugated avidin and adding a substrate for the enzyme (TMB, Kirkegaard and Perry Laboratories, Inc., Gaithersburg, Md.) followed by the stop solution (TMB 1 Component Stop Solution, Kirkegaard and Perry Laboratories, Inc.). We then compared the values we obtained with our experimental samples to values obtained using standard concentrations of recombinant mouse IL-2, IL-4 and IFN-γ (BD PharMingen).

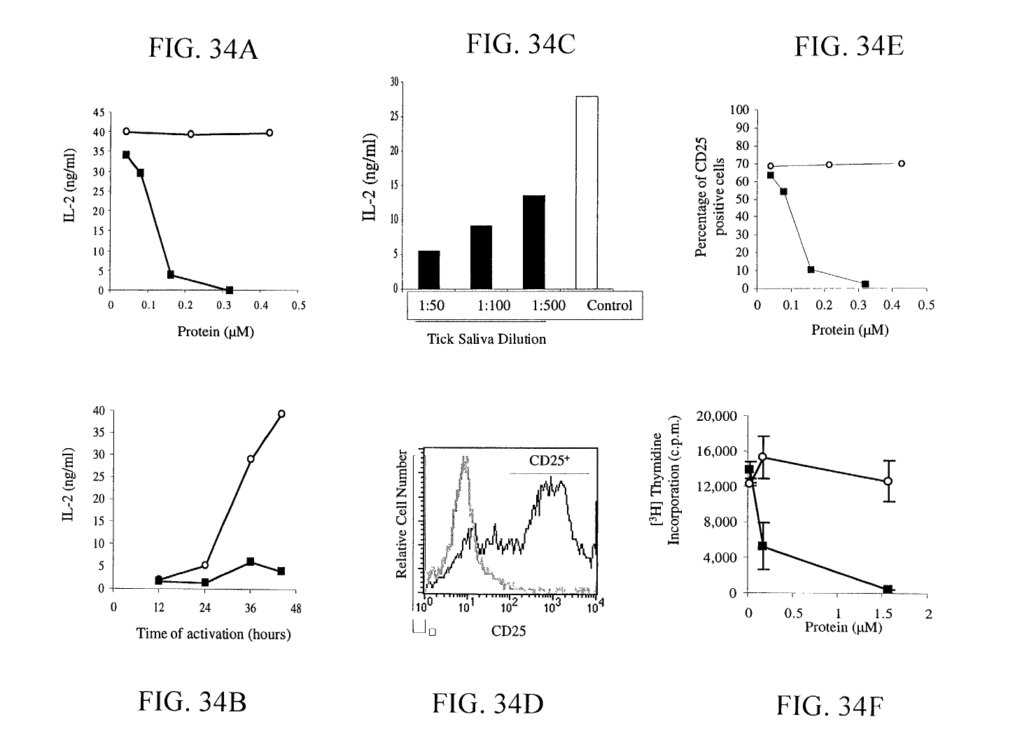

We observed that the level of IL-2 produced by CD4⁺ T cells stimulated in the presence of Salp15 was lower than with the control antigen, TR (FIG. 34A). We found that the inhibitory effect of Salp15 on CD4⁺ T cell IL-2 production was dose-dependent (FIG. 34A) and evident as soon as 12 hours after activation (FIG. 34B). We also tested preparations of tick saliva, which contain many of the polypeptides of the invention, and found that it, too, inhibited IL-2 production by CD4⁺ T cells in response to CD3/CD28 signals (FIG. 34C). This result was in agreement with previous studies using concanavalin A (ConA) activation [Urioste et al., “Saliva of the Lyme Disease Vector, Ixodes dammini, Blocks Cell Activation by a Nonprostaglandin E2-dependent Mechanism,” J. Exp. Med., 180, pp. 1077-1085 (1994)].

### EXAMPLE XI

### Salp15 Inhibits CD25 Expression

We then tested whether we would observe other consequences of reduced IL-2 production by CD4⁺ T cells activated in the presence of Salp15. IL-2 produced by activated CD4⁺ T cells causes upregulation of the α-chain of the IL-2 receptor, CD25, on the surface of T lymphocytes [Demaison et al., “IL-2 Receptor Alpha-chain Expression Is Independently Regulated in Primary and Secondary Lymphoid Organs,” J. Immunol., 161, pp. 1977-1982 (1998)]. To assess whether the inhibitory effect of Salp15 on IL-2 production by CD4⁺ T cells influenced the expression of the α-chain of the IL-2 receptor, we used flow cytometry to measure the percentage of CD25-positive cells. Briefly, we activated 10⁶ cells with anti-CD3/CD28 as described above. At the specified time points, we washed the cells with PBS/Tween and stained them with phycoerythrin (PE) or cy-chrome-conjugated antibodies, which are specific for CD25 and CD4 respectively. We then analyzed the cells by flow cytometry using a FACScalibur apparatus and the CellQuest software package (Becton Dickinson, Mountain View, Calif.). We observed in these experiments that Salp15 also showed an inhibitory effect on CD25 expression (FIG. 34D). Furthermore, this effect was dose-dependent: we observed that increasing molar concentrations of Salp15 during activation decreased CD25 expression on CD4⁺ T cells (FIG. 34E).

### EXAMPLE XII

### Salp15 Impairs the Proliferative Response of CD4⁺ T Cells

Following our observation that Salp15 inhibits both IL-2 production and the expression of the α-chain of the IL-2 receptor on CD4⁺ T cells, we performed proliferation assays in the presence of either Salp15 or TR to test the ability of the cells to proliferate in response to TCR signals. Briefly, we activated cells with 5 μg/ml plate-bound anti-CD3 plus 1 μg/ml anti-CD28 in 96-well plates in a final volume of 100 μl. We incubated the cells for 3 days and then added 1 μCi of [³H]-thymidine to the cultures and incubated for an additional 16-24 hr. Similar to what we observed for IL-2 production and CD25 expression, the proliferative responses were impaired in the presence of different concentrations of Salp15, whereas proliferation of CD4′ T cells which were activated in the presence of equimolar concentrations of TR was not affected (FIG. 34F).

We confirmed that Salp15 did not have a general cytotoxic effect by testing the viability of the cells by trypan blue exlusion. These experiments showed no difference in cell viability between thioredoxin- and Salp15-treated CD4⁺ T cells after 22 and 36 hours of activations. The percentage of live cells was 70% vs. 62% at 22 hours and 66% vs. 62% at 36 hours, respectively. In addition, we tested the survival of a CD4⁺ T cell hybridoma line obtained from an MRL/1 pr/1 pr mouse by antigen-specific cloning. This cell line does not depend for survival on either TCR- or IL-2 receptor-mediated signals. We assessed proliferation with this cell line by incubating the cells for 24 hr in the presence of Salp15 or thioredoxin and then pulsing the cells with 1 μCi [³H]-thymidine for the final four hr of the assay. We observed that Salp15 does not affect the viability of this CD4⁺ T cell hybridoma line confirming that Salp15 does not have a general cytotoxic effect on CD4⁺ T cells and further indicating that Salp15 specifically inhibits IL-2 production, CD25 expression and the proliferation of CD4⁺ T cells activated by anti-CD3/CD28.

### EXAMPLE XIII

### The Immunosuppressive Activity of Salp15 is Mediated Through the Inhibition of IL-2 Production

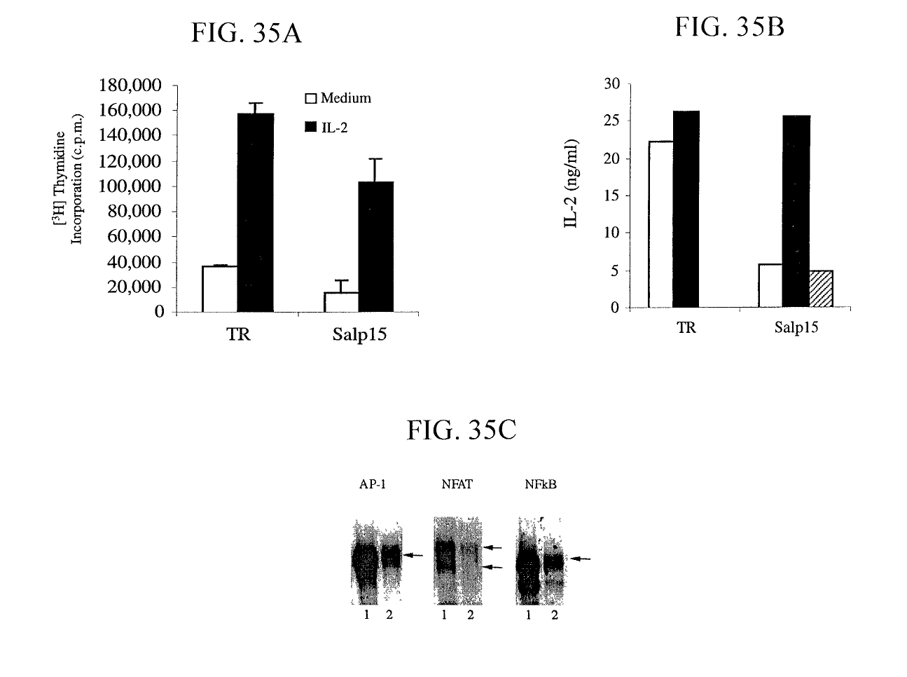

Our results as detailed in Examples X-XII suggested that Salp15 may inhibit CD4⁺ T cell activation through the specific inhibition of IL-2 production, but this effect could also be mediated though IL-2 receptor-derived signals. To determine whether the lack of activation was due to specific inhibition of IL-2 production, we assessed the effect of exogenously added recombinant IL-2 during activation. Briefly, we activated CD4⁺ T cells with anti-CD3/CD28 in the presence of-Salp15 or thioredoxin, as before, but additionally in the presence or absence of 10 ng/ml of recombinant murine IL-2 (rIL-2). We observed that the proliferation of CD4⁺ T cells activated in the presence of Salp15 increased markedly with the addition of 10 ng/ml rIL-2 (FIG. 35A). Furthermore, the expression of CD25 in the presence of either Salp15 or thioredoxin also increased to similar levels in the presence of rIL-2. This result indicated that Salp15 also did not affect the upregulation of the IL-2 receptor under these conditions. These experiments indicated that Salp15 exerts it affect through inhibition of IL-2 production.

The initial production of IL-2 by CD4⁺ T cells that are undergoing activation provides strong signals that permit the activation process to continue and the cells to enter the proliferative cycle. We therefore tested whether the addition of Salp15 would have the same effect on CD4⁺ T cell activation when added at later time points, i.e. after IL-2 was already available to the cell. We incubated CD4⁺ T cells with anti-CD3/CD28 prior to the addition of Salp15 at various time points and then measured IL-2 production at 44 hours. We observed that IL-2 production was decreased when the cells had been activated with anti-CD3/CD28 for 12 hours prior to the addition of Salp15 (FIG. 35B). In contrast, we observed that the levels of IL-2 produced by CD4⁺ T cells were unaffected when the cells were incubated with anti-CD3/CD28 for 24 hours before the addition of Salp15 (FIG. 35B). This result indicated that the levels of IL-2 produced during the initial stages of CD⁺ T cell activation are adequate to induce the upregulation of the high-affinity receptor complex (Demaison, supra), thus allowing the continuation of the activation process. These experiments further indicated that the inhibition of CD4⁺ T cell activation by Salp15 occurred in the initial 24 hr.

### EXAMPLE XIV

### Salp 15 Inhibits AP-1, NF-κB and Predominantly NF-ATDNA Binding Activity

Cross-linking of TCR complexes and CD28 molecules on naive T cells induces multiple intracellular signaling pathways that lead to the activation of specific nuclear transcription factors, including activator protein (AP)-1, nuclear factor of activated T cells (NF-AT) and nuclear factor kappa B (NF-κB) [Jain et al., “Transcriptional Regulation of the IL-2 Gene,” Curr. Opin. Immunol., 7, pp. 333-342 (1995)]. These regulatory elements comprise the minimal inducible promoter region of the IL-2 gene (Jain et al., supra). Following our observation that Salp15 inhibits early events during the activation of CD4′ T cells, we assessed the binding activity of these transcription factors in CD4⁺ T cells that had been activated with anti-CD3/CD28 in the presence of either Salp15 or thioredoxin.

We obtained nuclear extracts from 3×10⁶ CD4′ T cells activated for 12 hr. with anti-CD3/CD28 as previously described [Schreiber et al., “Rapid Detection of Octamer Binding Proteins with ‘Mini-Extracts’ Prepared from a Small Number of Cells,” Nucl. Acids Res., 17, pp. 6419 (1989)]. We then performed binding reactions using 2 μg of nuclear protein in the presence of 5×10⁴ cpm of the specific ³²P-end-labeled double-stranded oligonucleotide for 25 min at 48° C. as previously described (Schreiber et al., supra). We used the following oligonucleotides: IL2-NF-AT, 5′-GCC CAA AGA GGA AAA TTT GTT TCA TAC AG-3 (SEQ ID NO: 32); AP-1, 5′-GTC GAC GTG AGT CAG CGC GC-3′ (SEQ ID NO: 33) [Rincòn et al., “Transcription Mediated by NFAT Is Highly Inducible in Effector CD4⁺ T helper (T_{H}2) Cells But Not T_{H}1 Cells,” Mol. Cell. Biol., 17, pp. 1522-1534 (1998)], and NF-κB, 5′-GAT CAG AGG GGA CTT TCC GAG-3′ (SEQ ID NO: 34) [Millet et al., “Inhibition of NF-kappaB Activity and Enhancement of Apoptosis by the Neuropeptide Calcitonin Gene-related Peptide,” J. Biol. Chem., 275, pp. 15114-15121 (2000)]. We observed that the levels of NF-AT bound to the target labeled DNA probe were severely reduced in CD4⁺ T cells activated in the presence of Salp15 (FIG. 35C). We also observed that the levels of AP-1 and NF-κB that formed complexes with the specific DNA probe were also reduced, although to a lesser extent (FIG. 35C). These experiments indicated that the inhibitory effect of Salp15 on CD4⁺ T cells is predominantly due to the inhibition of binding activity of NF-AT.

### EXAMPLE XV

### Salp15 Inhibits CD4⁺ T Cell Activation in vivo

Activation of naive CD4⁺ T cells requires a rise in IL-2 production, whereas the activation of effector CD4⁺ T cells is less dependent on this cytokine [Yasui et al., “Transcriptional Repression of the IL-2 Gene in T_{H} Cells by ZEB,” J. Immunol., 160, pp. 4433-40 (1998)]. Therefore, we performed restimulation assays with CD4⁺ T cells from thioredoxin-immunized mice to assess whether Salp15 inhibited the activation of effector CD4⁺ T cells. We immunized 6-week old Balb/c mice subcutaneously with thioredoxin in complete Freund's adjuvant (CFA) and recovered the CD4⁺ T cells from the spleens after 11 days. We then restimulated 10⁶ purified CD4⁺ T cells ex vivo for 40 min at 37° C. in the presence of 50 μg/ml Mitomycin C-treated syngeneic antigen-presenting cells. We measured production of IFN-γ and IL-4 in response to Salp15 and thioredoxin by capture ELISA as describe in Example X.

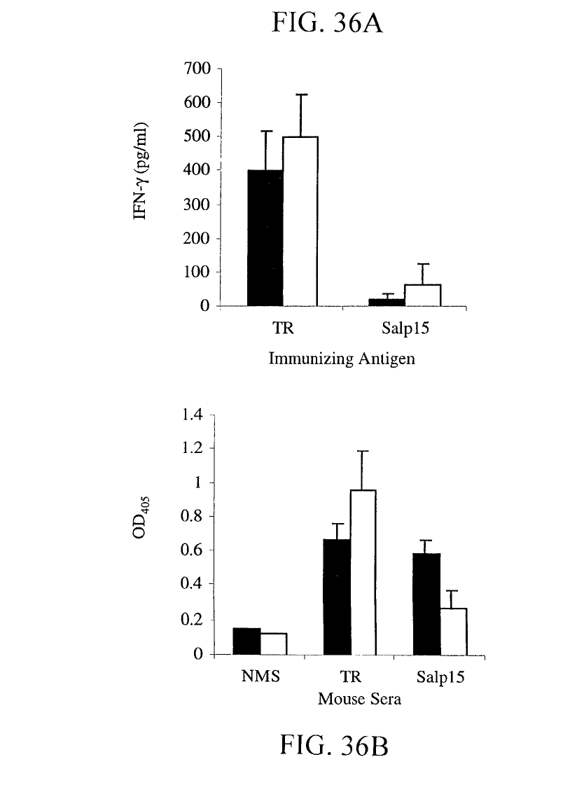

We observed that CD4′ T cells from thioredoxin-immunized animals responded equally to Salp15 and thioredoxin and produced high levels of IFN-γ (FIG. 36A; T test, p=0.591). This indicated that the stimulation of effector CD4⁺ T cells was not affected by the presence of Salp15. We observed that IL-4 production was below detectable levels in all cases.

In order to assess the effect of Salp15 on the murine-immune response in vivo, we repeated the restimulation assays using CD4⁺ T cells from either Salp15- or thioredoxin-immunized Balb/c mice. We immunized the mice with equimolar quantities of Salp15 or thioredoxin, purified CD4⁺ T cells from the spleens of the mice after 11 days and assessed cytokine production ex vivo following restimulation with thioredoxin as before. We observed that CD4⁺ T cells from thioredoxin-immunized mice produced significantly higher levels of IFN-γ than CD4⁺ T cells from Salp15-immunized mice (FIG. 36A; T test, p=0.018). A previous report [Schoeler et al., “Ixodes scapularis: Effects of Repeated Infestations with Pathogen-free Nymphs on Macrophage and T Lymphocyte Cytokine Responses of Balb/c and C3H/HeN Mice,” Exp. Parasitol., 92, pp. 239-248 (1999)] indicated that T lymphocytes from Balb/c mice repeatedly infested with I. scapularis nymphs in vivo do not produce high levels of IFN-γ in response to ConA restimulation ex vivo. Therefore, these data indicate that Salp15 exerts an inhibitory effect on the generation of effector CD4⁺ T cells in vivo.

### EXAMPLE XVI

### Salp15 Inhibits Antibody Production in vivo

CD4⁺ T cells are an important factor in the development of humoral antibody responses and we therefore assessed whether the inhibitory effect of Salp15 on CD4⁺ T cells influenced the generation of the murine antibody response. We immunized Balb/c mice with equimolar quantities of Salp15 and thioredoxin as before. Eleven days later, we analyzed sera from the immunized mice by ELISA for antibodies to thioredoxin. Briefly, we coated ELISA plates (ICN Pharmaceuticals, Inc., Costa Mesa, Calif.) overnight at 4° C. with 1 μg/ml recombinant thioredoxin in coating buffer. We then blocked the plates for two hr at room temperature with PBS/FCS and washed the plates twice with PBS/Tween. We then applied the sera (1:160 dilution for IgM, 1:320 dilution for IgG) and incubated for 1 hr at room temperature. We used sera from naive mice as a control. We washed the wells with PBS/Tween and added alkaline phosphatase-conjugated goat antimouse IgM and IgG (Sigma Chemical Co., Saint Louis, Mo.). After a 1 hr incubation at room temperature, we developed the plates with 1 mg/ml p-nitrophenol phosphate (Sigma Chemical Co.) in glycine buffer.

We found no difference in thioredoxin-specific IgM levels in the sera from Salp15- and thioredoxin-immunized mice (FIG. 36B; T test, p=0.81). This indicated that B-cell responses were not effected by Salp15. In contrast, thioredoxin-specific IgG antibody levels were significantly reduced in the sera obtained from Salp15-immunized mice compared to thioredoxin-immunized mice (FIG. 36B; T test, p=0.03). The impaired development of a strong IgG antibody response to thioredoxin in mice immunized with Salp15 reinforced the idea of an inhibitory effect of the protein on CD4⁺ T cell activation and subsequent T-mediated B-cell help. This result further demonstrated that Salp15 inhibited the murine IgG antibody response to thioredoxin.

### EXAMPLE XVII

### Glutathione Peroxidase Activity of Recombinant I. scapularis Salp25C

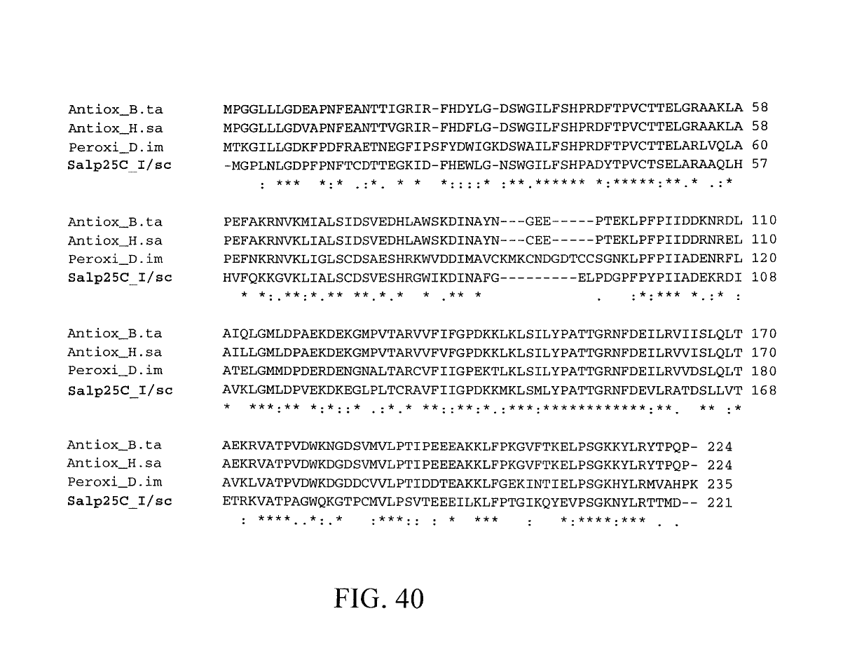

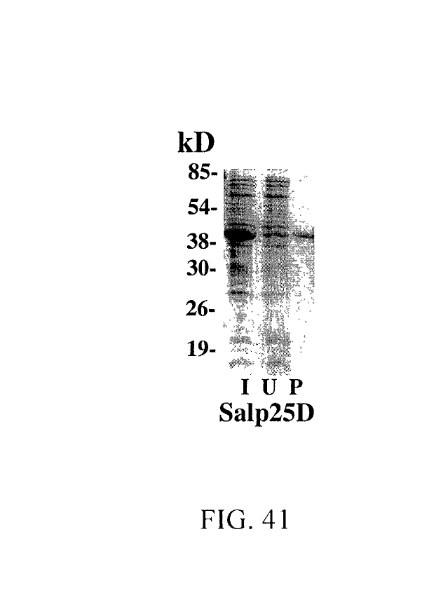

As mentioned, we observed strong sequence similarity between a polypeptide of the invention, Salp25C, and glutathione peroxidases from various eukaryotic organisms (FIG. 40). Therefore, we determined whether Salp25C has glutathione peroxidase activity. We expressed Salp25C using the pBad-TOPO™-Thio expression vector as described in Example III. We isolated the recombinant protein under native conditions. We lysed the cells by incubating for 15 min on ice in 20 mM phosphate buffer (pH 7.8); 500 mM NaCl with 100 μg/ml egg white lysozyme. We then sonicated the cells and then flash froze them in an ethanol-dry ice slurry followed by thawing at 37° C. We repeated this freeze-thaw cycle two more times. We then treated the cell lysates with 5 μg/ml RNase and 5 μg/ml DNase for 15 min at 30° C. We removed insoluble debris by centrifugation at 3,000×g for 15 min. We then passed the clear supernatant over an immobilized-nickel affinity chromatography column and eluted the bound protein with the same buffer at pH 6.0 containing 300 mM imidazole. As shown in FIG. 41, this procedure substantially purified the protein. We then assayed the glutathione peroxidase activity of the purified protein as follows.

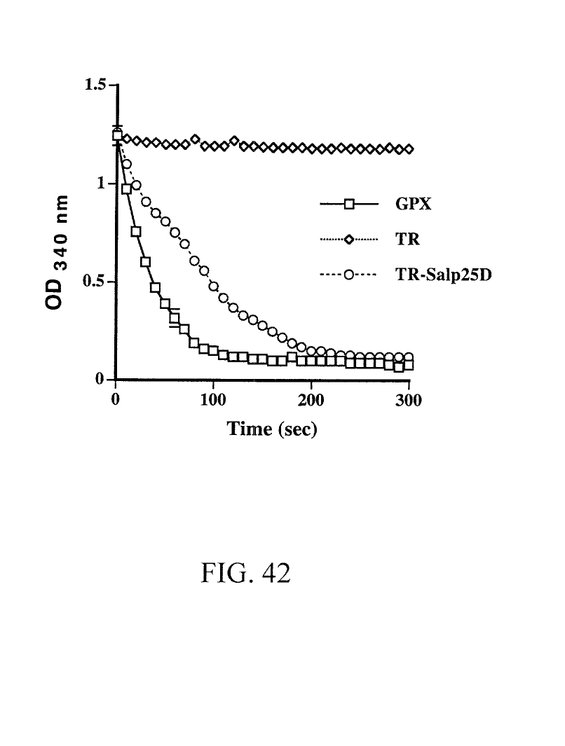

We prepared reaction mixtures (2.5 ml) containing 0.5 μmol of glutathione reductase (Sigma Chemical, St. Louis, Mo.), 0.1 μmol of NADPH, 5 μmol H₂O₂, 4.5 μmol NaN₃, 2 μmol glutathione and 50 μmol Tris-HCl, pH 8.0 for all assays. We then added fifteen, thirty and forty-five μg (330, 660 and 1,000 nmol/ml respectively) of the purified Salp25C-thioredoxin fusion protein, and equimolar concentrations of thioredoxin as a negative control, to the reaction mixture. We then incubated this mixture at 25° C. and monitored for 5 min in a spectrophotometer at 340 nm for a decrease in NADPH concentration. We compared the data from these experiments to the activity of a positive control, 100 nmol/ml bovine glutathione peroxidase (Sigma), and a negative control that contained no added protein (FIG. 42). We observed that 200 nm/ml bovine glutathione peroxidase cleaves the substrate (H₂O₂) approximately twice as fast (100 sec) as 1,000 nmol/ml recombinant Salp25C (200 sec). These experiments confirmed that Salp25C exhibits glutathione peroxidase activity.

### Example XVIII

### Salp14A and Salp9A Have Anticoagulant Activity

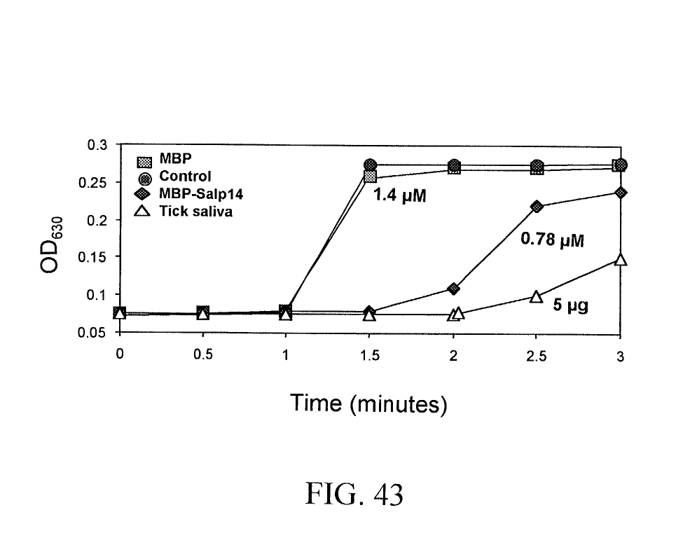

We monitored the anticoagulant activity of tick salivary proteins by adding either 20 μl of diluted tick saliva or 5 μg of purified Salp14A or Salp9A in phosphate buffer saline to 50 μl of normal human plasma in individual wells of a 96-well microtiter plate. We then added 20 μl of APTT-FS reagent (Sigma) to each well. We incubated the plates at 37° C. for 15 min and added 20 μl of calcium chloride (50 μM) to each of the wells and measured the time to thrombus formation over 3 min at 630 nm using a kinetic microplate reader. The clotting time was defined as the time (sec) following the addition of CaCl₂ at which the rate of increase in OD₆₃₀ (mOD/min) reached its maximum value. We determined this value using a computer software program and plotted the results as percent inhibition. Using this assay, we observed that 5 μg of total salivary protein delayed the clotting time of human plasma almost 2.5-fold. We then tested increasing concentrations of purified recombinant Salp14A or Salp9A (10-500 pM)in this assay. We observed that Salp14A and Salp9A clearly exhibit anticoagulation activity (FIG. 43). As a control is these assays, we used equimolar concentrations of the fusion partner maltose binding protein (MBP).

We then measured the ability of I. scapularis saliva and of purified recombinant Salp14A or Salp 9A to inhibit the activity of the clotting factor Xa in a single-stage chromogenic assay. We diluted clotting factor Xa to 200 pM in 10 mM HEPES (pH 7.5) containing 0.1% bovine serum albumin and 150 mM NaCl (HBSA). We then incubated 5 μl of saliva (corresponding to approximately 5 μg of Salp14A or Salp9A) at room temperature for 15 min with 100 μl of factor Xa in individual wells of a 96-well microtiter plate. We then added 50 μl of 1 mM S-2765 and measured substrate hydrolysis at 405 nm over a period of 5 min using a Vₘₐₓ kinetic microplate reader. We calculated the results as percent inhibition of factor Xa activity using the formula: % inhibition=[1−(inhibited rate/uninhibited rate)]×100. To monitor the kinetics of Salp14A-dependent inhibition of factor Xa activity, we measured the rate of hydrolysis of the chromogenic substrate S-2765 in the presence of increasing molar concentrations of Salp14A. We observed that recombinant Salp14A and Salp9A inhibited factor Xa activity suggesting that the anticoagulant activity is mediated by factor Xa inhibition.

1. An isolated, recombinant or synthetic DNA molecule comprising a DNA sequence which encodes a polypeptide, wherein said polypeptide is selected from the group consisting of:
(a) the Salp25A polypeptide having the amino-acid sequence shown in FIG. 16 (SEQ ID NO: 16);
(b) the Salp25B polypeptide having the amino-acid sequence shown in FIG. 18 (SEQ ID NO: 18);
(c) the Salp25C polypeptide having the amino-acid sequence shown in FIG. 2 (SEQ ID NO: 2);
(d) the Salp25D polypeptide having the amino-acid sequence shown in FIG. 4 (SEQ ID NO: 4);
(e) the Salp14A polypeptide having the amino-acid sequence shown in FIG. 6 (SEQ ID NO: 6);
(f) the Salp14B polypeptide having the amino-acid sequence shown in FIG. 24 (SEQ ID NO: 24);
(g) the Salp15 polypeptide having the amino-acid sequence shown in FIG. 8 (SEQ ID NO: 8);
(h) the Salp16A polypeptide having the amino-acid sequence shown in FIG. 10 (SEQ ID NO: 10);
(i) the Salp17 polypeptide having the amino-acid sequence shown in FIG. 12 (SEQ ID NO: 12);
(j) the Salp20 polypeptide having the amino-acid sequence shown in FIG. 14 (SEQ ID NO: 14);
(k) the Salp26A polypeptide having the amino-acid sequence shown in FIG. 20 (SEQ ID NO: 20);
(l) the Salp26B polypeptide having the amino-acid sequence shown in FIG. 22 (SEQ ID NO: 22);
(m) the Salp9 polypeptide having the amino-acid sequence shown in FIG. 26 (SEQ ID NO: 26);
(n) the Salp10 polypeptide having the amino-acid sequence shown in FIG. 28 (SEQ ID NO: 28);
(o) the salp13 polypeptide having the amino-acid sequence shown in FIG. 30 (SEQ ID NO: 30);
(p) the Salp9A polypeptide having the amino-acid sequence shown in SEQ ID NO: 36;
(q) fragments comprising at least 8 amino acids taken as a block from a polypeptide of (a)-(o); and
(r) a derivative of any one of the polypeptides of (a)-(q), said derivative being at least 75% identical in amino-acid sequence to the corresponding polypeptide of (a)-(q).

2. The isolated, recombinant or synthetic DNA molecule comprising the DNA sequence selected from the group consisting of:
(a) the DNA sequence set forth in FIGS. 1A-1B (SEQ ID NO: 1);
(b) the DNA sequence set forth in FIGS. 3A-3B (SEQ ID NO: 3);
(c) the DNA sequence set forth in FIG. 5 (SEQ ID NO: 5);
(d) the DNA sequence set forth in FIG. 7 (SEQ ID NO: 7);
(e) the DNA sequence set forth in FIG. 9 (SEQ ID NO: 9);
(f) the DNA sequence set forth in FIG. 11 (SEQ ID NO: 11);
(g) the DNA sequence set forth in FIG. 13 (SEQ ID NO: 13);
(h) the DNA sequence set forth in FIG. 15A-15B (SEQ ID NO: 15);
(i) the DNA sequence set forth in FIGS. 17A-17B (SEQ ID NO: 17);
(j) the DNA sequence set forth in FIGS. 19A-19B (SEQ ID NO: 19);
(k) the DNA sequence set forth in FIGS. 21A-21B (SEQ ID NO: 21);
(l) the DNA sequence set forth in FIG. 23 (SEQ ID NO: 23);
(m) the DNA sequence set forth in FIG. 25 (SEQ ID NO: 25);
(n) the DNA sequence set forth in FIG. 27 (SEQ ID NO: 27);
(o) the DNA sequence set forth in FIG. 29 (SEQ ID NO: 29); and
(p) the DNA sequence set forth in SEQ ID: 35.

3. A DNA molecule comprising a DNA sequence encoding a fusion protein, wherein the fusion protein comprises a polypeptide encoded by a DNA molecule according to claim 1.

4. A DNA molecule comprising a DNA sequence encoding a multimeric protein, which multimeric protein comprises a polypeptide encoded by a DNA molecule according to claim 1.

5. An expression vector comprising a DNA molecule according to claim 1.

6. An expression vector comprising a DNA molecule according to claim 2.

7. An expression vector comprising a DNA molecule according to any one of claims 3 and 4.

8. A host cell transformed with a DNA molecule according to claim 1.

9. A host cell transformed with a DNA molecule according to claim 2.

10. A host cell transformed with a DNA molecule according to any one of claims 3 and 4.

11. A host cell transformed with a DNA molecule according to claim 5.

12. A host cell transformed with a DNA molecule according to claim 6.

13. A host cell transformed with a DNA molecule according to claim 7.

14. The host cell according to claim 8, wherein said host cell is selected from the group consisting of: strains of E. coli; Pseudomonas, Bacillus; Streptomyces; yeast, fungi; animal cells, including human cells in tissue culture; plant cells; and insect cells.

15. The host cell according to claim 9, wherein said host cell is selected from the group consisting of: strains of E. coli; Pseudomonas, Bacillus; Streptomyces; yeast, fungi; animal cells, including human cells in tissue culture; plant cells; and insect cells.

16. The host cell according to claim 10, wherein said host cell is selected from the group consisting of: strains of E. coli; Pseudomonas, Bacillus; Streptomyces; yeast, fungi; animal cells, including human cells in tissue culture; plant cells; and insect cells.

17. A polypeptide encoded by a DNA molecule according to claim 1.

18. A method for producing a polypeptide according to claim 17, comprising the step of culturing a host cell according to claim 8.

19. A method for producing a polypeptide according to claim 17, comprising the step of culturing a host cell according to claim 10.

20. A polypeptide selected from the group consisting of:
(a) the Salp25A polypeptide having the amino-acid sequence shown in FIG. 16 (SEQ ID NO: 16);
(b) the Salp25B polypeptide having the amino-acid sequence shown in FIG. 18 (SEQ ID NO: 18);
(c) the Salp25C polypeptide having the amino-acid sequence shown in FIG. 2 (SEQ ID NO: 2);
(d) the Salp25D polypeptide having the amino-acid sequence shown in FIG. 4 (SEQ ID NO: 4);
(e) the Salp14A polypeptide having the amino-acid sequence shown in FIG. 6 (SEQ ID NO: 6);
(f) the Salp14B polypeptide having the amino-acid sequence shown in FIG. 24 (SEQ ID NO: 24);
(g) the Salp15 polypeptide having the amino-acid sequence shown in FIG. 8 (SEQ ID NO: 8);
(h) the Salp16A polypeptide having the amino-acid sequence shown in FIG. 10 (SEQ ID NO: 10);
(i) the Salp17 polypeptide having the amino-acid sequence shown in FIG. 12 (SEQ ID NO: 12);
(j) the Salp20 polypeptide having the amino-acid sequence shown in FIG. 14 (SEQ ID NO: 14);
(k) the Salp26A polypeptide having the amino-acid sequence shown in FIG. 20 (SEQ ID NO: 20);
(l) the Salp26B polypeptide having the amino-acid sequence shown in FIG. 22 (SEQ ID NO: 22);
(m) the Salp9 polypeptide having the amino-acid sequence shown in FIG. 26 (SEQ ID NO: 26);
(n) the Salp10 polypeptide having the amino-acid sequence shown in FIG. 28 (SEQ ID NO: 28);
(o) the Salp13 polypeptide having the amino-acid sequence shown in FIG. 30 (SEQ ID NO: 30);
(p) the Salp9A polypeptide having the amino-acid sequence shown in SEQ ID NO: 36;
(q) fragments comprising at least 8 amino-acids taken as a block from a polypeptide of (a)-(p); and
(r) a derivative of any one of the polypeptides of (a)-(p), said derivative being at least 75% identical in amino-acid sequence to the corresponding polypeptide of (a)-(p).

21. A fusion protein comprising a polypeptide according to claim 20.

22. The fusion protein according to claim 21, wherein said fusion protein comprises two or more I. scapularis polypeptides.

23. A multimeric protein comprising a polypeptide according to claim 20.

24. A pharmaceutical composition comprising a pharmaceutically acceptable carrier and a polypeptide according to claim 17 or claim 20.

25. A pharmaceutical composition comprising a pharmaceutically acceptable carrier and a component selected from the group consisting of: a fusion protein according to claim 21 or 22; and a multimeric protein according to claim 23.

26. The pharmaceutical composition according to claim 24, wherein the component is cross-linked to an immunogenic carrier.

27. The pharmaceutical composition according to claim 25, wherein the component is cross-linked to an immunogenic carrier.

28. The pharmaceutical composition according to any one of claims 24, 26 or 27, further comprising at least one additional non-I. scapularis polypeptide.

29. The pharmaceutical composition according to claim 25, further comprising at least one additional non-I. scapularis polypeptide.

30. The pharmaceutical composition according to claim 28, wherein the non-I. scapularis polypeptide is a protective polypeptide from a tick-borne pathogen.

31. The pharmaceutical composition according to claim 29, wherein the non-I. scapularis polypeptide is a protective polypeptide from a tick-borne pathogen.

32. The pharmaceutical composition according to claim 30 or 31, wherein the tick-borne pathogen is selected from the group consisting of: Borrelia burgdorferi, aoHGE, Babesia microti and arboviruses.

33. The pharmaceutical composition according to claim 32, wherein the non-I. scapularis polypeptide is a B. burgdorferi polypeptide.

34. A method for conferring tick immunity, comprising the step of administering to a subject a polypeptide according to claim 20.

35. A method for conferring tick immunity, comprising the step of administering to a subject a pharmaceutical composition according to claim 25.

36. A method for conferring tick immunity, comprising the step of administering to a subject a pharmaceutical composition according to claim 28.

37. A method for conferring tick immunity, comprising the step of administering to a subject a pharmaceutical composition according to claim 32.

38. A method for preventing infection by a tick-borne pathogen or a tick-borne disease, wherein the method comprises the step of administering to a subject a polypeptide according to claim 20.

39. A diagnostic kit comprising a polypeptide according to claim 20.

40. A diagnostic kit comprising a component selected from the group consisting of: a fusion protein according to claim 21 or 22; and a multimeric protein according to claim 23, and also comprising a means for detecting binding of said component to an antibody.

41. An antibody, or antigen-binding portion thereof, that binds to a polypeptide according to claim 20.

42. The antibody according to claim 41 which is polyclonal.

43. The antibody according to claim 41 which is monoclonal.

44. A diagnostic kit comprising an antibody according to any one of claims 41-43.

45. A method for detecting tick immunity or tick bite comprising the step of contacting a body fluid of a subject with a polypeptide according to claim 20.

46. A method for detecting tick immunity or tick bite comprising the step of contacting a body fluid of a subject with a fusion protein according to claim 21 or 22; and a multimeric protein according to claim 23.

47. A pharmaceutical composition comprising an antibody according to anyone of claims 41-43.

48. A vaccine comprising a polyclonal antibody according to claim 42.

49. A vaccine comprising a monoclonal antibody according to claim 43.

50. A method for conferring tick immunity comprising administering to a subject an antibody according to any one of claims 41-43, or a vaccine according to claim 48 or 49.

51. A vaccine comprising one or more polypeptides according to claim 20.

52. A method for inhibiting coagulation factor Xa activity comprising administering to a subject a polypeptide selected from the group consisting of:
(a) Salp14A;
(b) Salp9A; and
(c) a fragment of (a) or (b) having Xa inhibiting activity.

53. A method for inhibiting histamine activity comprising the step of administering a Salp25D polypeptide or a histamine-binding fragment thereof to a subject.

54. A method for inhibiting or preventing an inflammatory response comprising the step of administering to a subject a polypeptide selected from the group consisting of:
(a) a Salp15 polypeptide;
(b) a Salp25C polypeptide;
(c) a Salp13 polypeptide; and
(d) a fragment of (a)-(c) having the same activity.


SEQUENCE LISTING (39 sequences)

SEQ ID NO: 1 - DNA, 666 bp, Ixodes scapularis
atgggtcccctgaacctcggcgatcctttccccaacttcacctgcgacacgaccgagggcaagatcgacttccacgaatggctcggcaactcgtggggcatcctgttctcgcaccccgccgactacaccccggtgtgcacaagtgagctcgccagggcagcacagctgcaccacgtctttcagaagaagggtgtcaagctcatcgctctctcctgtgacagtgtggagagccaccgtggttggatcaaggacatcaacgcctttggggagctgccggacgggcccttcccgtaccccatcatcgccgatgagaagcgcgacattgccgtcaagctgggaatgttggaccccgtggagaaggacaaggaagggctgcctctcacctgcagggcggtgttcatcattggtcccgacaagaaaatgaagctctccatgctgtatcccgccacgactggaaggaactttgacgaggtcctgcgtgccaccgattccctgctggtgacggagaccaggaaggtggcgacgcctgctggttggcagaagggcaccccgtgcatggtcctgccttcggtgaccgaggaagagattctcaagctgttcccgacaggcatcaagcagtacgaagttccgtctggcaagaactacctccgaacaaccatggactga

SEQ ID NO: 2 - protein, 221 aa, Ixodes scapularis
MGPLNLGDPFPNFTCDTTEGKIDFHEWLGNSWGILFSHPADYTPVCTSELARAAQLHHVFQKKGVKLIALSCDSVESHRGWIKDINAFGELPDGPFPYPIIADEKRDIAVKLGMLDPVEKDKEGLPLTCRAVFIIGPDKKMKLSMLYPATTGRNFDEVLRATDSLLVTETRKVATPAGWQKGTPCMVLPSVTEEEILKLFPTGIKQYEVPSGKNYLRTTMD

SEQ ID NO: 3 - DNA, 666 bp, Ixodes scapularis
atgaagcttgttttaagcttggccgtttttgtgtgtggagtgttttggggaaccagtggctccacaagtactactacacgtcgagtgggaacgtatggctctacaggaacgactactcgtccagggacacgcggtgccagaatgattgtgactacagctccccctgaagaagacccttcgaaatacaaagaacaaaatgccaccagagtggtcgaaatgaacgctacgcaatgggtcaagtggcgcacgtatgatgtgacagatttcagcggtaacccagtgcagtgtgaaaattttagggttatggaaaagagaactccaacaaactactcattccagtacagatataaaagtaaaaatagttgggagacaatcgacgaaaccctaatcttgaaggatataggtgagcacggtttcccgaacgtcatgaactttcagaggactcctattggcatagcaacggacaatctcgtgttgtattccaactacgtgaactgcaccgttcttcgaatcccattcacaaatcaaggagagaggcattgcgacctgtggatggccaatctgactctttcccaagaaacccccgacgactgcctgaataagttttttgaatattgcaacacgacgcaaatttaccgcgtgtactaccctagttgtacaaactag

SEQ ID NO: 4 - protein, 221 aa, Ixodes scapularis
MKLVLSLAVFVCGVFWGTSGSTSTTTRRVGTYGSTGTTTRPGTRGARMIVTTAPPEEDPSKYKEQNATRVVEMNATQWVKWRTYDVTDFSGNPVQCENFRVMEKRTPTNYSFQYRYKSKNSWETIDETLILKDIGEHGFPNVMNFQRTPIGIATDNLVLYSNYVNCTVLRIPFTNQGERHCDLWMANLTLSQETPDDCLNKFFEYCNTTQIYRVYYPSCTN

SEQ ID NO: 5 - DNA, 378 bp, Ixodes scapularis
atggggttgaccggaaccatgctggtgttggtatctctggccttcttcgggagcgctgcagcccacaattgccagaacggaacgagacccgcatcggagcaagatagagaaggctgcgactattactgctggaacgctgagaccaaatcatgggaccaatttttctttggaaacggagaagaatgcttttacaacagtggtgatcacggaacatgtcaaaacggagaatgtcatttgacaaataattcaggtgggcccaacgaaactgatgactatactcctgcgcccactgagaagccgaagcaaaaaaagaagaaaactaagaagactaagaaacctaagcgcaagtcaaagaaagatcaggagaaaaacttatga

SEQ ID NO: 6 - protein, 125 aa, Ixodes scapularis
MGLTGTMLVLVSLAFFGSAAAHNCQNGTRPASEQDREGCDYYCWNAETKSWDQFFFGNGERCFYNSGDHGTCQNGECHLTNNSGGPNETDDYTPAPTEKPKQKKKKTKKTKKPKRKSKKDQEKNL

SEQ ID NO: 7 - DNA, 408 bp, Ixodes scapularis
atggaatctttcgtcgcaatgaaggtggtgtgcatactatttttggttggtgttgtcgctgcgaatgaaagcggcccaactaaagcagacgcatcaaccgctgacaaagatacgaagaaaaacaatgtgcaacttcgattccctaattatatttctaaccaccaaaagcttgccttgaaacttctgaaaatttgcaaggatagcaaatcttctcacaattcccttagttcccgttcgtccgatgtgataaacgacaaatacgtggacttcaagaactgtacatttctttgcaaacatggaaatgatgttaacgtgacattgaatttgccagaagacacgccttgtggaccgaatggacagacatgcgctgaaaagaataaatgcgttggccacattcccggatgttag

SEQ ID NO: 8 - protein, 135 aa, Ixodes scapularis
MESFVAMKVVCILFLVGVVAANESGPTKADASTADKDTKKNNVQLRFPNYISNHQKLALKLLKICKDSKSSHNSLSSRSSDVINDKYVDFKNCTFLCKHGNDVNVTLNLPEDTPCGPNGQTCAEKNKCVGHIPGC

SEQ ID NO: 9 - DNA, 435 bp, Ixodes scapularis
atggtggaggacggtatgaagaacaccggtattttatcctttattatgagaggaacaagactgatcatggcaacgtttgtgctgtttgcaatttgtaaaccaattgtcgccggcgttgtaatcagaggtacggaaaaccttgcgccgaaatgtgaacaaaagattaagcatctttgtgagaaccctacacatggtgagctaacggaggtcactgtgacggctcgccagtgccaagccacatgcacttatcgacccgaccctactagagatacggtggaagttgatggtatacctattaagaaccgtcattatgaaacagtcaccctgccagataaaatgccgtgcggttttggggcgagatgcaacaagaaaggggagtgcagttgcaactcttgcaatgaaaatataaaccacaacgagccaaggcaaacttaa

SEQ ID NO: 10 - protein, 144 aa, Ixodes scapularis
MVEDGMKNTGILSFIMRGTRLIMATFVLFAICKPIVAGVVIRGTENLAPKCEQKIKHLCENPTHGELTEVTVTARQCQATCTYRPDPTRDTVEVDGIPIKNRHYETVTLPDKMPCGFGARCNKKGECSCNSCNENINHNEPRQT

SEQ ID NO: 11 - DNA, 453 bp, Ixodes scapularis
atgaagcttgctataagcttggccgtttttgtgtgtggagttctagtgggagcctatggcaaaatgactactaccacaccacgagggagattgcgtggcagtatgactacagttccccctgaatacgacctatcaaaacaagaagaacaaaatgcaaccagagtggtccaaatgaacgctacacaatgggtcaagtggcgcacgtataatgtgacagatcttgtcagcggttaccctctgcagtgcgaaaatttcaaggttatggaaaagagaacaccgacaaactactctttacaatacaattataggagcggatatagttgggagacaatcaacgaagacctaattttgggatttctcaatctacggccccttcccccaaatgacatgttttttgcgaggactcctataggggaccaacccacaatttggtgttgtattccgactacatga

SEQ ID NO: 12 - protein, 150 aa, Ixodes scapularis
MKLAISLAVFVCGVLVGAYGKMTTTTPRGRLRGSMTTVPPEYDLSKQEEQNATRVVQMNATQWVKWRTYNVTDLVSGYPLQCENFKVMEKRTPTNYSLQYNYRSGYSWETINEDLILGFLNLRPLPPNDMFFARTPIGDQPTIWCCIPTT

SEQ ID NO: 13 - DNA, 552 bp, Ixodes scapularis
atgaggactgcgctcacctgtgcgcttttggcgatttcgtttctaggaagcccgtgttcgtccagcgaagacggtctagagcaagattccaaagtggaaactactacacaaaatctctacgaacgtcattatagaaataattctggattgtgcggggcacagtataggaattcaagccatgcggaagccgtttacaactgcacgctcagtcttttgcccccaaaggtgaatgaaacctgggaaggaattaggcatcgaattaataaaagcatacctgagttcgtaaggttgatgtgcaactttagtgttgtgatgcctgaagacttctacttagtttatatggggtcaaatggaaactcatactctgaagaggacgaagagagcacagacactgacaaagacagtaaaacggggtcttctgctgcagttgaagttacagaaaagctaatattaaaagcagaggaaaactgcacggcgcatataactggttggaccactgaagccccgaccacgctggaacctacggagactcaattcgaggcaatttcctct

SEQ ID NO: 14 - protein, 183 aa, Ixodes scapularis
MRTALTCALLAISFLGSPCSSSEDGLEQDSKVETTTQNLYERHYRNNSGLCGAQYRNSSHAEAVYNCTLSLLPPKVNETWEGIRHRINKSIPEFVRLMCNFSVVMPEDFYLVYMGSNGNSYSEEDEESTDTDKDSKTGSSAAVEVTEKLILKAEENCTAHITGWTTEAPTTLEPTETQFEAIS

SEQ ID NO: 15 - DNA, 669 bp, Ixodes scapularis
atgaagcttgttttaagcttggccgttcttttgtgtggagttcttgtgagaacctatggtaaagcacctcaagggggaagctacggcaaaacaagtagtactacacctcgagggatattatatggcagtacgactacagttccccctgaagaagacccgtcaaaactacgagaacaaaatgcaaccagagtggtccaaatgaacgctacacaatgggtcaagtggcgaacgtataatgtgacagatcctttacgtggtaactacccgctgcagtgtgaaaatttcaaggttatggaaaagagaacaccgttcaactactccttacagtacaaatataggagcggatatagttgggtgacacgtaccgaaaccctagttttggggattctcggattacagcctctcccaccaaatgacatgttctttcaaaggactcctattggcataccaacaaacaattacgtgttgtattccaactacgtgaactgcaccattcttcgaatcccaatgccaggacaaggacagaaccattgtgacctgtggatggcgaatatgactgtttctcaagaaccacccaaaatctgcagcgacaaattttatgaatattgcaacactacacaaaaattcaccgtttactttccaaattgtactgaaataagtagtttttag

SEQ ID NO: 16 - protein, 222 aa, Ixodes scapularis
MKLVLSLAVLLCGVLVRTYGKAPQGGSYGKTSSTTPRGILYGSTTTVPPEEDPSKLREQNATRVVQMNATQWVKWRTYNVTDPLRGNYPLQCENFKVMEKRTPFNYSLQYKYRSGYSWVTRTETLVLGILGLQPLPPNDMFFQRTPIGIPTNNYVLYSNYVNCTILRIPMPGQGQNHCDLWMANMTVSQEPPKICSDKFYEYCNTTQKFTVYFPNCTEISSF

SEQ ID NO: 17 - DNA, 669 bp, Ixodes scapularis
atgaagcttgttttaagcttggccgttcttgtgtgtggagttcttgtgagaacctatggcaaaacacatcaagtgggaagctacggcaaaccaagtagtactacacctcgagggatattatatggcagtacgactacagttccccctgaagaagacccggcaaaactacgagaacaaaatgcaaccagagtggtccaaatgaacgctacacaatgggtcaagtggcgcacgtttaatgtgacagatcctttccgtggtaactacccgctgcagtgtgaaaacttcaaggttatggaaaagagaacaccgttcaactactccttacagtacaaatataggagcggatatagttgggtgacacttaccgaaaccctagttttggggattctcggagaacagcgtctcccaccaaatgacatgttctttcagaggactcctattggcataccaacaaacaattacgtgttgtattccaactacgtgaactgcaccattcttcgaatcccaatgccaggacaaggacggaaccattgtgacctgtggatggcgaatatgactgtttctcaagaaccacccaaaatctgcagcgacaaattttatgaatattgcaacactacacaaaaattcaccgtttactttccaaattgtactgaaataagtggcttttag

SEQ ID NO: 18 - protein, 222 aa, Ixodes scapularis
MKLVLSLAVLVCGVLVRTYGKTHQVGSYGKPSSTTPRGILYGSTTTVPPEEDPAKLREQNATRVVQMNATQWVKWRTFNVTDPFRGNYPLQCENFKVMEKRTPFNYSLQYKYRSGYSWVTLTETLVLGILGEQRLPPNDMFFQRTPIGIPTNNYVLYSNYVNCTILRIPMPGQGRNHCDLWMANMTVSQEPPKICSDKFYEYCNTTQKFTVYFPNCTEISGF

SEQ ID NO: 19 - DNA, 702 bp, Ixodes scapularis
  modified_base (1)..(702): “n” represents a, t, c, g, other or unknown
atgagggctgtcattggagcgcttatgtatatattatttgccttctgctttgcaagcaatgaagtgtttgagaaatatccgaaaaacataggttctgatgagaaacttataacggttgcctttgttctggccggatttgacaatgcggatgtgaatatgaaatctgatgttggtgtttggcttcaggattcttatgatgaggctgcagataagttaagcaaagaattacaagttcaacttaaatttgagattaccgatatccaacacgcgcctcctgcccttacggacgaaattgcgtaccggacggttggtgggcaaatgcacggccctacaattctggatgccgtgaaaaatacttataaaaaacacctcaaccccgatatcatatctgttattacgaaagacaaattctacgatgacaagttcagcaataaaataggtttttcagggttcaccacactctgcgaagatatggtgcctattcttttgacgttcaattcggatattgaggacgacgttnatgcgaccgctaccctgttgtccaagctaattgagagcagtttggacccagttaaatggaaagaatttggnccgagaaaagactacttcgatggatgtaacataagacccnaacctaaaggagatacttataatgaatattacgtttttcctnttgacaaagctcctttttacgatttttaa

SEQ ID NO: 20 - protein, 233 aa, Ixodes scapularis
  MOD_RES (1)..(233): “Xaa” represents other or unknown amino acid
MRAVIGALMYILFAFCFASNEVFEKYPKNIGSDEKLITVAFVLAGFDNADVNMKSDVGVWLQDSYDEAADKLSKELQVQLKFEITDIQHAPPALTDEIAYRTVGGQMHGPTILDAVKNTYKKHLNPDIISVITKDKFYDDKFSNKIGFSGFTTLCEDMVPILLTFNSDIEDDVXATATLLSKLIESSLDPVKWKEFGPRKDYFDGCNIRPXPKGDTYNEYYVFPXDKAPFYDF

SEQ ID NO: 21 - DNA, 655 bp, Ixodes scapularis
atgaaagcatttttcagcctcgtgatagctgctctgtacccctctttgaaatgtactctaagctcttcaagctgcgaatgtcctgctccagaaacttttctgctggaagatcctaacttttttggattcagagatccatggcctttcctcagaagcccggagcgcttatacctgaagtatgcgccgatctgggaatatttggaaaaaataaagtgcactttcagcgacttcgtaagaaacgatagcagttacgagtttgtcaagcgaacactgtcctggataagcgtaggcgaacaacctagtagacacactatagaggtagagataacaggaacttccaaatcaaaatctgaagttaaagtgaccaaagggtacgaagagtatgatttcaatacggtgtacgccgacactagatgtctcatcctaagaatttcacggactcagaaggttccattaagatcatgtcttctttgggtgaaaaaaacatttctgaagaatccactgaggcactgtcgcttcttattcgacgtattctgcaactggaggagggaagactttaaaccagaaaaatactgtgacgaaggcgtggagaaaaaagatgaacgcccaactgtggcaggcaaccaaaattttggtactcctcggcctcccctga

SEQ ID NO: 22 - protein, 217 aa, Ixodes scapularis
MKAFFSLVIAALYPSLKCTLSSSSCECPAPETFLLEDPNFFGFRDPWPFLRSPERLYLKYAPIWEYLEKIKCTFSDFVRNDSSYEFVKRTLSWISVGEQPSRHTIEVEITGTSKSKSEVKVTKGYEEYDFNTVYADTRCLILRISRTQKVPLRSCLLWVKKTFLKNPLRHCRFLFDVFCNWRREDFKPEKYCDEGVEKKDERPTVAGNQNFGTPRPR

SEQ ID NO: 23 - DNA, 345 bp, Ixodes scapularis
  modified-base (274): a, t, c, g, other or unknown
atgcaactgacccttttcattgttattgttacctttacacatttgtcttgtgaggtacaatcggattccaacccgcttatctctggaaagatggaaaaattgccacaagattgtaaagatactctaatccaacaaatgcggaataaatgtggtgaaagtccattccagacccagcttgtagaagtcaaagactgctcatttgcatgtggagagtggcacaacaacggacagacaatgggaacaagtcgtcaaactactaatctgaaggatgggncctcttgcggataccgtaaaatatgcgtaggtggacactgtgtccagcaatgcctggtcgatttcgcttag

SEQ ID NO: 24 - protein, 114 aa, Ixodes scapularis
  MOD_RES (92): any, other or unknown amino acid
MQLTLFIVIVTFTHLSCEVQSDSNPLISGKMEKLPQDCKDTLIQQMRNKCGESPFQTQLVEVKDCSFACGEWHNNGQTMGTSRQTTNLKDGXSCGYRKICVGGHCVQQCLVDFA

SEQ ID NO: 25 - DNA, 246 bp, Ixodes scapularis
atgtgcaacttcactgttgctatgcctgataacttctacttactttatatgggggacgcaacgtcaaactacgactccgaagaggaggaacagagcacaggcactactgaagagtctcctgctgtgaaagttacagaacagcaaataacagacgcagagaacgcctgcacggcgaatataactggttggacccctccaaccacgccggaaccgacgaagtctcttgagcctgtggccgtcccctga

SEQ ID NO: 26 - protein, 81 aa, Ixodes scapularis
MCNFTVAMPDNFYLLYMGDATSNYDSEEEEQSTGTTEESPAVKVTEQQITDAENACTANITGWTPPTTPEPTKSLEPVAVP

SEQ ID NO: 27 - DNA, 291 bp, Ixodes scapularis
atgaaggcagccattgcagttctttgctttctcgttgcagtagcgtacgccattgtggtggaggcgaggatggcgaaccaaccgattgacgacggccccctcaatccaaaatgcgtaaaaccgaaaggttgccccggagatttcaagacggtttcctattacgacccgaataaaggatgtcaacttattaaactgggagaaaactgtaccgacaatggcaattaccccacacttgaggattgcaatcggcattgtctacctcctccagggaagcaaacgagactcagctga

SEQ ID NO: 28 - protein, 96 aa, Ixodes scapularis
MKAAIAVLCFLVAVAYAIVVEARMANQPIDDGPLNPKCVKPKGCPGDFKTVSYYDPNKGCQLIKLGENCTDNGNYPTLEDCNRHCLPPPGKQTRLS

SEQ ID NO: 29 - DNA, 345 bp, Ixodes scapularis; identical to SEQ ID NO: 23
  modified_base (274): a, t, c, g, other or unknown

SEQ ID NO: 30 - protein, 114 aa, Ixodes scapularis; identical to SEQ ID NO: 24
  modified-base (92): “Xaa” represents other or unknown

SEQ ID NO: 31 - protein, 8 aa, Ixodes scapularis
FFFENGEK

SEQ ID NO: 32 - DNA, 29 bp, Artificial Sequence
  Description of Artificial Sequence Oligonucleotide
gcccaaagaggaaaatttgtttcatacag

SEQ ID NO: 33 - DNA, 20 bp, Artificial Sequence
  Description of Artificial Sequence Oligonucleotide
gtcgacgtgagtcagcgcgc

SEQ ID NO: 34 - DNA, 21 bp, Artificial Sequence
  Description of Artificial Sequence Oligonucleotide
gatcagaggggactttccgag

SEQ ID NO: 35 - DNA, 225 bp, Ixodes scapularis
agaccggccactgaggagaagagagaaggctgcgactattactgctggaacaccgagaccaaatcatgggacaaatttttcttcgggaacggagaacgatgcttttacaacaatggtgatgagggattatgtcaaaacggagagtgccatttgacaacagattcaggtatgcccaatgacactgatgcaaaaatagaagaaaccgaagaagagttagaagcctaa

SEQ ID NO: 36 - protein, 74 aa, Ixodes scapularis
RPATEEKREGCDYYCWNTETKSWDKFFFGNGERCFYNNGDEGLCQNGECHLTTDSGMPNDTDAKIEETEEELEA

SEQ ID NO: 37 - protein, 224 aa, Bovine sp.
MPGGLLLGDEAPNFEANTTIGRIRFHDYLGDSWGILFSHPRDFTPVCTTELGRAAKLAPEFAKRNVKMIALSIDSVEDHLAWSKDINAYNGEEPTEKLPFPIIDDKNRDLAIQLGMLDPAEKDEKGMPVTARVVFIFGPDKKLKLSILYPATTGRNFDEILRVIISLQLTAEKRVATPVDWKNGDSVMVLPTIPEEEAKKLFPKGVFTKELPSGKKYLRYTPQP

SEQ ID NO: 38 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 39 - protein, 235 aa, Dirofilaria immitis
MTKGILLGDKFPDFRAETNEGFIPSFYDWIGKDSWAILFSHPRDFTPVCTTELARLVQLAPEFNKRNVKLIGLSCDSAESHRKWVDDIMAVCKMKCNDGDTCCSGNKLPFPIIADENRFLATELGMMDPDERDENGNALTARCVFIIGPEKTLKLSILYPATTGRNFDEILRVVDSLQLTAVKLVATPVDWKDGDDCVVLPTIDDTEAKKLFGEKINTIELPSGKHYLRMVAHPK